(19) United States
(12) Patent Application Publication
(10) Pub. No.: US 2026/0166021 A1
(43) Pub. Date: Jun. 18, 2026

(54) POLYMORPHIC FORMS OF A SOLUBLE EPOXIDE HYDROLASE INHIBITOR AND FORMULATIONS THEREOF

(71) Applicant: EicOsis, LLC, Davis, CA (US)

(72) Inventors: Sung Hee Hwang, Davis, CA (US); Cynthia B. McReynolds, Davis, CA (US); Bruce D. Hammock, Davis, CA (US)

(21) Appl. No.: 19/124,116

(22) PCT Filed: Oct. 25, 2023
(86) PCT No.: PCT/US2023/035918

§ 371 Date: Apr. 24, 2025

Related U.S. Application Data
(60) Provisional application No. 63/421,961, filed on Nov. 2, 2022.
(60) Provisional application No. 63/457,722, filed on Apr. 6, 2023.

(51) Int. Cl. A61K 31/444; C07D 471/04; C07D 487/04

(52) U.S. Cl. CPC A61K 31/444; C07D 471/04; C07D 487/04; A61K 2300/00

(57) ABSTRACT

In aspects, this invention covers crystalline forms of a soluble epoxide hydrolase inhibitor with ranges of properties advantageous for formulation and administration. In one aspect, the disclosure provides a method of treating a soluble epoxide hydrolase (sEH) mediated disorder or disease in a subject by administering to the subject the composition of the invention.

### CROSS-REFERENCE TO RELATED APPLICATIONS

This application claims benefit of priority under 35 U.S.C. § 119(e) of U.S. Provisional Application Nos. 63/421,961 filed Nov. 2, 2022, and 63/457,722 filed Apr. 6, 2023. The disclosures of the prior applications are considered part of and are herein incorporated by reference in the disclosure of this application in their entirety.

### STATEMENT OF GOVERNMENT RIGHTS

This disclosure was made in part from a grant from the National Institute of Neurological Disorders and Stroke (NINDS) Blueprint Neurotherapeutics Network UC3/UH3NS094258. The Government has certain rights to the invention.

### BACKGROUND

Soluble epoxide hydrolase (sEH, EC 3.3.2.10) is a bifunctional enzyme that in mammals is found in cytosol and cytosolic peroxisomal fractions. sEH performs dual functions, hydrolyzing biological epoxides and lipid phosphoryl groups, and thereby performs a central role in numerous lipid anabolic, catabolic, and signaling pathways. Accordingly, sEH dysregulation can exacerbate or manifest as metabolic, inflammatory, systemic, and cardiovascular disorders. Of particular clinical relevance, changes in sEH expression appear to contribute to numerous diseases, including certain cancers, neurodegenerative diseases, and forms of diabetes. Controlling sEH activity in diseased and risk-stage patients could thus provide a means for controlling pathogenesis in a range of diseases.

Nonetheless targeted sEH inhibition has remained a major challenge. sEH inhibitor design is simultaneously challenged by demands for aqueous solubility necessary for sEH delivery and extended hydrophobic structure important for sEH-binding affinity. The sEH active site, which is configured to bind a range of fatty acid substrates, contains two deep hydrophobic pockets flanking a hydrophilic epoxide-binding core. Stemming from these constraints, drug delivery is often limiting in sEH.

### SUMMARY

The present disclosure provides Formula (I) crystal forms with high purities, stabilities, and amenabilities for use with drug delivery platforms, and which therefore offer improved means for selectively targeting sEH. While Formula (I) is an effective sEH inhibitor, its amorphous form has limited solvent compatibilities, can have a relatively slow dissolution rate, and can be challenging to prepare in homogeneous, uniform fashion in pharmaceutical formulations. The properties of the crystal forms of Formula (I) disclosed herein differ from those of amorphous Formula (I), and thereby expand options for formulating Formula (I) beyond those which were previously available.

Aspects of the present disclosure provide a composition comprising an anhydrous crystalline form of Formula (I):

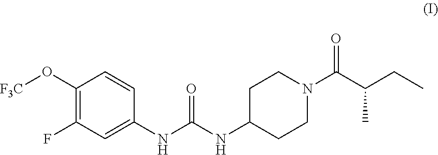 [SMILES: CC[C@H](C)C(=O)N1CCC(NC(=O)Nc2ccc(OC(F)(F)F)c(F)c2)CC1]

or a pharmaceutically acceptable salt thereof. In some embodiments, the anhydrous crystalline form of Formula (I) comprises less than 2.9% water content. In some embodiments, the anhydrous crystalline form of Formula (I) comprises less than 1.0% water content. In some embodiments, the anhydrous crystalline form of Formula (I) has a purity of at least 80%, at least 85%, at least 90%, at least 95%, at least 98%, or at least 99% by weight. In some embodiments, the composition comprises less than 5% Formula (I) degradation products by weight, less than 3% Formula (I) degradation products by weight, less than 2% Formula (I) degradation products by weight, or less than 1% Formula (I) degradation products by weight. In some embodiments, the anhydrous crystalline form of Formula (I) is characterized by an X-ray powder diffraction pattern substantially as set forth in any one of Panels A-L of FIG. 1.

Aspects of the present disclosure provide a composition comprising a crystalline form of Formula (I):

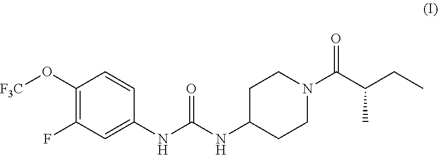 [SMILES: CC[C@H](C)C(=O)N1CCC(NC(=O)Nc2ccc(OC(F)(F)F)c(F)c2)CC1]

or a pharmaceutically acceptable salt thereof, wherein the crystalline form of Formula (I) comprises Form D as characterized by an X-ray powder diffraction substantially as set forth in Panel D of FIG. 1. In some embodiments, at least 80% of Formula (I) of the composition is Form D, at least 85% of Formula (I) of the composition is Form D, at least 90% of Formula (I) of the composition is Form D, at least 95% of Formula (I) of the composition is Form D, at least 98% of Formula (I) of the composition is Form D, or at least 99% of Formula (I) of the composition is Form D.

Aspects of the present disclosure provide a composition comprising a crystalline form of Formula (I):

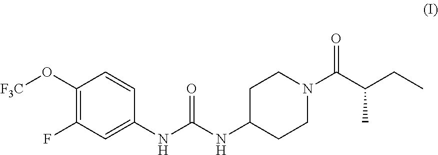 [SMILES: CC[C@H](C)C(=O)N1CCC(NC(=O)Nc2ccc(OC(F)(F)F)c(F)c2)CC1]

or a pharmaceutically acceptable salt thereof, wherein the crystalline form of Formula (I) comprises Form A as characterized by an X-ray powder diffraction pattern substantially as set forth in Panel A of FIG. 1. In some embodiments, at least 80% of Formula (I) of the composition is Form A, at least 85% of Formula (I) of the composition is Form A, at least 90% of Formula (I) of the composition is Form A, at least 95% of Formula (I) of the composition is Form A, at least 98% of Formula (I) of the composition is Form A, or at least 99% of Formula (I) of the composition is Form A.

Aspects of the present disclosure composition comprising a crystalline form of Formula (I):

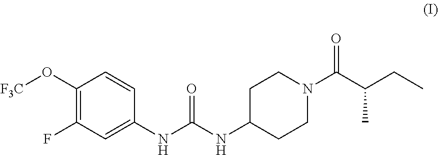 [SMILES: CC[C@H](C)C(=O)N1CCC(NC(=O)Nc2ccc(OC(F)(F)F)c(F)c2)CC1]

or a pharmaceutically acceptable salt thereof, wherein the crystalline form of Formula (I) comprises Form B as characterized by an X-ray powder diffraction pattern substantially as set forth in Panel B of FIG. 1. In some embodiments, at least 80% of Formula (I) of the composition is Form B, at least 85% of Formula (I) of the composition is Form B, at least 90% of Formula (I) of the composition is Form B, at least 95% of Formula (I) of the composition is Form B, at least 98% of Formula (I) of the composition is Form B, or at least 99% of Formula (I) of the composition is Form B.

Aspects of the present disclosure provide a composition comprising a crystalline form of Formula (I):

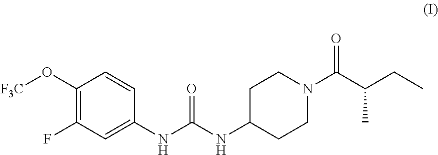 [SMILES: CC[C@H](C)C(=O)N1CCC(NC(=O)Nc2ccc(OC(F)(F)F)c(F)c2)CC1]

or a pharmaceutically acceptable salt thereof, wherein the crystalline form of Formula (I) comprises Form C as characterized by an X-ray powder diffraction pattern substantially as set forth in Panel C of FIG. 1. In some embodiments, at least 80% of Formula (I) of the composition is Form C, at least 85% of Formula (I) of the composition is Form C, at least 90% of Formula (I) of the composition is Form C, at least 95% of Formula (I) of the composition is Form C, at least 98% of Formula (I) of the composition is Form C, or at least 99% of Formula (I) of the composition is Form C.

Aspects of the present disclosure provide a composition comprising a crystalline form of Formula (I):

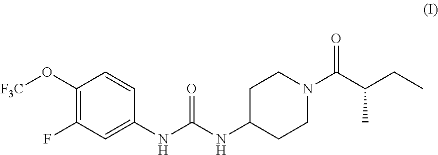 [SMILES: CC[C@H](C)C(=O)N1CCC(NC(=O)Nc2ccc(OC(F)(F)F)c(F)c2)CC1]

or a pharmaceutically acceptable salt thereof, wherein the crystalline form of Formula (I) comprises Form E as characterized by an X-ray powder diffraction pattern substantially as set forth in Panel E of FIG. 1. In some embodiments, at least 80% of Formula (I) of the composition is Form E, at least 85% of Formula (I) of the composition is Form E, at least 90% of Formula (I) of the composition is Form E, at least 95% of Formula (I) of the composition is Form E, at least 98% of Formula (I) of the composition is Form E, or at least 99% of Formula (I) of the composition is Form E.

Aspects of the present disclosure provide a composition comprising a crystalline form of Formula (I):

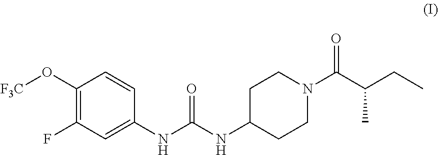 [SMILES: CC[C@H](C)C(=O)N1CCC(NC(=O)Nc2ccc(OC(F)(F)F)c(F)c2)CC1]

or a pharmaceutically acceptable salt thereof, wherein the crystalline form of Formula (I) comprises Form F as characterized by an X-ray powder diffraction pattern substantially as set forth in Panel F of FIG. 1. In some embodiments, at least 80% of Formula (I) of the composition is Form F, at least 85% of Formula (I) of the composition is Form F, at least 90% of Formula (I) of the composition is Form F, at least 95% of Formula (I) of the composition is Form F, at least 98% of Formula (I) of the composition is Form F, or at least 99% of Formula (I) of the composition is Form F.

Aspects of the present disclosure provide a composition comprising a crystalline form of Formula (I):

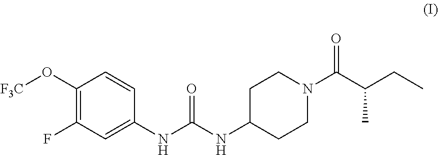 [SMILES: CC[C@H](C)C(=O)N1CCC(NC(=O)Nc2ccc(OC(F)(F)F)c(F)c2)CC1]

or a pharmaceutically acceptable salt thereof, wherein the crystalline form of Formula (I) comprises Form G as characterized by an X-ray powder diffraction pattern substantially as set forth in Panel G of FIG. 1. In some embodiments, at least 80% of Formula (I) of the composition is Form G, at least 85% of Formula (I) of the composition is Form G, at least 90% of Formula (I) of the composition is Form G, at least 95% of Formula (I) of the composition is Form G, at least 98% of Formula (I) of the composition is Form G, or at least 99% of Formula (I) of the composition is Form G.

A composition comprising a crystalline form of Formula (I):

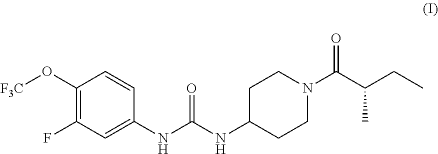 [SMILES: CC[C@H](C)C(=O)N1CCC(NC(=O)Nc2ccc(OC(F)(F)F)c(F)c2)CC1]

or a pharmaceutically acceptable salt thereof, wherein the crystalline form of Formula (I) comprises Form H as characterized by an X-ray powder diffraction pattern substantially as set forth in Panel H of FIG. 1. In some embodiments, at least 80% of Formula (I) of the composition is Form H, at least 85% of Formula (I) of the composition is Form H, at least 90% of Formula (I) of the composition is Form H, at least 95% of Formula (I) of the composition is Form H, at least 98% of Formula (I) of the composition is Form H, or at least 99% of Formula (I) of the composition is Form H.

Aspects of the present disclosure provide a composition comprising a crystalline form of Formula (I):

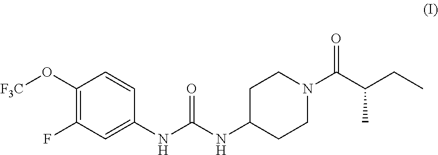 [SMILES: CC[C@H](C)C(=O)N1CCC(NC(=O)Nc2ccc(OC(F)(F)F)c(F)c2)CC1]

or a pharmaceutically acceptable salt thereof, wherein the crystalline form of Formula (I) comprises Form I as characterized by an X-ray powder diffraction pattern substantially as set forth in Panel I of FIG. 1. In some embodiments, at least 80% of Formula (I) of the composition is Form I, at least 85% of Formula (I) of the composition is Form I, at least 90% of Formula (I) of the composition is Form I, at least 95% of Formula (I) of the composition is Form I, at least 98% of Formula (I) of the composition is Form I, or at least 99% of Formula (I) of the composition is Form I.

Aspects of the present disclosure provide composition comprising a crystalline form of Formula (I):

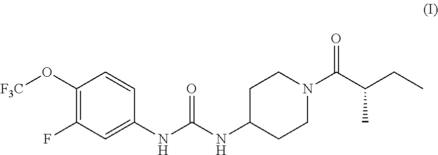 [SMILES: CC[C@H](C)C(=O)N1CCC(NC(=O)Nc2ccc(OC(F)(F)F)c(F)c2)CC1]

or a pharmaceutically acceptable salt thereof, wherein the crystalline form of Formula (I) comprises Form J as characterized by an X-ray powder diffraction pattern substantially as set forth in Panel J of FIG. 1. In some embodiments, at least 80% of Formula (I) of the composition is Form J, at least 85% of Formula (I) of the composition is Form J, at least 90% of Formula (I) of the composition is Form J, at least 95% of Formula (I) of the composition is Form J, at least 98% of Formula (I) of the composition is Form J, or at least 99% of Formula (I) of the composition is Form J.

Aspects of the present disclosure provide a composition comprising a crystalline form of Formula (I):

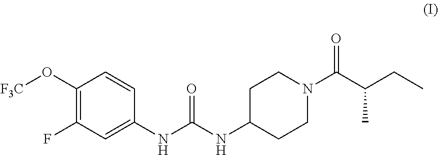 [SMILES: CC[C@H](C)C(=O)N1CCC(NC(=O)Nc2ccc(OC(F)(F)F)c(F)c2)CC1]

or a pharmaceutically acceptable salt thereof, wherein the crystalline form of Formula (I) comprises Form K as characterized by an X-ray powder diffraction pattern substantially as set forth in Panel K of FIG. 1. In some embodiments, at least 80% of Formula (I) of the composition is Form K, at least 85% of Formula (I) of the composition is Form K, at least 90% of Formula (I) of the composition is Form K, at least 95% of Formula (I) of the composition is Form K, at least 98% of Formula (I) of the composition is Form K, or at least 99% of Formula (I) of the composition is Form K.

Aspects of the present disclosure provide a composition comprising a crystalline form of Formula (I):

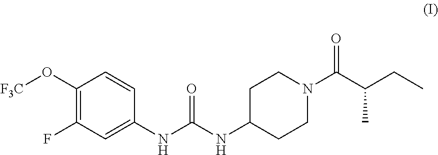 [SMILES: CC[C@H](C)C(=O)N1CCC(NC(=O)Nc2ccc(OC(F)(F)F)c(F)c2)CC1]

or a pharmaceutically acceptable salt thereof, wherein the crystalline form of Formula (I) comprises Form L as characterized by an X-ray powder diffraction pattern substantially as set forth in Panel L of FIG. 1. In some embodiments, at least 80% of Formula (I) of the composition is Form L, at least 85% of Formula (I) of the composition is Form L, at least 90% of Formula (I) of the composition is Form L, at least 95% of Formula (I) of the composition is Form L, at least 98% of Formula (I) of the composition is Form L, or at least 99% of Formula (I) of the composition is Form L.

In some embodiments, the crystalline form of Formula (I) comprises at least 98% Formula (I) by weight. In some embodiments, the crystalline form of Formula (I) comprises at least 99% Formula (I) by weight. In some embodiments, the crystalline form of Formula (I) has a mean particle size of between about 10 and about 100 microns. In some embodiments, the crystalline form of Formula (I) has a mean particle size of between about 2 and about 12 microns. In some embodiments, the crystalline form of Formula (I) has a melting temperature of between 140° C. and 145° C. In some embodiments, the crystalline form of Formula (I), wherein the crystalline form of Formula (I) has a melting temperature of between 145° C. and 150° C. In some embodiments, the crystalline form of Formula (I) has a heat of fusion of at least 25 J/g. In some embodiments, the crystalline form of Formula (I) has a heat of fusion of at least 50 J/g. In some embodiments, the crystalline form of Formula (I) has a heat of fusion of at least 55 J/g. In some embodiments, the crystalline form of Formula (I) has less than 10% solvent by weight. In some embodiments, the crystalline form of Formula (I) has less than 5% solvent by weight. In some embodiments, the crystalline form of Formula (I) is stable for at least 28 days at 25° C. and 0% humidity. In some embodiments, the crystalline form of Formula (I) is stable for at least 180 days at 25° C. and 0% humidity.

Aspects of the present disclosure provide a method for generating a crystalline form of Formula (I), the method comprising: dissolving Formula (I) in a solvent system comprising acetone, acetonitrile, dichloromethane, dioxane, isopropyl alcohol, methylethylketone, methyl isobutyl ketone, methyl tert-butyl ether, n-butyl alcohol, tetrahydrofuran, 2-methyl tetrahydrofuran, toluene, or a combination thereof at a temperature of between 35° C. and 80° C. in a solvent system, and cooling the solvent system to a temperature of between 0° C. and 30° C. In some embodiments, the dissolving is performed at a temperature between 50° C. and 75° C. In some embodiments, the cooling brings the solvent to a temperature of between 27° C. and −20° C. In some embodiments, the cooling brings the solvent system to a temperature of between 10° C. and −20° C. In some embodiments, the cooling is at a rate of between 0.1° C./hour and 600° C./hour. In some embodiments, the solvent system comprises a secondary solvent in which Formula (I) has a solubility of at most 5 mg/mL. In some embodiments, the secondary solvent is water or a C₅-C₁₂ alkane. In some embodiments, the secondary solvent is a hexane or a heptane. In some embodiments, the secondary solvent is c-hexane or n-heptane. In some embodiments, the method further comprises seeding solid Formula (I) into the solvent system following the dissolving. In some embodiments, the solid Formula (I) is in any one of Forms A-L. In some embodiments, the method further comprises adding an additional volume of the secondary solvent during or subsequent to the cooling.

Aspects of the present disclosure provide a method for generating a crystalline form of Formula (I), comprising: (i) dissolving Formula (I) in a solvent system comprising at least 90% methanol or at least 90% toluene, and cooling the solvent system to a temperature of between −20° C. and 30° C. at a rate of between 60° C./hour and 600° C./hour; (ii) dissolving Formula (I) in a solvent system comprising water and a solvent selected from the group consisting of acetonitrile and acetone, and cooling the solvent system to a temperature of between 0° C. and 30° C. at a rate of between 0.1° C./hour and 40° C./hour; (iii) dissolving Formula (I) to a concentration of at least about 0.4 mg/ml in a solvent system comprising water and acetonitrile, and cooling the solvent system to a temperature of between 0° C. and 30° C. at a rate of between 60° C./hour and 600° C./hour; (iv) incubating Form C of Formula (I) at a temperature of between 40° C. and 90° C. for at least 1 hour; or (v) a combination thereof. In some embodiments, (i) and (iii) comprise cooling the solvent system to a temperature of between −20° C. and 30° C. at a rate of between 60° C./hour and 150° C./hour. In some embodiments, (ii) comprises cooling the solvent system to a temperature of between 0° C. and 30° C. at a rate of between 1° C./hour and 30° C./hour. In some embodiments, the solvent system of (ii) comprises a ratio of water to the solvent selected from the group consisting of acetonitrile and acetone of between 5:1 and 1:5. In some embodiments, the solvent system of (ii) comprises a ratio of water to the solvent selected from the group consisting of acetonitrile and acetone of between 2:1 and 1:2. In some embodiments, the crystalline form is at least 80% Form A, at least 85% Form A, at least 90% Form A, at least 95% Form A, at least 98% Form A, or at least 99% Form A.

Aspects of the present disclosure provide a method for generating a crystalline form of Formula (I), comprising: (i) dissolving Formula (I) in a solvent system comprising at least 90% methanol, at least 90% ethanol, at least 90% isopropyl alcohol, or at least 90% n-butanol, and cooling the solvent system to a temperature of between −20° C. and 30° C. at a rate between 0.1° C./hour and 40° C./hour; (ii) dissolving Formula (I) in a solvent system comprising at least 90% ethanol, at least 90% isopropyl alcohol, or at least 90% n-butanol, and cooling the solvent system to a temperature of between −20° C. and 30° C. at a rate of between 60° C./hour and 600° C./hour; (iii) dissolving Formula (I) in a solvent system comprising water and n-propanol, and cooling the solvent system to a temperature of between 0° C. and 30° C. at a rate of between 60° C./hour and 600° C./hour; (iv) dissolving Formula (I) in a solvent system comprising a hexane and dioxane and cooling to a temperature of between 12° C. and 30° C. at a rate between 0.1° C./hour and 40° C./hour; (v) dissolving Formula (I) to a concentration of at most 0.1 mg/ml in a solvent system comprising a hexane and acetonitrile and cooling to a temperature of between −20° C. and 30° C. at a rate between 0.1° C./hour and 40° C./hour; or a combination thereof.

In some embodiments, (i) and (v) comprise cooling the solvent system to a temperature of between 0° C. and 30° C. at a rate of between 1° C./hour and 30° C./hour. In some embodiments, (ii) and (iii) comprise cooling the solvent system to a temperature of between 4° C. and 30° C. at a rate of between 60° C./hour and 150° C./hour. In some embodiments, the solvent system of (iii) comprises a volume-to-volume ratio of water to n-propanol of between 5:1 and 1:5. In some embodiments, the solvent system of (iv) comprises a volume-to-volume ratio of hexane to dioxane of between 5:1 and 1:5. In some embodiments, the solvent system of (v) comprises a volume-to-volume ratio of hexane to acetonitrile of between 10:1 and 150:1. In some embodiments, the crystalline form is at least 80% Form B, at least 85% Form B, at least 90% Form B, at least 95% Form B, at least 98% Form B, or at least 99% Form B.

Aspects of the present disclosure provide a method for generating a crystalline form of Formula (I), comprising: (i) dissolving Formula (I) in a solvent system comprising water and methanol, and cooling the solvent system to a temperature of between 0° C. and 30° C. at a rate of between 60° C./hour and 600° C./hour; (ii) incubating Formula (I) in water for at least one hour; (iii) incubating Formula (I) in a polyethylene glycol (PEG) water mixture comprising at least 50% water by volume for at least one hour; or (iv) a combination thereof. In some embodiments, (i) comprises cooling the solvent system to a temperature of between 0° C. and 15° C. at a rate of between 60° C./hour and 150° C./hour. In some embodiments, (ii) comprises incubating Formula (I) in water for at least one day. In some embodiments, (iii) comprises incubating Formula (I) in a polyethylene glycol (PEG) water mixture comprising at least 50% water by volume for at least one day. In some embodiments, (ii) and (iii) comprise incubating solid Formula (I). In some embodiments, the crystalline form is at least 80% Form C, at least 85% Form C, at least 90% Form C, at least 95% Form C, at least 98% Form C, or at least 99% Form C.

Aspects of the present disclosure provide a method for generating a crystalline form of Formula (I), comprising: (i) dissolving Formula (I) in a solvent system comprising a heptane and a solvent selected from the group consisting of dimethyl sulfoxide and n-methyl-2-pyrrolidone, and cooling the Formula (I) to a temperature of between −20° C. and 30° C. at a rate of between 0.1° C./hour and 600° C./hour; (ii) dissolving Formula (I) to a concentration of at most 0.25 mg/ml in a solvent system comprising water and dimethyl formamide, and cooling to a temperature of between 0° C. and 30° C. at a rate of between 0.1° C./hour and 40° C./hour; (iii) dissolving Formula (I) in a solvent system comprising a heptane and a solvent selected from the group consisting of ethanol, isopropyl alcohol, and ethyl acetate, and cooling to a temperature of between −20° C. and 30° C. at a rate of between 0.1° C./hour and 40° C./hour; (iv) dissolving Formula (I) in a solvent system comprising a hexane and dimethyl sulfoxide, and cooling to a temperature of between −20° C. and 30° C. at a rate of between 0.1° C./hour and 40° C./hour; (v) dissolving Formula (I) to a concentration of at least 0.4 mg/ml in a solvent system comprising a heptane and isopropyl alcohol, and cooling the solvent system to a temperature of between −20° C. and 30° C. at a rate of between 0.1° C./hour and 40° C./hour; (vi) incubating Formula (I) in a heptane for at least 1 day; (vii) incubating Formula (I) for at least 1 hour in a polyethylene glycol:water mixture comprising greater than 50% polyethylene glycol by volume; or (viii) a combination thereof. In some embodiments, (i) comprises cooling the solvent system to a temperature of between −10° C. and 15° C. at a rate of between 60° C./hour and 150° C./hour. In some embodiments, (ii)-(v) comprise cooling the solvent system to a temperature of between 10° C. and 30° C. at a rate of between 1° C./hour and 30° C./hour. In some embodiments, the solvent system of (i) comprises a ratio of heptane to the solvent selected from the group consisting of dimethyl sulfoxide and n-methyl-2-pyrrolidone of between 10:1 and 200:1. In some embodiments, the solvent system of (ii) comprises a ratio of water to dimethyl formamide of between 5:1 and 1:5. In some embodiments, the solvent system of (iii) comprises a ratio of heptane and the solvent selected from the group consisting of ethanol, isopropyl alcohol, and ethyl acetate of between 5:1 and 1:5. In some embodiments, the solvent system of (iv) comprises a ratio of hexane and dimethyl sulfoxide of between 10:1 and 200:1. In some embodiments, the solvent system of (v) comprises a ratio of heptane and isopropyl alcohol of between 3:1 and 40:1. In some embodiments, the crystalline form is at least 80% Form E, at least 85% Form D, at least 90% Form D, at least 95% Form D, at least 98% Form D, or at least 99% Form D.

Aspects of the present disclosure provide a method for generating a crystalline form of Formula (I), comprising: (i) dissolving Formula (I) in a solvent system comprising a heptane and a solvent selected from the group consisting of methanol, ethanol, acetonitrile, isopropyl alcohol, acetone, and n-propanol, and cooling the solvent system to a temperature of between −20° C. and 30° C. at a rate of between 60° C./hour and 600° C./hour; (ii) dissolving Formula (I) in a solvent system comprising a heptane and a solvent selected from the group consisting of methanol, ethanol, acetonitrile, and n-propanol, and cooling the solvent system to a temperature of between −20° C. and 30° C. at a rate of between 0.1° C./hour and 40° C./hour; (iii) dissolving Formula (I) in a solvent system comprising a heptane and methyl ethyl ketone, seeding the solvent system with Form E, and cooling the solvent system to a temperature of between −20° C. and 30° C. at a rate of between 0.1° C./hour and 40° C./hour; (iv) dissolving Formula (I) to a concentration of at most 0.25 mg/ml in a solvent system comprising a heptane and isopropyl alcohol, and cooling the solvent system to a temperature of between −20° C. and 30° C. at a rate of between 0.1° C./hour and 40° C./hour; (v) dissolving Formula (I) in a solvent system comprising a hexane and methanol, and cooling the solvent system to a temperature of between −20° C. and 30° C. at a rate of between 0.1° C./hour and 40° C./hour; (vi) incubating Formula (I) in a heptane for less than one day; or (vii) a combination thereof. In some embodiments, (i) comprises cooling the solvent system to a temperature of between −10° C. and 15° C. at a rate of between 60° C./hour and 150° C./hour. In some embodiments, (ii)-(v) comprise cooling the solvent system to a temperature of between 10° C. and 30° C. at a rate of between 1° C./hour and 30° C./hour. In some embodiments, the solvent system of (i) comprises a ratio of heptane to methanol, ethanol, acetonitrile, isopropyl alcohol, or n-propanol of between 5:1 and 200:1. In some embodiments, the solvent system of (i) comprises a ratio of heptane to acetone of between 1:1 and 15:1. In some embodiments, the solvent system of (ii) comprises a ratio of heptane to methanol, ethanol, acetonitrile, or n-propanol of between 5:1 and 200:1. In some embodiments, the solvent system of (iii) comprises a ratio of heptane and methyl ethyl ketone of between 1:1 and 15:1. In some embodiments, the solvent system of (iv) comprises a ratio of heptane and isopropyl alcohol of between 5:1 and 100:1. In some embodiments, the solvent system of (v) comprises a ratio of hexane and methanol of between 10:1 and 200:1. In some embodiments, (vi) comprises incubating solid Formula (I) in heptane for less than one day. In some embodiments, the crystalline form is at least 80% Form E, at least 85% Form E, at least 90% Form E, at least 95% Form E, at least 98% Form E, or at least 99% Form E.

Aspects of the present disclosure provide a method for generating a crystalline form of Formula (I), comprising: (i) dissolving Formula (I) in a solvent system comprising water and a solvent selected from the group consisting of methanol, ethanol, isopropyl alcohol, tetrahydrofuran, acetone, dimethyl sulfoxide, dimethyl formamide, N-Methyl and -2-pyrrolidone (NMP), and cooling the solvent system to a temperature of between 0° C. and 30° C. at a rate of between 60° C./hour and 600° C./hour; (ii) dissolving Formula (I) to a concentration of at most 0.25 mg/ml in a solvent system comprising water and acetonitrile, and cooling the solvent system to a temperature of between 0° C. and 30° C. at a rate of between 60° C./hour and 600° C./hour; (iii) dissolving Formula (I) in a solvent system comprising a heptane and dimethyl formamide and cooling the solvent system to a temperature of between −20° C. and 30° C. at a rate of between 60° C./hour and 600° C./hour; (iv) dissolving Formula (I) in a solvent system comprising a hexane and a solvent selected from the group consisting of ethanol, NMP, and n-propanol, and cooling the solvent system to a temperature of between −20° C. and 30° C. at a rate of between 60° C./hour and 600° C./hour; (v) dissolving Formula (I) in a solvent system comprising a hexane and dioxane, and cooling the solvent system to a temperature of between 4° C. and 30° C. at a rate of between 60° C./hour and 600° C./hour; (vi) dissolving Formula (I) in a solvent system comprising water and a solvent selected from the group consisting of ethanol, tetrahydrofuran, dimethylformamide, and n-propanol, and cooling the solvent system to a temperature of between 0° C. and 30° C. at a rate of between 0.1° C./hour and 40° C./hour; (vii) dissolving Formula (I) in a solvent system comprising a heptane and dimethyl sulfoxide, and cooling the solvent system to a temperature of between −20° C. and 30° C. at a rate of between 0.1° C./hour and 40° C./hour; (viii) dissolving Formula (I) in a solvent system comprising a hexane and a solvent selected from the group consisting of methanol, isopropyl alcohol, dimethyl formamide, and n-propanol, and cooling the solvent system to a temperature of between −20° C. and 30° C. at a rate of between 0.1° C./hour and 40° C./hour; or (ix) a combination thereof. In some embodiments, (i)-(iv) comprise cooling the solvent system to a temperature of between 0° C. and 15° C. at a rate of between 60° C./hour and 150° C./hour. In some embodiments, (v) comprises cooling the solvent system to a temperature of between 4° C. and 15° C. at a rate of between 60° C./hour and 150° C./hour. In some embodiments, (vi)-(viii) comprise cooling the solvent system to a temperature of between 10° C. and 30° C. at a rate of between 1° C./hour and 30° C./hour. In some embodiments, the solvent system of (i) comprises a ratio of water to methanol, ethanol, isopropyl alcohol, tetrahydrofuran, acetone, dimethyl sulfoxide, dimethyl formamide, or NMP of between 5:1 and 1:5. In some embodiments, the solvent system of (ii) comprises a ratio of water to acetonitrile of between 5:1 and 1:5. In some embodiments, the solvent system of (iii) comprises a ratio of heptane to dimethyl formamide of between 10:1 and 200:1. In some embodiments, the solvent system of (iv) comprises a ratio of hexane to ethanol, NMP, or n-propanol of between 5:1 and 100:1. In some embodiments, the solvent system of (v) comprises a ratio of hexane and dioxane of between 5:1 and 1:5. In some embodiments, the solvent system of (vi) comprises a ratio of water to ethanol, tetrahydrofuran, dimethylformamide, or n-propanol of 5:1 and 1:5. In some embodiments, the solvent system of (vii) comprises a ratio of heptane to dimethyl sulfoxide of between 10:1 and 200:1. In some embodiments, the solvent system of (viii) comprises hexane to methanol, isopropyl alcohol, dimethyl formamide, or n-propanol of between 10:1 and 200:1. In some embodiments, the crystalline form is at least 80% Form F, at least 85% Form F, at least 90% Form F, at least 95% Form F, at least 98% Form F, or at least 99% Form F.

Aspects of the present disclosure provide a method for generating a crystalline form of Formula (I), comprising: (i) dissolving Formula (I) in a solvent system comprising at least 90% tetrahydrofuran or at least 90% methyl ethyl ketone, and cooling the solvent system to a temperature of between −20° C. and 30° C. at a rate of between 0.1° C./hour and 40° C./hour; (ii) dissolving Formula (I) in a solvent system comprising at least 90% dioxane, and cooling the solvent system to a temperature of between 12° C. and 30° C. at a rate of between 0.1° C./hour and 600° C./hour; (iii) dissolving Formula (I) in a solvent system comprising at least 90% tetrahydrofuran, and cooling the solvent system to a temperature of between −20° C. and 30° C. at a rate of between 60° C./hour and 600° C./hour; (iv) dissolving Formula (I) in a solvent system comprising water and dioxane, and cooling the solvent system to a temperature of between 12° C. and 30° C. at a rate of between 0.1° C./hour and 600° C./hour; (v) dissolving Formula (I) in a solvent system comprising a heptane and dioxane, and cooling the solvent system to a temperature of between 4° C. and 30° C. at a rate of between 0.1° C./hour and 600° C./hour; (vi) dissolving Formula (I) in a solvent system comprising a heptane and tetrahydrofuran, and cooling the solvent system to a temperature of between −20° C. and 30° C. at a rate of between 60° C./hour and 600° C./hour; (vii) dissolving Formula (I) to a concentration of at most 0.25 mg/ml in a solvent system comprising a hexane and acetone, and cooling the solvent system to a temperature of between −20° C. and 30° C. at a rate of between 60° C./hour and 600° C./hour; (viii) dissolving Formula (I) in a solvent system comprising a hexane and tetrahydrofuran, and cooling the solvent system to a temperature of between −20° C. and 30° C. at a rate of between 0.1° C./hour and 40° C./hour; (ix) dissolving Formula (I) to a concentration of at least 0.4 mg/ml in a solvent system comprising a hexane and dioxane, and cooling the solvent system to a temperature of between 12° C. and 30° C. at a rate of between 0.1° C./hour and 40° C./hour; or (x) a combination thereof. In some embodiments, (i) comprises cooling the solvent system to a temperature of between 0° C. and 30° C. at a rate of between 1° C./hour and 30° C./hour. In some embodiments, (iv) comprises a ratio of water to dioxane of between 5:1 and 1:5. In some embodiments, (v) comprises a ratio of heptane to dioxane of between 3:1 and 60:1. In some embodiments, (vi) comprises a ratio of heptane to tetrahydrofuran of 5:1 and 1:5. In some embodiments, (viii) comprises a ratio of hexane and tetrahydrofuran of between 5:1 and 1:5. In some embodiments, (ix) comprises a ratio of hexane to dioxane of 5:1 and 1:5. In some embodiments, the crystalline form is at least 80% Form G, at least 85% Form G, at least 90% Form G, at least 95% Form G, at least 98% Form G, or at least 99% Form G.

Aspects of the present disclosure provide a method for generating a crystalline form of Formula (I), comprising: (i) dissolving Formula (I) in a solvent system comprising at least 90% 2-methyl tetrahydrofuran or at least 90% isopropyl acetate, and cooling the solvent system to a temperature of between −20° C. and 30° C. at a rate of between 0.1° C./hour and 600° C./hour; (ii) dissolving Formula (I) in a solvent system comprising at least 90% methyl isobutyl ketone, and cooling the solvent system to a temperature of between −20° C. and 30° C. at a rate of between 0.1° C./hour and 40° C./hour; (iii) dissolving Formula (I) in a solvent system comprising a hexane and a solvent selected from the group consisting of acetonitrile and tetrahydrofuran, and cooling the solvent system to a temperature of between −20° C. and 30° C. at a rate of between 60° C./hour and 600° C./hour; (iv) dissolving Formula (I) to a concentration of at least 0.4 mg/ml in a solvent system comprising a hexane and acetone, and cooling the solvent system to a temperature of between −20° C. and 30° C. at a rate of between 60° C./hour and 600° C./hour; (v) dissolving Formula (I) in a solvent system comprising a hexane and acetone, and cooling the solvent system to a temperature of between −20° C. and 30° C. at a rate of between 0.1° C./hour and 40° C./hour; (vi) dissolving Formula (I) to a concentration of at least 0.5 mg/ml in a solvent system comprising a hexane and acetonitrile, and cooling the solvent system to a temperature of between −20° C. and 30° C. at a rate of between 0.1° C./hour and 40° C./hour; or (vii) a combination thereof. In some embodiments, (i), (ii), (v), and (vi) comprise cooling the solvent system to a temperature of between 0° C. and 30° C. at a rate of between 1° C./hour and 30° C./hour. In some embodiments, (i), (iii), and (iv) comprise cooling the solvent system to a temperature of between 0° C. and 30° C. at a rate of between 60° C./hour and 150° C./hour. In some embodiments, (iii) or (vi) comprises a ratio of hexane to acetonitrile of between 5:1 and 100:1. In some embodiments, (iii) comprises a ratio of hexane to tetrahydrofuran of between 5:1 and 1:5. In some embodiments, (iv) or (v) comprises ratios of hexane to acetone of between 5:1 and 1:5. In some embodiments, the crystalline form is at least 80% Form H, at least 85% Form H, at least 90% Form H, at least 95% Form H, at least 98% Form H, or at least 99% Form H.

Aspects of the present disclosure provide a method for generating a crystalline form of Formula (I), comprising: (i) dissolving Formula (I) in a solvent system comprising at least 90% dichloromethane, and cooling the solvent system to a temperature of between −20° C. and 30° C. at a rate of between 0.1° C./hour and 600° C./hour; (ii) dissolving Formula (I) to a concentration of at most 0.25 mg/ml in a solvent system comprising at least 90% acetonitrile, and cooling the solvent system to a temperature of between −20° C. and 30° C. at a rate of between 60° C./hour and 600° C./hour; or (iii) a combination thereof. In some embodiments, (i) and (ii) comprise cooling to a temperature of between 0° C. and 30° C. at a rate of between 60° C./hour and 150° C./hour. In some embodiments, the crystalline form is at least 80% Form I, at least 85% Form I, at least 90% Form I, at least 95% Form I, at least 98% Form I, or at least 99% Form I.

Aspects of the present disclosure provide a method for generating a crystalline form of Formula (I), the method comprising: dissolving Formula (I) in a solvent system comprising at least 90% toluene, and cooling the solvent system to a temperature of between −20° C. and 30° C. at a rate of between 0.1° C./hour and 40° C./hour. In some embodiments, the method comprises cooling the solvent system to a temperature of between 0° C. and 30° C. at a rate of between 1° C./hour and 30° C./hour. In some embodiments, the crystalline form is at least 80% Form J, at least 85% Form J, at least 90% Form J, at least 95% Form J, at least 98% Form J, or at least 99% Form J.

Aspects of the present disclosure provide a method for generating a crystalline form of Formula (I), comprising: (i) dissolving Formula (I) in a solvent system comprising at least 90% methyl tert-butyl ether, and cooling the solvent system to a temperature of between −20° C. and 30° C. at a rate of between 0.1° C./hour and 600° C./hour; (ii) incubating Formula (I) in methyl tert-butyl ether for at least one hour; or (iii) a combination thereof. In some embodiments, the method comprises cooling the solvent system to a temperature of between 0° C. and 30° C. at a rate of between 1° C./hour and 150° C./hour. In some embodiments, the crystalline form is at least 80% Form K, at least 85% Form K, at least 90% Form K, at least 95% Form K, at least 98% Form K, or at least 99% Form K.

Aspects of the present disclosure provide a method for generating a crystalline form of Formula (I), comprising: (i) dissolving Formula (I) to a concentration of at least 0.4 mg/ml in a solvent system comprising water and isopropyl alcohol, and cooling the solvent system to a temperature of between 0° C. and 30° C. at a rate of between 0.1° C./hour and 40° C./hour; (ii) dissolving Formula (I) to a concentration of at least about 0.2 mg/ml in a solvent system comprising a hexane and acetonitrile, and cooling the solvent system to a temperature of between −20° C. and 30° C. at a rate of between 0.1° C./hour and 40° C./hour; (iii) dissolving Formula (I) in a solvent system comprising a heptane and tetrahydrofuran, and cooling the solvent system to a temperature of between −20° C. and 30° C. at a rate of between 60° C./hour and 600° C./hour; or (iv) a combination thereof. In some embodiments, (i) and (ii) comprise cooling the solvent system to a temperature of between 10° C. and 30° C. at a rate of between 1° C./hour and 40° C./hour. In some embodiments, (iii) comprises cooling the solvent system to a temperature of between 0° C. and 30° C. at a rate of between 60° C./hour and 150° C./hour. In some embodiments, (i) comprises a ratio of water to isopropyl alcohol of between 5:1 and 1:5. In some embodiments, (ii) comprises a ratio of hexane to acetonitrile of between 5:1 and 1:5. In some embodiments, the crystalline form is at least 80% Form L, at least 85% Form L, at least 90% Form L, at least 95% Form L, at least 98% Form L, or at least 99% Form L.

Aspects of the present disclosure provide a method of treating a soluble epoxide hydrolase (sEH) mediated disorder or disease in a subject, comprising administering to the subject the composition, thereby treating the disorder or disease in the subject. In some embodiments, the she mediated disorder or disease is selected from the group consisting of pain, a seizure disorder, epilepsy, Parkinson's disease, Alzheimer's disease, depression, spinal cord injury, peripheral nerve injury, stroke, multiple sclerosis, cognitive disfunction, nephropathy, cardiomyopathy, wound healing and inflammation. In some embodiments, the sEH mediated disorder or disease is selected from the group consisting of pain, a seizure disorder, nephropathy, cardiomyopathy, wound healing and inflammation. In some embodiments, the pain is neuropathic pain. In some embodiments, the neuropathic pain is associated with a nerve injury. In some embodiments, the nerve injury results from diabetes or other disease. In some embodiments, the pain is diabetic neuropathic pain. In some embodiments, the pain is inflammatory pain. In some embodiments, the seizure disorder is epilepsy.

Aspects of the present disclosure provide a composition comprising a crystalline form of Formula (I):

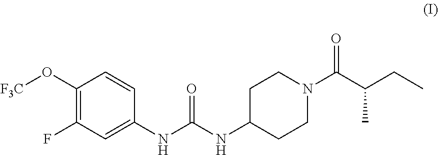 [SMILES: CC[C@H](C)C(=O)N1CCC(NC(=O)Nc2ccc(OC(F)(F)F)c(F)c2)CC1]

or a pharmaceutically acceptable salt thereof, wherein the crystalline form of Formula (I) comprises Form A as characterized by an X-ray powder diffraction pattern comprising peaks at 3.3±0.3°2θ, 30.3±0.3°2θ, and 20.0±0.3°2θ. In some embodiments, the X-ray powder diffraction pattern further comprises at least one, at least two, or at least three peaks selected from 12.1±0.3°2θ, 15.6±0.3°2θ, and 6.0±0.3°2θ. In some embodiments, the X-ray powder diffraction pattern further comprises at least one, at least two, or at least three peaks selected from 21.7±0.3°2θ, 10.6±0.3°2θ, and 21.6±0.3°2θ.

Aspects of the present disclosure provide a composition comprising a crystalline form of Formula (I):

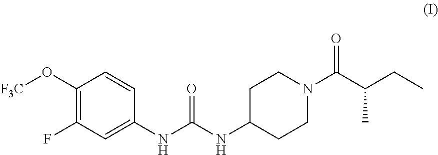 [SMILES: CC[C@H](C)C(=O)N1CCC(NC(=O)Nc2ccc(OC(F)(F)F)c(F)c2)CC1]

or a pharmaceutically acceptable salt thereof, wherein the crystalline form of Formula (I) comprises Form B as characterized by an X-ray powder diffraction pattern comprising peaks at 12.2±0.3°2θ, 3.5±0.3°2θ, and 17.2±0.3°2θ. In some embodiments, the X-ray powder diffraction pattern further comprises at least one, at least two, or at least three peaks selected from 19.6±0.3°2θ, 13.1±0.3°2θ, and 18.0±0.3°2θ. In some embodiments, the X-ray powder diffraction pattern further comprises at least one, at least two, at least three, or at least four peaks selected from 20.2±0.3°2θ, 14.1±0.3°2θ, 17.6±0.3°2θ, and 14.8±0.3°2θ.

Aspects of the present disclosure provide a composition comprising a crystalline form of Formula (I):

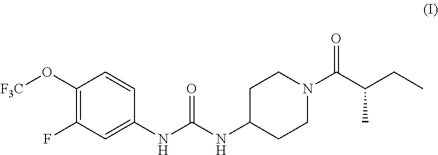 [SMILES: CC[C@H](C)C(=O)N1CCC(NC(=O)Nc2ccc(OC(F)(F)F)c(F)c2)CC1]

or a pharmaceutically acceptable salt thereof, wherein the crystalline form of Formula (I) comprises Form C as characterized by an X-ray powder diffraction pattern comprising peaks at 16.3±0.3°2θ, 16.1±0.3°2θ, and 3.2±0.3°2θ. In some embodiments, the X-ray powder diffraction pattern further comprises at least one, at least two, or at least three peaks selected from 21.6±0.3°2θ, 23.2±0.3°2θ, and 21.7±0.3°2θ. In some embodiments, the X-ray powder diffraction pattern further comprises at least one, at least two, or at least three peaks selected from 16.5±0.3°2θ, 21.4±0.3°2θ, and 10.7±0.3°2θ.

Aspects of the present disclosure provide a composition comprising a crystalline form of Formula (I):

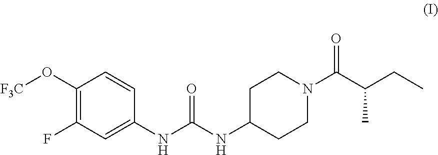 [SMILES: CC[C@H](C)C(=O)N1CCC(NC(=O)Nc2ccc(OC(F)(F)F)c(F)c2)CC1]

or a pharmaceutically acceptable salt thereof, wherein the crystalline form of Formula (I) comprises Form D as characterized by an X-ray powder diffraction pattern comprising peaks at 20.1±0.3°2θ, 18.3±0.3°2θ, and 18.1±0.3°2θ. In some embodiments, the X-ray powder diffraction pattern further comprises at least one or at least two peaks selected from 20.3±0.3°2θ and 17.1±0.3°2θ. In some embodiments, the X-ray powder diffraction pattern further comprises at least one, at least two, at least three, or at least four peaks selected from 3.4±0.3°2θ, 19.6±0.3°2θ, 23.4±0.3°2θ, and 25.1±0.3°2θ.

Aspects of the present disclosure provide a composition comprising a crystalline form of Formula (I):

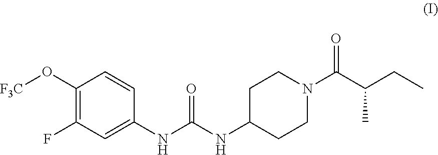 [SMILES: CC[C@H](C)C(=O)N1CCC(NC(=O)Nc2ccc(OC(F)(F)F)c(F)c2)CC1]

or a pharmaceutically acceptable salt thereof, wherein the crystalline form of Formula (I) comprises Form E as characterized by an X-ray powder diffraction pattern comprising peaks at 13.4±0.3°2θ, 11.2±0.3°2θ, and 3.1±0.3°2θ. In some embodiments, the X-ray powder diffraction pattern further comprises at least one, at least two, or at least three peaks selected from 9.0±0.3°2θ, 22.2±0.3°2θ, and 14.3±0.3°2θ. In some embodiments, the X-ray powder diffraction pattern further comprises at least one, at least two, or at least three peaks selected from 14.9±0.3°2θ, 18.4±0.3°2θ, and 16.8±0.3°2θ.

Aspects of the present disclosure provide a composition comprising a crystalline form of Formula (I):

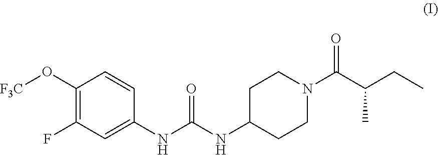 [SMILES: CC[C@H](C)C(=O)N1CCC(NC(=O)Nc2ccc(OC(F)(F)F)c(F)c2)CC1]

or a pharmaceutically acceptable salt thereof, wherein the crystalline form of Formula (I) comprises Form F as characterized by an X-ray powder diffraction pattern comprising peaks at 14.6±0.3°2θ, 3.4±0.3°2θ, and 9.7±0.3°2θ. In some embodiments, the X-ray powder diffraction pattern further comprises at least one, at least two, or at least three peaks selected from 18.1±0.3°2θ, 20.2±0.3°2θ, and 16.7±0.3°2θ. In some embodiments, the X-ray powder diffraction pattern further comprises at least one, at least two, or at least three peaks selected from 17.6±0.3°2θ, 19.2±0.3°2θ, and 17.3±0.3°2θ.

Aspects of the present disclosure provide a composition comprising a crystalline form of Formula (I):

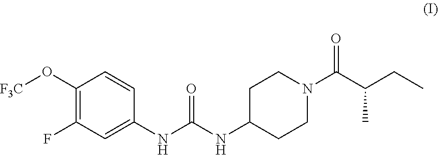 [SMILES: CC[C@H](C)C(=O)N1CCC(NC(=O)Nc2ccc(OC(F)(F)F)c(F)c2)CC1]

or a pharmaceutically acceptable salt thereof, wherein the crystalline form of Formula (I) comprises Form G as characterized by an X-ray powder diffraction pattern comprising peaks at 18.2±0.3°2θ, 3.2±0.3°2θ, and 18.0±0.3°2θ. In some embodiments, the X-ray powder diffraction pattern further comprises at least one, at least two, or at least three peaks selected from 10.8±0.3°2θ, 19.2±0.3°2θ, and 5.4±0.3°2θ. In some embodiments, the X-ray powder diffraction pattern further comprises at least one or at least two peaks selected from 10.6±0.3°2θ and 21.7±0.3°2θ.

Aspects of the present disclosure provide a composition comprising a crystalline form of Formula (I):

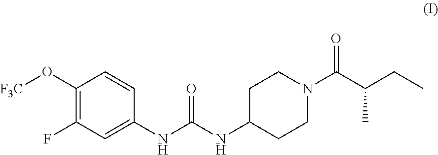 [SMILES: CC[C@H](C)C(=O)N1CCC(NC(=O)Nc2ccc(OC(F)(F)F)c(F)c2)CC1]

or a pharmaceutically acceptable salt thereof, wherein the crystalline form of Formula (I) comprises Form H as characterized by an X-ray powder diffraction pattern comprising peaks at 8.8±0.3°2θ, 3.4±0.3°2θ, and 21.4±0.3°2θ. In some embodiments, the X-ray powder diffraction pattern further comprises at least one, at least two, at least three, or at least four peaks selected from 17.9±0.3°2θ, 14.5±0.3°2θ, 12.7±0.3°2θ, and 8.7±0.3°2θ. In some embodiments, the X-ray powder diffraction pattern further comprises at least one, at least two, or at least three peaks selected from 14.8±0.3°2θ, 12.8±0.3°2θ, and 21.2±0.3°2θ.

Aspects of the present disclosure provide a composition comprising a crystalline form of Formula (I):

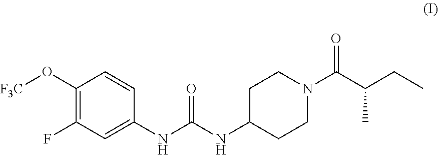 [SMILES: CC[C@H](C)C(=O)N1CCC(NC(=O)Nc2ccc(OC(F)(F)F)c(F)c2)CC1]

or a pharmaceutically acceptable salt thereof, wherein the crystalline form of Formula (I) comprises Form I as characterized by an X-ray powder diffraction pattern comprising peaks at 12.0±0.3°2θ, 12.3±0.3°2θ, and 3.2±0.3°2θ. In some embodiments, the X-ray powder diffraction pattern further comprises at least one, at least two, or at least three peaks selected from 14.5±0.3°2θ, 18.1±0.3°2θ, and 13.4±0.3°2θ. In some embodiments, the X-ray powder diffraction pattern further comprises at least one, at least two, or at least three peaks selected from 18.6±0.3°2θ, 24.8±0.3°2θ, and 19.1±0.3°2θ.

Aspects of the present disclosure provide a composition comprising a crystalline form of Formula (I):

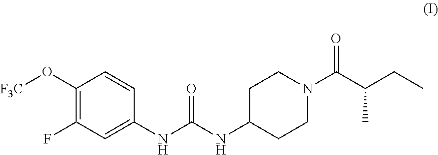 [SMILES: CC[C@H](C)C(=O)N1CCC(NC(=O)Nc2ccc(OC(F)(F)F)c(F)c2)CC1]

or a pharmaceutically acceptable salt thereof, wherein the crystalline form of Formula (I) comprises Form J as characterized by an X-ray powder diffraction pattern comprising peaks at 15.5±0.3°2θ, 15.7±0.3°2θ, and 17.6±0.3°2θ. In some embodiments, the X-ray powder diffraction pattern further comprises at least one, at least two, or at least three peaks selected from 15.1±0.3°2θ, 11.4±0.3°2θ, and 15.0±0.3°2θ. In some embodiments, the X-ray powder diffraction pattern further comprises at least one, at least two, or at least three peaks selected from 3.4±0.3°2θ, 20.2±0.3°2θ, and 21.0±0.3°2θ.

Aspects of the present disclosure provide a composition comprising a crystalline form of Formula (I):

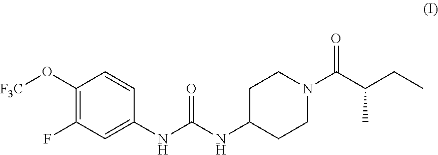 [SMILES: CC[C@H](C)C(=O)N1CCC(NC(=O)Nc2ccc(OC(F)(F)F)c(F)c2)CC1]

or a pharmaceutically acceptable salt thereof, wherein the crystalline form of Formula (I) comprises Form K as characterized by an X-ray powder diffraction pattern comprising peaks at 5.3±0.3°2θ, 14.5±0.3°2θ, and 7.3±0.3°2θ. In some embodiments, the X-ray powder diffraction pattern further comprises at least one, at least two, at least three, at least four, or at least five peaks selected from 20.9±0.3°2θ, 3.4±0.3°2θ, 21.1±0.3°2θ, 7.4±0.3°2θ, and 14.8±0.3°2θ. In some embodiments, the X-ray powder diffraction pattern further comprises a peak at 17.4±0.3°2θ.

Aspects of the present disclosure provide a composition comprising a crystalline form of Formula (I):

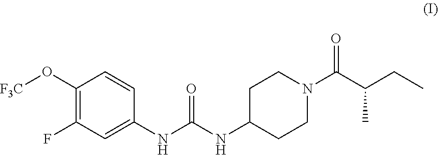 [SMILES: CC[C@H](C)C(=O)N1CCC(NC(=O)Nc2ccc(OC(F)(F)F)c(F)c2)CC1]

or a pharmaceutically acceptable salt thereof, wherein the crystalline form of Formula (I) comprises Form L as characterized by an X-ray powder diffraction pattern comprising peaks at 8.9±0.3°2θ, 3.4±0.3°2θ, and 18.3±0.3°2θ. In some embodiments, the X-ray powder diffraction pattern further comprises at least one, at least two, or at least three peaks selected from 14.4±0.3°2θ, 21.9±0.3°2θ, and 18.0±0.3°2θ. In some embodiments, the X-ray powder diffraction pattern further comprises at least one, at least two, at least three, at least four, or at least five peaks selected from 14.3±0.3°2θ, 13.2±0.3°2θ, 20.0±0.3°2θ, 19.3±0.3°2θ, and 14.9±0.3°2θ.

Aspects of the present disclosure provide a composition comprising an amorphous form of Formula (I):

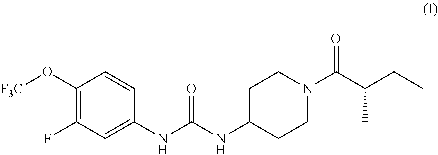 [SMILES: CC[C@H](C)C(=O)N1CCC(NC(=O)Nc2ccc(OC(F)(F)F)c(F)c2)CC1]

or a pharmaceutically acceptable salt thereof.

Aspects of the present disclosure provide a method of generating an amorphous form of Formula (I):

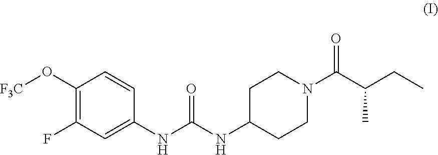 [SMILES: CC[C@H](C)C(=O)N1CCC(NC(=O)Nc2ccc(OC(F)(F)F)c(F)c2)CC1]

comprising converting a non-amorphous form of Formula (I) to the amorphous form of Formula (I). In some embodiments, the converting comprises an amorphous solid dispersion method. In some embodiments, the amorphous solid dispersion method comprises hot-melt extrusion or spray drying. In some embodiments, the non-amorphous form of Formula (I) comprises a crystalline form of Formula (I). In some embodiments, the crystalline form of Formula (I) is any one of Forms A-L. In some embodiments, the crystalline form of Formula (I) is Form D.

In some embodiments, at least about 20%, at least about 30%, at least about 40%, at least about 50%, at least about 60%, at least about 70%, at least about 80%, at least about 90%, or at least about 95% of Formula (I) is amorphous. In some embodiments, at most about 50%, at most about 45%, at most about 40%, at most about 35%, at most about 30%, at most about 25%, at most about 20%, at most about 15%, at most about 10%, at most about 5%, at most about 4%, at most about 3%, at most about 2%, at most about 1%, or at most about 0.5% of Formula (I) is amorphous.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### DESCRIPTION OF THE FIGURES

The novel features of the invention are set forth with particularity in the appended claims. A better understanding of the features and advantages of the present disclosure will be obtained by reference to the following detailed description that sets forth illustrative embodiments, in which the principles of the invention are utilized, and the accompanying drawings of which:

FIG. 1 Panel A provides a representative Xray powder diffraction spectrum (XRD) of Form A of Formula (I). FIG. 1 Panel B provides a representative XRD of Form B of Formula (I). FIG. 1 Panel C provides a representative XRD of Form C of Formula (I). FIG. 1 Panel D provides a representative XRD of Form D of Formula (I). FIG. 1 Panel E provides a representative XRD of Form E of Formula (I). FIG. 1 Panel F provides a representative XRD of Form F of Formula (I). FIG. 1 Panel G provides a representative XRD of Form G of Formula (I). FIG. 1 Panel H provides a representative XRD of Form H of Formula (I). FIG. 1 Panel I provides a representative XRD of Form I of Formula (I). FIG. 1 Panel J provides a representative XRD of Form J of Formula (I). FIG. 1 Panel K provides a representative XRD of Form K of Formula (I). FIG. 1 Panel L provides a representative XRD of Form L of Formula (I).

FIG. 2 provides a representative differential scanning calorimetry (DSC) thermogram of Form A of Formula (I).

FIG. 3 provides a representative DSC thermogram of Form B of Formula (I).

FIG. 4 provides a representative DSC thermogram of Form C of Formula (I).

FIG. 5 provides a representative DSC thermogram of Form D of Formula (I).

FIG. 6 provides a representative DSC thermogram of Form E of Formula (I).

FIG. 7 provides a representative DSC thermogram of Form F of Formula (I).

FIG. 8 provides a representative DSC thermogram of Form G of Formula (I).

FIG. 9 provides a representative DSC thermogram of Form H of Formula (I).

FIG. 10 provides a representative DSC thermogram of Form I of Formula (I).

FIG. 11 provides a representative DSC thermogram of Form J of Formula (I).

FIG. 12 provides a representative DSC thermogram of Form K of Formula (I).

FIG. 13 provides a representative DSC thermogram of Form L of Formula (I).

FIG. 14 provides a representative thermogravimetric analysis (TGA) thermogram of Form A of Formula (I).

FIG. 15 provides a representative TGA thermogram of Form B of Formula (I).

FIG. 16 provides a representative TGA thermogram of Form C of Formula (I).

FIG. 17 provides a representative TGA thermogram of Form D of Formula (I).

FIG. 18 provides a representative TGA thermogram of Form E of Formula (I).

FIG. 19 provides a representative TGA thermogram of Form F of Formula (I).

FIG. 20 provides a representative TGA thermogram of Form G of Formula (I).

FIG. 21 provides a representative TGA thermogram of Form H of Formula (I).

FIG. 22 provides a representative TGA thermogram of Form I of Formula (I).

FIG. 23 provides a representative TGA thermogram of Form J of Formula (I).

FIG. 24 provides a representative TGA thermogram of Form K of Formula (I).

FIG. 25 provides a representative TGA thermogram of Form L of Formula (I).

FIG. 26 provides a representative nuclear magnetic resonance (NMR) spectrum of Form A of Formula (I).

FIG. 27 provides a representative NMR spectrum of Form B of Formula (I).

FIG. 28 provides a representative NMR spectrum of Form C of Formula (I).

FIG. 29 provides a representative NMR spectrum of Form D of Formula (I).

FIG. 30 provides a representative NMR spectrum of Form E of Formula (I).

FIG. 31 provides a representative NMR spectrum of Form F of Formula (I).

FIG. 32 provides a representative NMR spectrum of Form G of Formula (I).

FIG. 33 provides a representative NMR spectrum of Form H of Formula (I).

FIG. 34 provides a representative NMR spectrum of Form I of Formula (I).

FIG. 35 provides a representative NMR spectrum of Form J of Formula (I).

FIG. 36 provides a representative NMR spectrum of Form K of Formula (I).

FIG. 37 provides a representative NMR spectrum of Form L of Formula (I).

FIG. 38 provides a calibration curve at 254 nm for polarized optical microscopy analysis.

FIG. 39 shows XRD spectra of Form B of Formula (I) from ten (top) and hundred (bottom) milligram-scale crystallizations.

FIG. 40 shows XRD spectra of Form A of Formula (I) from ten (top) and hundred (bottom) milligram-scale crystallizations.

FIG. 41 shows XRD spectra of Form L of Formula (I) from ten (top) and hundred (bottom) milligram-scale crystallizations.

FIG. 42 shows XRD spectra of Form A of Formula (I) from ten (top) and hundred (bottom) milligram-scale crystallizations.

FIG. 43 shows XRD spectra of Form D of Formula (I) from ten (top) and hundred (bottom) milligram-scale crystallizations.

FIG. 44 shows XRD spectra of Form H of Formula (I) from ten (top) and hundred (bottom) milligram-scale crystallizations.

FIG. 45 shows XRD spectra of Form L of Formula (I) from ten (top) and hundred (bottom) milligram-scale crystallizations.

FIG. 46 provides a DSC thermogram of Form B of Formula (I).

FIG. 47 provides a DSC thermogram of Form A of Formula (I).

FIG. 48 provides a DSC thermogram of Form L of Formula (I).

FIG. 49 provides a DSC thermogram of Form A of Formula (I).

FIG. 50 provides a DSC thermogram of Form D of Formula (I).

FIG. 51 provides a DSC thermogram of Form H of Formula (I).

FIG. 52 provides a DSC thermogram of Form L of Formula (I).

FIG. 53 provides a DSC thermogram of Form B of Formula (I).

FIG. 54 provides a DSC thermogram of Form A of Formula (I).

FIG. 55 provides a DSC thermogram of Form L of Formula (I).

FIG. 56 provides a DSC thermogram of Form A of Formula (I).

FIG. 57 provides a DSC thermogram of Form D of Formula (I).

FIG. 58 provides a DSC thermogram of Form H of Formula (I).

FIG. 59 provides a DSC thermogram of Form L of Formula (I).

FIG. 60 provides XRD spectra of Form I of Formula (I).

FIG. 61 provides XRD spectra of Form G of Formula (I).

FIG. 62 provides XRD spectra of Form F of Formula (I).

FIG. 63 provides XRD spectra of Form E of Formula (I).

FIG. 64 provides representative XRD spectra of Form C of Formula (I) (top), Formula (I) following 1 day of incubation in water (middle), and Formula (I) following 7 days of incubation in water (bottom).

FIG. 65 provides representative XRD spectra of Form K of Formula (I) (top), Formula (I) following 1 day of incubation in MTBE (middle), and Formula (I) following 7 days of incubation in MTBE (bottom).

FIG. 66 provides representative XRD spectra of Form D of Formula (I) (top), Form E of Formula (I) (second from top), Formula (I) following 1 day of incubation in n-heptane (second from bottom), and Formula (I) following 7 days of incubation in n-heptane (bottom).

FIG. 67 provides representative XRD spectra of Form A of Formula (I) (top), Form D of Formula (I) (second from top), Formula (I) following one day of incubation in 70° C. n-heptane (second from bottom), and Formula (I) following seven days incubation in 70° C. n-heptane (bottom).

FIG. 68 provides representative XRD spectra of Form D of Formula (I) (top), Form E of Formula (I) (second from top), Formula (I) following one day of incubation in 70° C. n-heptane (second from bottom), and Formula (I) following seven days incubation in 70° C. n-heptane (bottom).

FIG. 69 provides representative XRD spectra of Form A of Formula (I) (top), Form E of Formula (I) (second from top), Formula (I) following one day of incubation in n-heptane (second from bottom), and Formula (I) following seven days incubation in n-heptane (bottom).

FIG. 70 provides representative XRD spectra of Form D of Formula (I) (top), Formula (I) following one day of incubation in n-heptane (middle), and Formula (I) following seven days incubation in n-heptane (bottom).

FIG. 71 provides representative XRD spectra of Form E of Formula (I) (top), Formula (I) following one day of incubation in n-heptane (middle), and Formula (I) following seven days incubation in n-heptane (bottom).

FIG. 72 provides representative XRD spectra of Form C of Formula (I) (top), Form E of Formula (I) (second from top), Formula (I) following one day of incubation in n-heptane (second from bottom), and Formula (I) following seven days incubation in n-heptane (bottom).

FIG. 73 provides representative XRD spectra of Form A of Formula (I) (top), Form A of Formula (I) following 1 day of incubation at 60° C. (middle), and Form A of Formula (I) following 7 days of incubation at 60° C. (bottom).

FIG. 74 provides representative XRD spectra of Form B of Formula (I) (top), Form D of Formula (I) (second from top), Form D of Formula (I) following 1 day of incubation at 60° C. (second from bottom), and Form D of Formula (I) following 7 days of incubation at 60° C. (bottom).

FIG. 75 provides representative XRD spectra of Form D of Formula (I) (top), Form D of Formula (I) following 1 day of incubation at 60° C. (middle), and Form D of Formula (I) following 7 days of incubation at 60° C. (bottom).

FIG. 76 provides representative XRD spectra of Form E of Formula (I) (top), Form E of Formula (I) following 1 day of incubation at 60° C. (middle), and Form E of Formula (I) following 7 days of incubation at 60° C. (bottom).

FIG. 77 provides representative XRD spectra of Form F of Formula (I) (top), Form F of Formula (I) following 1 day of incubation at 60° C. (middle), and Form F of Formula (I) following 7 days of incubation at 60° C. (bottom).

FIG. 78 provides representative XRD spectra of Form G of Formula (I) (top), Form L of Formula (I) (second from top), Form G of Formula (I) following 1 day of incubation at 60° C. (middle), and Form G of Formula (I) following 7 days of incubation at 60° C. (bottom).

FIG. 79 provides representative XRD spectra of Form I of Formula (I) (top), Form I of Formula (I) following 1 day of incubation at 60° C. (middle), and Form I of Formula (I) following 7 days of incubation at 60° C. (bottom).

FIG. 80 provides representative XRD spectra of Form A of Formula (I) (top), Form C of Formula (I) (second from top), starting Form A of Formula (I) (middle), Form A of Formula (I) following 1 day of incubation at 60° C. (second from bottom), and Form A of Formula (I) following 7 days of incubation at 60° C. (bottom).

FIG. 81 provides an HPLC chromatogram of Form D of Formula (I) prior to high-temperature incubation.

FIG. 82 provides an H PLC chromatogram of Form D of Formula (I) following 1 day of 60° C. incubation.

FIG. 83 provides an H PLC chromatogram of Form D of Formula (I) following 7 days of 60° C. incubation.

FIG. 84 provides XRD spectra of Form D of Formula (I), with the top spectrum corresponding to a representative Form D pattern, the second spectrum from the top corresponding to Form D of Formula (I) prior to humidity exposure, the second spectrum from the bottom corresponding to Form D of Formula (I) following 4 days of humidity exposure, and the bottom spectrum corresponding to Form D of Formula (I) following 14 days of humidity exposure.

FIG. 85 provides XRD spectra of Form C of Formula (I) (top), Form D of Formula (I) (second from top), a Form C and D mixture following incubation in PEG300 containing 75% water (third from top), a Form C and D mixture following incubation in PEG300 containing 50% water (middle), a Form C and D mixture following incubation in PEG300 containing 25% water (third from bottom), a Form C and D mixture following incubation in PEG300 containing 10% water (second from bottom), and a Form C and D mixture following incubation in PEG300 containing 5% water (bottom).

FIG. 86 provides a representative H PLC chromatogram of Formula (I) following incubation in diethylene glycol monoethyl ether.

FIG. 87 provides representative XRD spectra of Form A of Formula (I) (top), Form C of Formula (I) (second from top), and three batches of Formula (I) (bottom three spectra).

FIG. 88 provides a DSC thermogram of Formula (I).

FIG. 89 provides a TGA thermogram of Formula (I).

FIG. 90 provides a modulated DSC thermogram of Formula (I).

FIG. 91 provides two polarized-light microscopic images of Formula (I).

FIG. 92 provides dynamic vapor sorption plots of Formula (I).

FIG. 93 provides a DSC thermogram of Formula (I).

FIG. 94 provides a TGA thermogram of Formula (I).

FIG. 95 provides two polarized-light microscopic images of Formula (I).

FIG. 96 provides dynamic vapor sorption plots of Formula (I).

FIG. 97 provides a DSC thermogram of Formula (I).

FIG. 98 provides XRD spectra of Form D of Formula (I) (top) and three separate preparations of Formula (I) (bottom three spectra).

FIG. 99 provides XRD spectra of multiple Formula (I) polymorphs.

FIG. 100 provides XRD spectra of Form D of Formula (I) (top) and Formula (I) sample generated from a crystal-seeding experiment.

FIG. 101 provides XRD spectra of Formula (I) with 2.5% (top), 5% (second from top), 7.5% (third from top), 10% (middle), and 15% (second from bottom) Form C mixed into Form D, along with representative spectra of Form D (second from bottom) and Form C (bottom).

FIG. 102 provides zoomed-in versions of the XRD spectra from FIG. 101.

FIG. 103 provides XRD data of unmilled Form D of Formula (I) (top and middle) and of milled Form D of Formula (I) (bottom).

FIG. 104 provides a DSC thermogram of milled Form D of Formula (I).

FIG. 105 provides thermograms of Form D of Formula (I) in micronized form (top), Form D of Formula (I) following 5 minutes of grinding (second from top), Form D of Formula (I) following 10 minutes of grinding (second from bottom), and Form D of Formula (I) following 15 minutes of grinding (bottom).

FIG. 106 provides optical microscope images of unmilled Form D of Formula (I).

FIG. 107 provides optical microscope images of milled Form D of Formula (I).

FIG. 108 provides particle size data for unmilled Form D of Formula (I) utilizing 2.5 bar pressure.

FIG. 109 provides particle size data for unmilled Form D of Formula (I) utilizing 3.0 bar pressure.

FIG. 110 provides particle size data for unmilled Form D of Formula (I) utilizing 3.5 bar pressure.

FIG. 111 provides particle size data for milled Form D of Formula (I) utilizing 2.5 bar pressure.

FIG. 112 provides particle size data for milled Form D of Formula (I) utilizing 3.0 bar pressure.

FIG. 113 provides particle size data for milled Form D of Formula (I) utilizing 3.5 bar pressure. FIG. 114 provides particle size data for unmilled Form D of Formula (I) utilizing 2.5 bar pressure and a high energy venturi.

FIG. 115 provides particle size data for unmilled Form D of Formula (I) utilizing 3.0 bar pressure and a high energy venturi.

FIG. 116 provides particle size data for unmilled Form D of Formula (I) utilizing 3.5 bar pressure and a high energy venturi.

FIG. 117 provides particle size data for milled Form D of Formula (I) utilizing 2.5 bar pressure and a high energy venturi.

FIG. 118 provides particle size data for milled Form D of Formula (I) utilizing 3.0 bar pressure and a high energy venturi.

FIG. 119 provides particle size data for milled Form D of Formula (I) utilizing 3.5 bar pressure and a high energy venturi.

FIG. 120 provides XRD spectra of Form C of Formula (I) (top) and amorphous Formula (I) (bottom).

FIG. 121 provides a DSC thermogram of amorphous Formula (I).

FIGS. 122-123 summarize Formula (I) sodium lauryl sulfate (SLS) kinetic solubility data over 180 and 90 minutes, respectively.

FIG. 124 provides XRD spectra of Form C of Formula (I) (top), Form (D) of Formula (I) (second from top), unmilled Form D of Formula (I) following 5 minutes incubation in SLS (third from top), unmilled Form D of Formula (I) following 60 minutes incubation in SLS (third from bottom), unmilled Form D of Formula (I) following 90 minutes of incubation in SLS (second from bottom), and unmilled Form D of Formula (I) following 180 minutes of incubation in SLS.

FIG. 125 provides XRD spectra of Form C of Formula (I) (top), Form (D) of Formula (I) (second from top), milled Form D of Formula (I) following 5 minutes incubation in SLS (third from top), milled Form D of Formula (I) following 60 minutes incubation in SLS (third from bottom), milled Form D of Formula (I) following 90 minutes of incubation in SLS (second from bottom), and milled Form D of Formula (I) following 180 minutes of incubation in SLS.

FIG. 126 provides XRD spectra of Form C of Formula (I) (top), Form (D) of Formula (I) (second from top), amorphous Formula (I) following 5 minutes incubation in SLS (third from top), amorphous Formula (I) following 60 minutes incubation in SLS (third from bottom), amorphous Formula (I) following 90 minutes of incubation in SLS (second from bottom), and amorphous Formula (I) following 180 minutes of incubation in SLS.

FIG. 127 provides a DSC thermogram of Formula (I).

FIG. 128 provides a ¹H NMR spectrum of Formula (I).

FIG. 129 provides a ¹³C NMR spectrum of Formula (I).

FIG. 130 provides a ¹⁹F NMR spectrum of Formula (I).

FIGS. 131A-L provide representative X ray powder diffraction spectra of various polymorphs of Formula (I). FIG. 131A provides a representative X ray powder diffraction spectrum (XRD) of Form A of Formula (I). FIG. 131B provides a representative XRD of Form B of Formula (I). FIG. 131C provides a representative XRD of Form C of Formula (I). FIG. 131D provides a representative XRD of Form D of Formula (I). FIG. 131E provides a representative XRD of Form E of Formula (I). FIG. 131F provides a representative XRD of Form F of Formula (I). FIG. 131G provides a representative XRD of Form G of Formula (I). FIG. 131H provides a representative XRD of Form H of Formula (I). FIG. 1311 provides a representative XRD of Form I of Formula (I). FIG. 131J provides a representative XRD of Form J of Formula (I). FIG. 131K provides a representative XRD of Form K of Formula (I). FIG. 131L provides a representative XRD of Form L of Formula (I).

### DETAILED DESCRIPTION

Soluble epoxide hydrolase (sEH) is central to many forms of lipid metabolism, and may participate in the degradation of cytochrome P450 oxidized xenobiotics. While the sEH active site is selective for hydrophobic species (often mimetic of its native lipid substrates), sEH is predominantly localized within cytosol and cytosolic peroxisomal fractions, and therefore is often only targetable by aqueous species. Accordingly, sEH inhibitor delivery is often a major barrier to sEH-regulation. As a means for addressing this challenge, the present disclosure provides a range of polymorphic forms of an sEH inhibitor (Formula (I)) with physical properties well suited for formulation and therapeutic use.

Compounds are described using standard nomenclature. Unless otherwise defined, all technical and scientific terms used herein have the same meaning as is commonly understood by one of skill in the art to which this invention belongs.

As used herein, the terms “a,” “an,” and “the” include plural reference unless the context dictates otherwise.

As used herein and in the claims, the terms “comprising,” “containing,” and “including” are inclusive, open-ended and do not exclude additional unrecited elements, compositional components or method steps. Accordingly, the terms “comprising” and “including” encompass the comparably more restrictive terms “consisting of” and “consisting essentially of.”

As used herein, “soluble epoxide hydrolase” (“sEH”) refers to enzymes which in endothelial, smooth muscle and other cell types convert EETs to dihydroxy derivatives called dihydroxyeicosatrienoic acids (“DH ETs”). The cloning and sequence of the murine sEH is set forth in Grant et al., J. Biol. Chem. 268(23): 17628-17633 (1993). The cloning, sequence, and accession numbers of the human sEH sequence are set forth in Beetham et ah, Arch. Biochem. Biophys. 305(1): 197-201 (1993). The amino acid sequence of human sEH is also set forth as SEQ ID NO:2 of U.S. Pat. No. 5,445,956; the nucleic acid sequence encoding the human sEH is set forth as nucleotides 42-1703 of SEQ ID NO: 1 of that patent. The evolution and nomenclature of the gene is discussed in Beetham et al., DNA Cell Biol. 14(1):61-71 (1995). Soluble epoxide hydrolase represents a single highly conserved gene product with over 90% homology between rodent and human (A rand et al., F E B S Lett., 338:251-256 (1994)).

As used herein, the terms “active pharmaceutical ingredient”, “active ingredient”, “API,” “drug,” “active,” “actives” and “therapeutic agent” may be used interchangeably to refer to a pharmaceutically active compound(s) in a pharmaceutical composition. This can be in contrast to other ingredients in the compositions, such as excipients, which are substantially or completely pharmaceutically inert. Suitable APIs in accordance with the present disclosure include those for which there are or are likely to be patient compliance issues for treating a certain disease, condition, or disorder. The therapeutic agent as used herein includes the active compound and its salts, prodrugs, and metabolites.

As used herein, the term “drug” denotes a compound intended for use in diagnosis, cure, mitigation, treatment, and/or prevention of disease in humans or other animals.

As used herein, the term “subject” includes animals such as mammals, including, but not limited to, primates (e.g., humans), cows, sheep, goats, horses, dogs, cats, rabbits, rats, mice and the like. In some embodiments, the subject is a human.

As used herein, the terms “treat”, “treating” and “treatment” can denote methods of alleviating or abrogating a disease or its attendant symptoms.

References to “a compound” or “compounds” throughout this application, such as compounds of Formula (I), Formula (II), Formula (III) and Formula (IV), include the polymorphic, amorphic, salt, free base, acid salt, co-crystal, and solvate forms of those formulas and/or compounds unless further specified. Thus, the appearances of the phrases “a compound”, “a compound of Formula (I)”, “compounds of Formula (I)”, etc. include polymorphic forms of the compound of Formula (I), such as Forms A-L of the compounds of Formula (I) as further disclosed herein.

“Crystalline form” and “polymorph” may be used interchangeably herein, and are meant to include all crystalline forms of the compound, including, for example, polymorphs and pseudopolymorphs.

The term “Form” can be taken to encompass the terms “crystalline form” and “polymorph,” as well as other descriptions of physical state (e.g., “solvated,” “amorphous,” etc.). The term “Form” can denote a salt, solvate, hydrate, unsolvated polymorph (including anhydrates), conformational polymorph, and amorphous form, as well as a mixture thereof, unless a particular form or physical characteristic is otherwise specified.

The term “substantially as shown in” when referring, for example, to an X-ray powder diffraction (XRPD) pattern, includes a pattern that is not necessarily identical to those depicted herein, but that falls within the limits of experimental error or deviations when considered by one of ordinary skill in the art.

The relative intensities of XRPD peaks can vary, depending upon the particle size, the sample preparation technique, the sample mounting procedure and the particular instrument employed.

Moreover, instrument variation and other factors can affect the two theta (20) values.

Accordingly, when a specified two theta angle is provided, it is to be understood that the specified two theta angle can vary by the specified value 0.50, such as 0.40, 0.30, 0.20, or 0.10.

As used herein, the term “major peak” refers to an X RPD peak with a relative intensity greater than 30%, such as greater than 35%. Relative intensity is calculated as a ratio of the peak intensity of the peak of interest versus the peak intensity of the largest peak in the XRPD pattern.

Compounds of the present disclosure include crystalline and amorphous forms of those compounds, including, for example, polymorphs, pseudopolymorphs, salts, solvates, hydrates, unsolvated polymorphs (including anhydrates), conformational polymorphs, and amorphous forms of the compounds, as well as mixtures thereof.

All compounds disclosed herein are further understood to include all possible isotopes of atoms occurring in the compounds. Isotopes include those atoms having the same atomic number but different mass numbers. By way of example, and without limitation, isotopes of hydrogen include tritium and deuterium and isotopes of carbon include ¹²C, ³C and ¹⁴C.

As used herein, the term “dosage form” can denote the form in which a compound or composition of the present disclosure are delivered to a patient, and includes physical form (e.g., microcrystalline, micellular, etc.) and characteristics (e.g., powder pressed into a pill form).

As used herein, the term “combination therapy” can refer to the use of a compound, composition, or therapy described herein in combination with one or more additional compounds, compositions, or therapy (e.g., radiotherapy). Two or more compounds, compositions, therapies, or combinations thereof can be co-administered, or provided in different administration schedules and/or dosage forms.

As used herein, the term “pharmaceutical composition” can denote a combination of an active agent and a pharmaceutically acceptable excipient (e.g., a carrier), rendering the compositions suitable or enhancing their suitability for diagnostic or therapeutic uses in vitro, in vivo, and/or ex vivo.

A s used herein, the term “compound” is intended to encompass not only the specified molecular entity but also its pharmaceutically acceptable, pharmacologically active derivatives, including, but not limited to, salts, prodrug conjugates such as esters and amides, metabolites and the like.

As used herein, the term “composition” encompasses a product comprising the specified ingredients in the specified amounts, as well as any product which results, directly or indirectly, from combination of the specified ingredients in the specified amounts.

As used herein, “pharmaceutically acceptable” denotes that the carrier, diluent or excipient must be compatible with the other ingredients of the formulation and not deleterious to the recipient thereof.

As used herein, the term “alkyl” refers to a saturated hydrocarbon radical which may be straight-chain or branched-chain (for example, ethyl, isopropyl, t-amyl, or 2,5-dimethylhexyl). This definition applies both when the term is used alone and when it is used as part of a compound term, such as “aralkyl,” “alkylamino” and similar terms. In some embodiments, alkyl groups are those containing 1 to 24 carbon atoms. All numerical ranges in this specification and claims are intended to be inclusive of their upper and lower limits. Lower alkyl refers to those alkyl groups having 1 to 4 carbon atoms. Additionally, the alkyl and heteroalkyl groups may be attached to other moieties at any position on the alkyl or heteroalkyl radical which would otherwise be occupied by a hydrogen atom (such as, for example, 2-pentyl, 2-methylpent-1-yl and 2-propyloxy). Divalent alkyl groups may be referred to as “alkylene”, and divalent heteroalkyl groups may be referred to as “heteroalkylene” such as those groups used as linkers in the present invention. The alkyl, alkylene, and heteroalkyl moieties may also be optionally substituted with halogen atoms, or other groups such as oxo, cyano, nitro, alkyl, alkylamino, carboxyl, hydroxyl, alkoxy, aryloxy, and the like.

As used herein, the terms “cycloalkyl” and “cycloalkenyl” refer to a saturated hydrocarbon ring and includes bicyclic and polycyclic rings. Similarly, cycloalkyl and cycloalkenyl groups having a heteroatom (e.g. N, O or S) in place of a carbon ring atom may be referred to as “heterocycloalkyl” and heterocycloalkylene,” respectively. Examples of cycloalkyl and heteroaryl groups are, for example, cyclohexyl, norbornyl, adamantly, morpholinyl, thiomorpholinyl, dioxothiomorpholinyl, and the like. The cycloalkyl and heterocycloalkyl moieties may also be optionally substituted with halogen atoms, or other groups such as nitro, alkyl, alkylamino, carboxyl, alkoxy, aryloxy and the like. In some embodiments, cycloalkyl and cycloalkenyl moieties are those having 3 to 12 carbon atoms in the ring (e.g., cyclohexyl, cyclooctyl, norbornyl, adamantyl, and the like). In some embodiments, heterocycloalkyl and heterocycloalkylene moieties are those having 1 to 3 hetero atoms in the ring (e.g., morpholinyl, thiomorpholinyl, dioxothiomorpholinyl, piperidinyl and the like). Additionally, the term “(cycloalkyl)alkyl” refers to a group having a cycloalkyl moiety attached to an alkyl moiety. Examples are cyclohexylmethyl, cyclohexylethyl and cyclopentylpropyl.

As used herein, the term “alkenyl” refers to an alkyl group as described above which contains one or more sites of unsaturation that is a double bond. Similarly, the term “alkynyl” as used herein refers to an alkyl group as described above which contains one or more sites of unsaturation that is a triple bond.

As used herein, the term “alkoxy” refers to an alkyl radical as described above which also bears an oxygen substituent which is capable of covalent attachment to another hydrocarbon radical (such as, for example, methoxy, ethoxy, aryloxy and t-butoxy).

As used herein, the term “aryl” refers to an aromatic carbocyclic substituent which may be a single ring or multiple rings which are fused together, linked covalently or linked to a common group such as an ethylene or methylene moiety. Similarly, aryl groups having a heteroatom (e.g. N, O or S) in place of a carbon ring atom are referred to as “heteroaryl”. Examples of aryl and heteroaryl groups are, for example, phenyl, naphthyl, biphenyl, diphenylmethyl, 2,2-diphenyl-1-ethyl, thienyl, pyridyl and quinoxalyl. The aryl and heteroaryl moieties may also be optionally substituted with halogen atoms, or other groups such as nitro, alkyl, alkylamino, carboxyl, alkoxy, phenoxy and the like. Additionally, the aryl and heteroaryl groups may be attached to other moieties at any position on the aryl or heteroaryl radical which would otherwise be occupied by a hydrogen atom (such as, for example, 2-pyridyl, 3-pyridyl and 4-pyridyl). Divalent aryl groups are “arylene”, and divalent heteroaryl groups are referred to as “heteroarylene” such as those groups used as linkers in the present invention.

As used herein, the terms “arylalkyl”, “arylalkenyl” and “aryloxyalkyl” refer to an aryl radical attached directly to an alkyl group, an alkenyl group, or an oxygen which is attached to an alkyl group, respectively. For brevity, aryl as part of a combined term as above, is meant to include heteroaryl as well.

As used herein, the terms “halo” or “halogen,” by themselves or as part of another substituent, mean, unless otherwise stated, a fluorine, chlorine, bromine, or iodine atom. Additionally, terms such as “haloalkyl,” are meant to include monohaloalkyl and polyhaloalkyl. For example, the term “C₁-C₆ haloalkyl” is mean to include trifluoromethyl, 2,2,2-trifluoroethyl, 4-chlorobutyl, 3-bromopropyl, and the like.

As used herein, the term “hetero” as used in a “heteroatom” refers to any atom other than carbon or hydrogen, e.g., nitrogen, oxygen, sulfur, phosphorus or silicon.

As used herein, the term “hetero” as used in a “hetero atom-containing alkyl group” (a “heteroalkyl” group) or a “heteroatom-containing aryl group” (a “heteroaryl” group) refers to a molecule, linkage or substituent in which one or more carbon atoms are replaced with an atom other than carbon, e.g., nitrogen, oxygen, sulfur, phosphorus or silicon, typically nitrogen, oxygen or sulfur or more that none non-carbon atom (e.g., sulfonamide). Similarly, the term “heteroalkyl” refers to an alkyl substituent that is heteroatom-containing, the term “heterocyclic” refers to a cyclic substituent that is heteroatom-containing, the terms “heteroaryl” and heteroaromatic” respectively refer to “aryl” and “aromatic” substituents that are heteroatom-containing, and the like. Examples of heteroalkyl groups include alkoxyaryl, alkylsulfanyl-substituted alkyl, N-alkylated amino alkyl, and the like. Examples of heteroaryl substituents include pyrrolyl, pyrrolidinyl, pyridinyl, quinolinyl, indolyl, pyrimidinyl, imidazolyl, 1,2,4-triazolyl, tetrazolyl, etc., and examples of heteroatom-containing alicyclic groups are pyrrolidino, morpholino, piperazino, piperidino, etc.

As used herein, the terms “hydrophobic radical” and “hydrophobic group” refer to a group which lowers the water solubility of a molecule. In some embodiments, hydrophobic radicals are groups containing at least 3 carbon atoms.

As used herein, the term “carboxylic acid analog” refers to a variety of groups having an acidic moiety that are capable of mimicking a carboxylic acid residue. Examples of such groups are sulfonic acids, sulfinic acids, phosphoric acids, phosphonic acids, phosphinic acids, sulfonamides, and heterocyclic moieties such as, for example, imidazoles, triazoles and tetrazoles.

A s used herein, the term “substituted” refers to the replacement of an atom or a group of atoms of a compound with another atom or group of atoms. For example, an atom or a group of atoms may be substituted with one or more of the following substituents or groups: halo, cyano, nitro, alkyl, alkylamino, hydroxyalkyl, haloalkyl, carboxyl, hydroxyl, alkoxy, alkoxyalkoxy, haloalkoxy, thioalkyl, aryl, aryloxy, cycloalkyl, cycloalkylalkyl, aryl, heteroaryl optionally substituted with 1 or more, preferably 1 to 3, substituents selected from halo, halo alkyl and alkyl, aralkyl, heteroaralkyl, alkenyl containing 1 to 2 double bonds, alkynyl containing 1 to 2 triple bonds, alk(en)(yn)yl groups, halo, cyano, hydroxy, haloalkyl and polyhaloalkyl, preferably halo lower alkyl, especially trifluoromethyl, formyl, alkylcarbonyl, arylcarbonyl that is optionally substituted with 1 or more, preferably 1 to 3, substituents selected from halo, halo alkyl and alkyl, heteroarylcarbonyl, carboxy, alkoxycarbonyl, aryloxycarbonyl, aminocarbonyl, alkylaminocarbonyl, dialkylaminocarbonyl, arylaminocarbonyl, diarylaminocarbonyl, aralkylaminocarbonyl, alkoxy, aryloxy, perfluoroalkoxy, alkenyloxy, alkynyloxy, arylalkoxy, aminoalkyl, alkylaminoalkyl, dialkylaminoalkyl, arylaminoalkyl, amino, alkylamino, dialkylamino, arylamino, alkylarylamino, alkylcarbonylamino, arylcarbonylamino, azido, nitro, mercapto, alkylthio, arylthio, perfluoroalkylthio, thiocyano, isothiocyano, alkylsulfinyl, alkylsulfonyl, arylsulfinyl, arylsulfonyl, aminosulfonyl, alkylaminosulfonyl, dialkylaminosulfonyl and arylaminosulfonyl. When the term “substituted” appears prior to a list of possible substituted groups, it is intended that the term apply to every member of that group.

The term “unsubstituted” refers to a native compound that lacks replacement of an atom or a group of atoms.

### Soluble Epoxide Hydrolase Inhibitor

The present disclosure provides the soluble epoxide hydrolase inhibitor (sEH) Formula (I) which is efficacious for treating sEH mediated diseases and disorders.

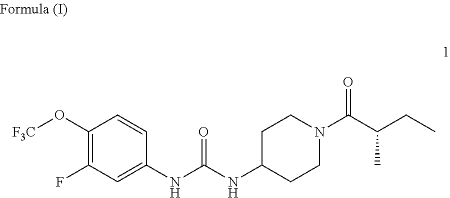 [SMILES: CC[C@H](C)C(=O)N1CCC(NC(=O)Nc2ccc(OC(F)(F)F)c(F)c2)CC1]

Soluble epoxide hydrolase is configured to bind a range of epoxidated fatty acid substrates, and can include a binding site with two hydrophobic pockets and a hydrophilic core which can include a bis-tyrosine substrate hydrogen bonding motif and an aspartic acid-histidine-aspartate salt bridge. Formula (I) effectively binds to these hydrophobic and hydrophilic regions, and has sufficient hydrophobicity to reach these regions by traversing hydrophobic sEH substrate access channels. Exemplifying these characteristics, Formula (I) exhibits an sEH-inhibitor constant (Ki) of less than 50 pM and a t_{1/2} for sEH inhibition of 22 minutes.

Formula (I) also has a relatively low aqueous solubility of 11 μg/mL, which can challenge formulation for cellular uptake and sEH colocalization. As sEH is primarily peroxisomal and cytosolic, effective Formula (I) delivery can require formulations which facilitate aqueous localization. To overcome this barrier, Formula (I) can be formulated and crystallized to enhance its activity and solubility.

### Formula (I) Polymorphic Forms

A surprising discovery disclosed herein is that Formula (I) can be prepared in multiple crystalline forms (hereinafter “Forms” or “Polymorphs”), each with unique solubilities and stabilities, and which are thus useful for tailoring formulations for particular treatments and means of delivery. Among the aspects of the present disclosure are polymorphic Forms A, B, C, D, E, F, G, H, I, J, K, and L of Formula (I). Each of these forms possesses unique physical structures and properties, including distinct solubilities and stabilities in various conditions and solvents. Forms A-L each differ from amorphous Formula (I) (e.g., as outlined in EXAMPLES 4-6), and thereby expand options for formulating Formula (I) beyond those which were previously available.

A composition can comprise a single, non-amorphous form of Formula (I). In some cases, a composition comprises at least 50%, at least 60%, at least 70%, at least 80%, at least 90%, at least 95%, at least 98%, at least 99%, or at least 99.5% of any one of Forms A-L by weight. A composition can comprise a mixture of non-amorphous forms of Formula (I), for example at least 50%, at least 60%, at least 70%, at least 80%, at least 90%, at least 95%, at least 98%, at least 99%, or at least 99.5% of two or more of Forms A-L by weight. In some cases, a composition comprises less than 30%, less than 20%, less than 15%, less than 10%, less than 5%, less than 2%, less than 1%, less than 0.5%, or less than 0.1% amorphous Formula (I) by weight.

Form C is a hydrate, differing from Forms A, B, and D-L, which are anhydrous or substantially anhydrous (e.g., comprise less than about 3% water by weight)]. In some cases, a composition with Formula (I) comprises at least 85%, at least 90%, at least 95%, at least 98%, or at least 99% anhydrous Formula (I) by weight. In some cases, Formula (I) (either a single form or a mixture of forms) comprises less than 5%, less than 4%, less than 3%, less than 2%, less than 1.5%, less than 1%, less than 0.5%, less than 0.25%, or less than 0.1% water by weight. In some cases, Formula (I) is at least 70%, at least 80%, at least 90%, at least 95%, at least 98%, at least 99%, at least 99.5%, or at least 99.5% Form A, B, D, E, F, G, H, I, J, K, L, or a combination thereof.

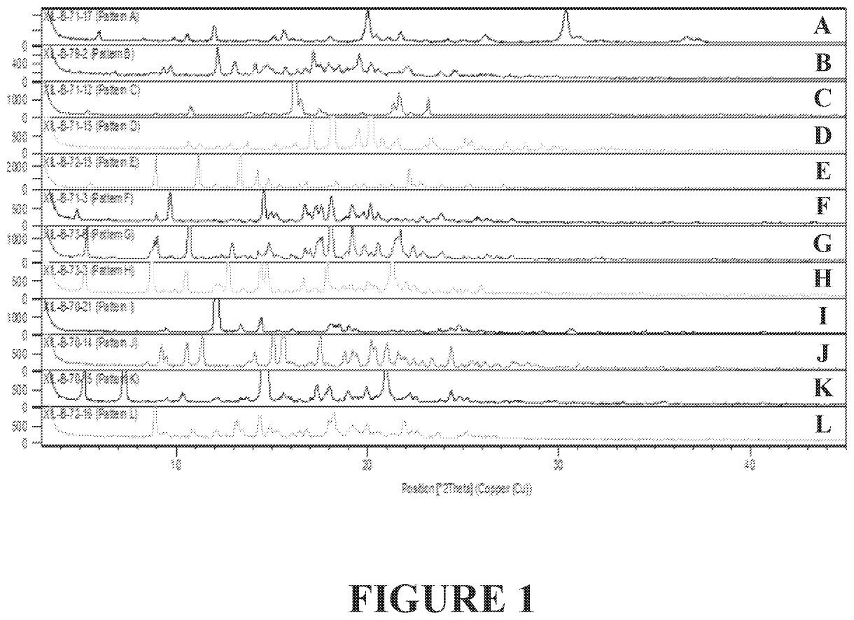

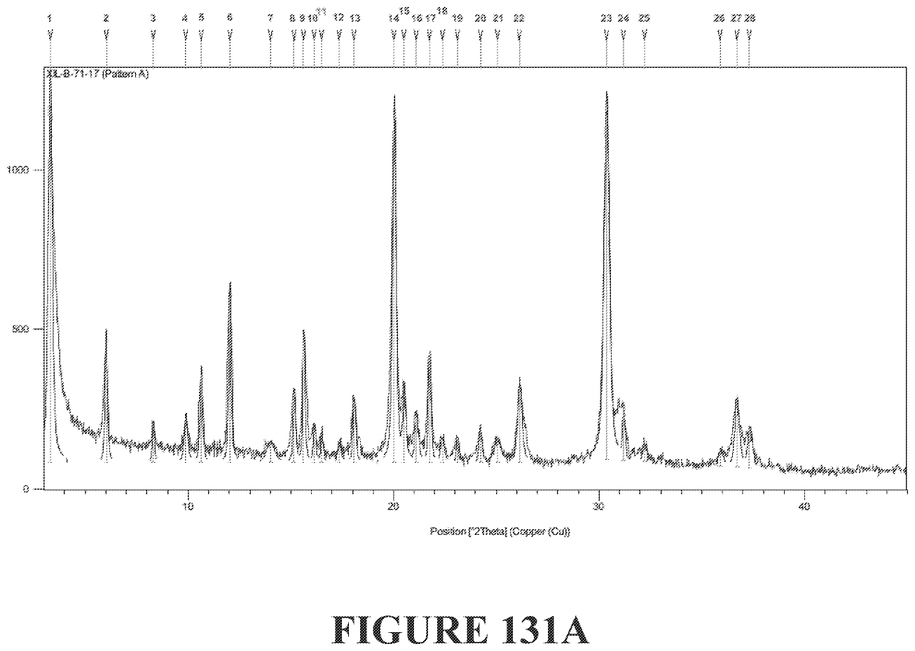

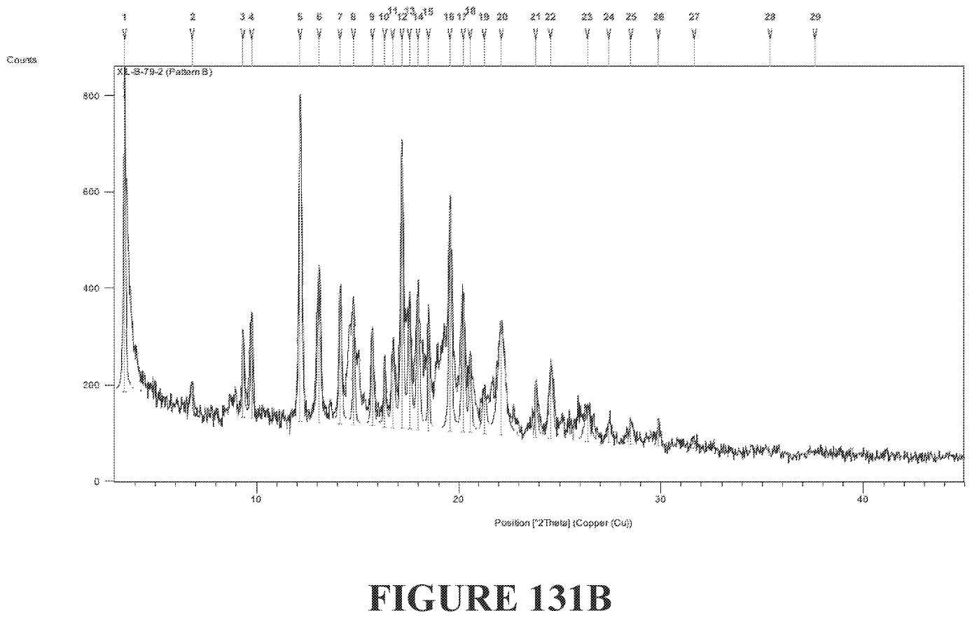

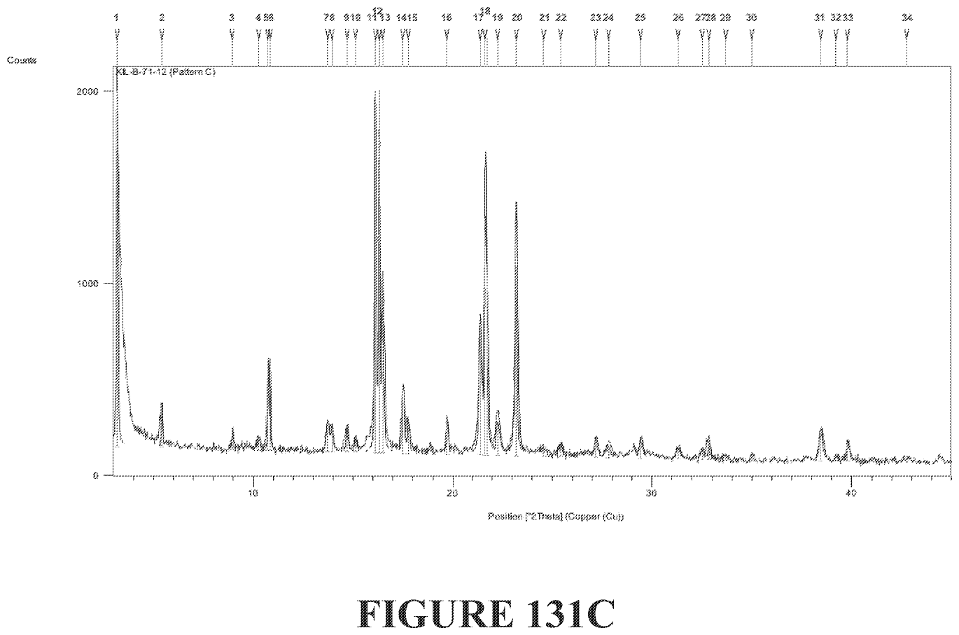

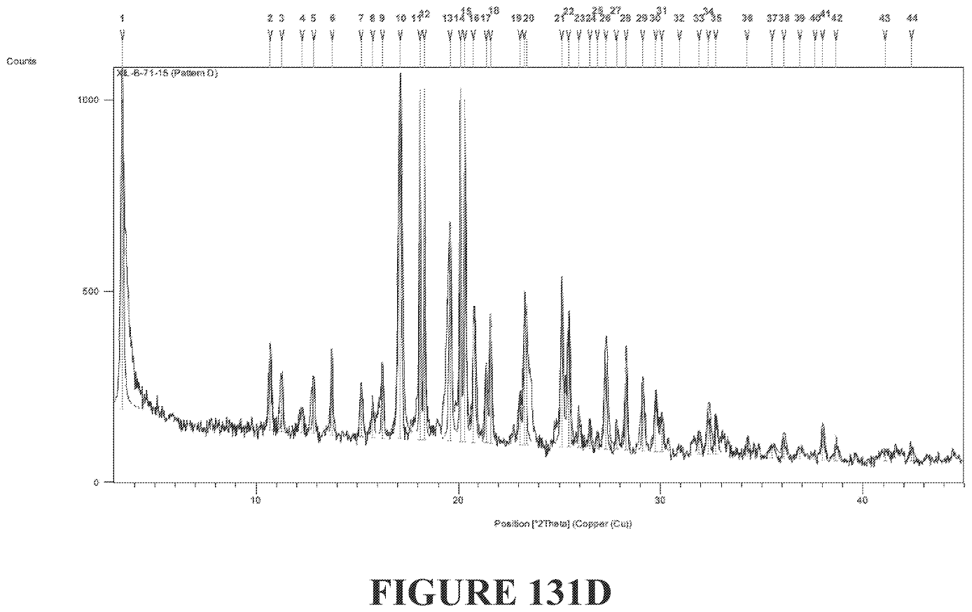

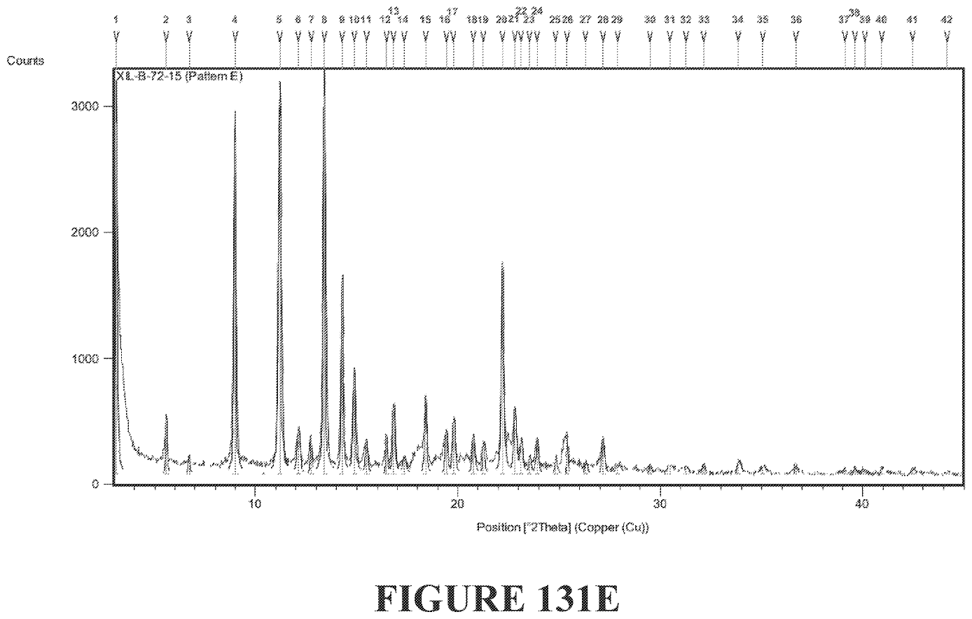

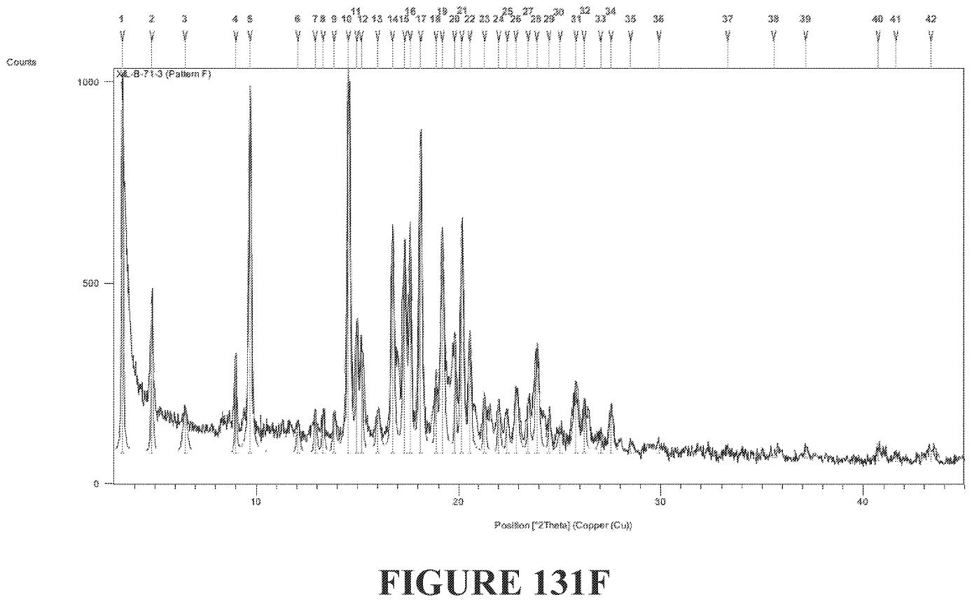

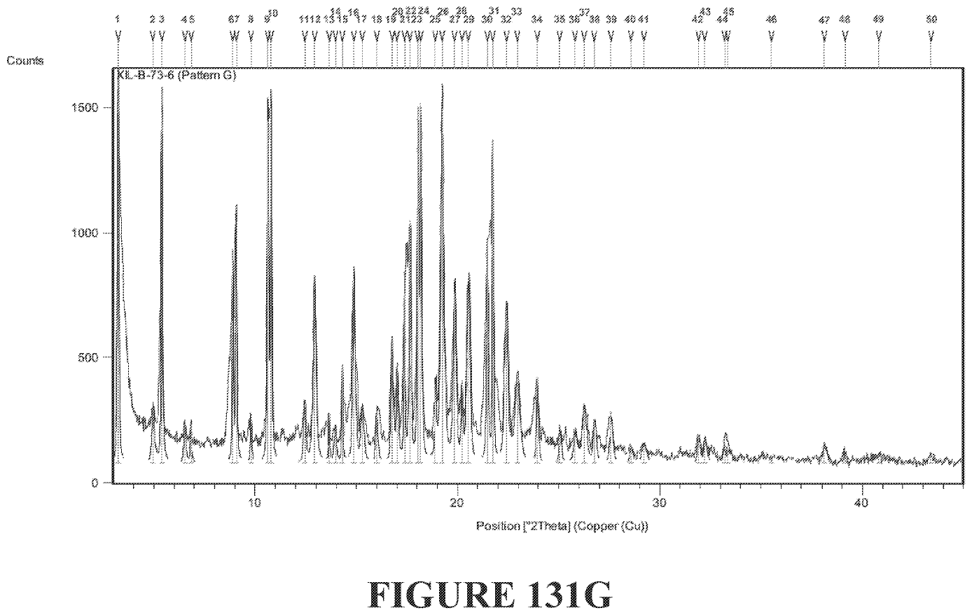

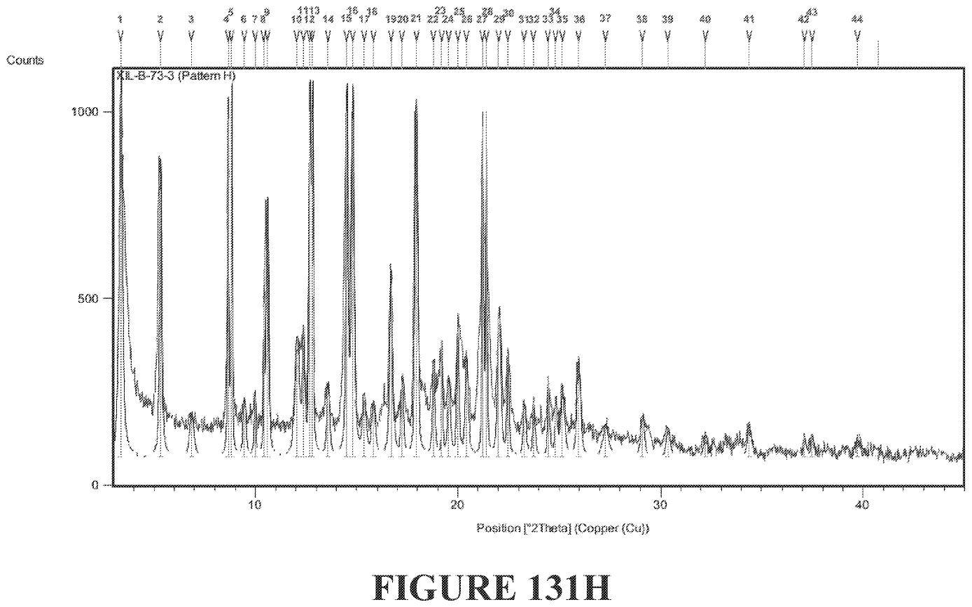

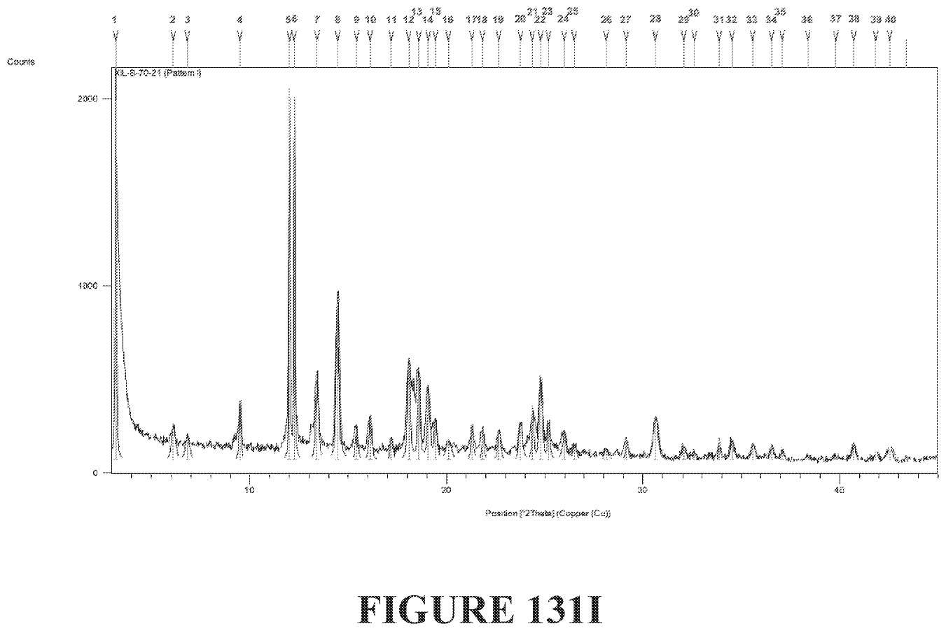

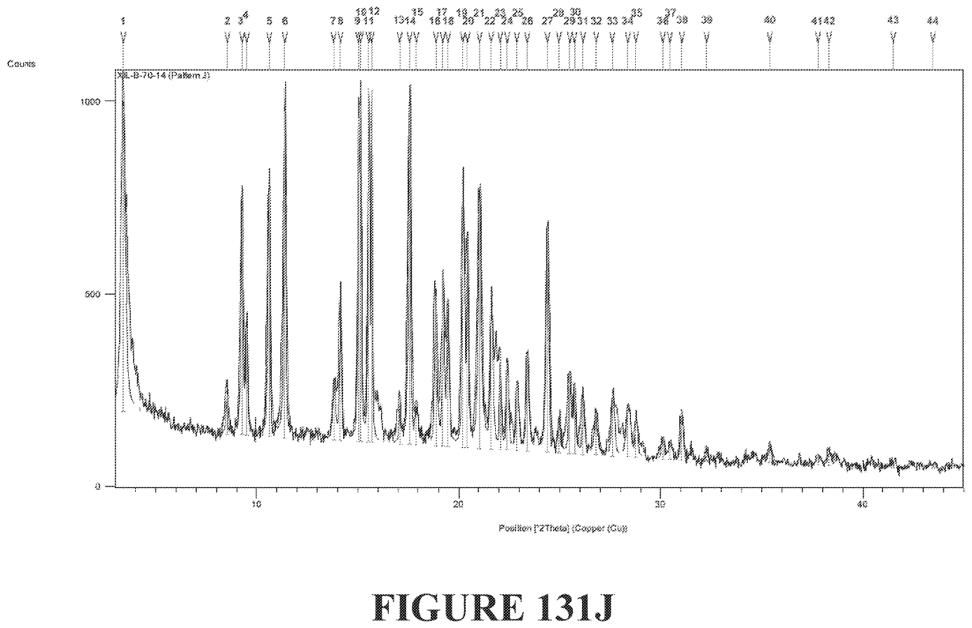

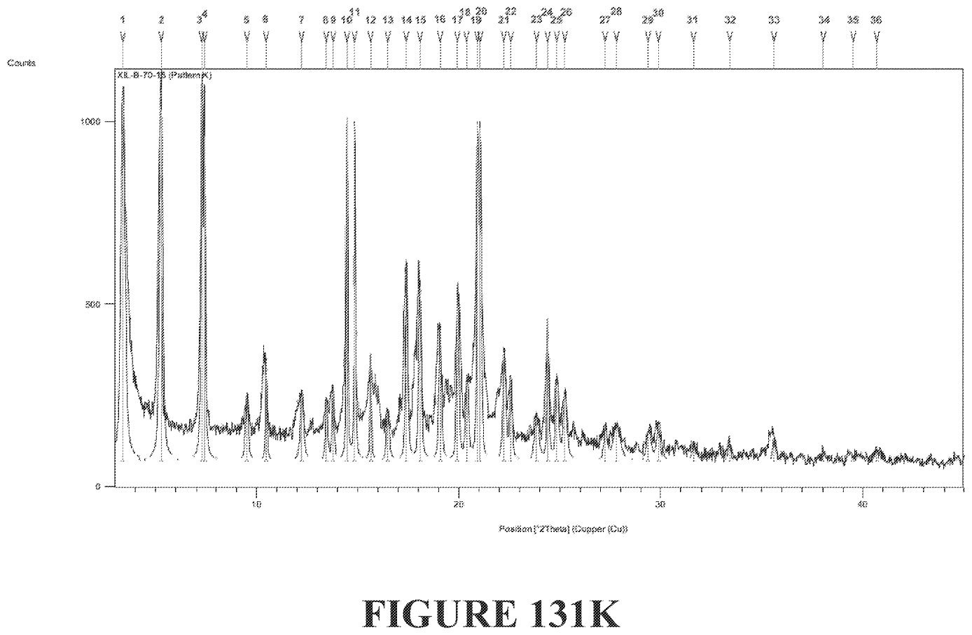

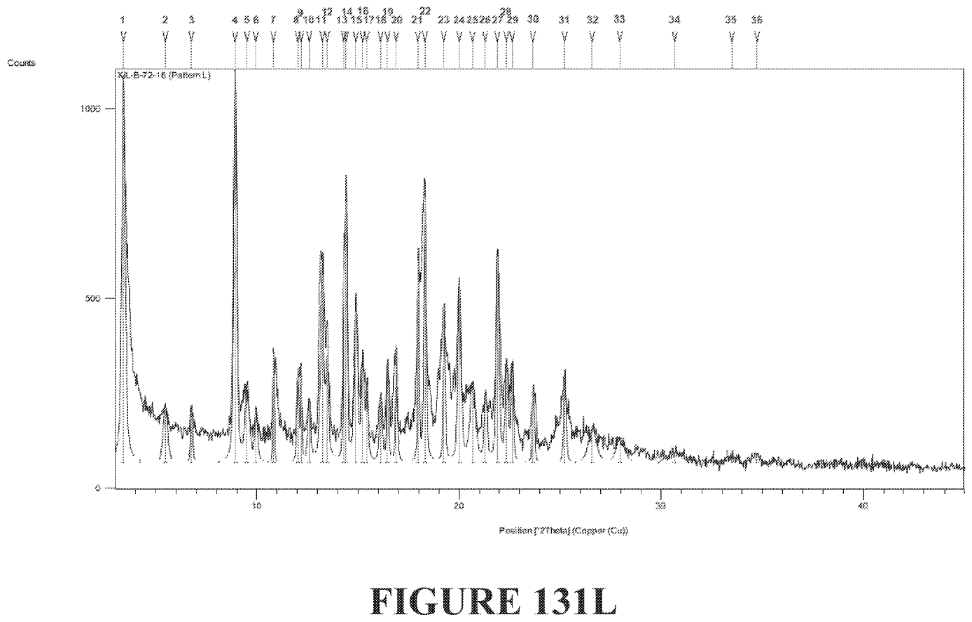

In certain aspects, the present disclosure provides Form A of Formula (I) (hereinafter “Form A”) as characterized by an X-ray powder diffraction pattern substantially as set forth in Panel A of FIG. 1 or FIG. 131A. In certain aspects, the present disclosure provides Form A of Formula (I) as characterized by an X-ray powder diffraction pattern comprising peaks at 3.3±0.3°2θ, 30.3±0.3°2θ, and 20.0±0.3°2θ. In some cases, relative intensities of the peaks at 3.3±0.3°2θ, 30.3±0.3°2θ, and 20.0±0.3°2θ differ by no more than 20%, by no more than 18%, by no more than 16%, by no more than 14%, by no more than 12%, by no more than 10%, by no more than 8%, by no more than 6%, or by no more than 5% (e.g., as determined by Gaussian or Lorentzian fitting peaks in the X-ray powder diffraction pattern). In some cases, the peaks at 3.3±0.3°2θ, 30.3±0.3°2θ, and 20.0±0.3°2θ each have an intensity which is at least 1.3-times, at least 1.4-times, at least 1.5-times, at least 1.6-times, at least 1.7-times, at least 1.8-times, or at least 1.9-times that of the 4^{th} most intense peak in the X-ray powder diffraction pattern. In some cases, the X-ray powder diffraction pattern further comprises at least one, at least two, or at least three peaks selected from 12.1±0.3°2θ, 15.6±0.3°2θ, and 6.0±0.3°2θ. In some cases, the X-ray powder diffraction pattern further comprises at least one, at least two, or at least three peaks selected from 21.7±0.3°2θ, 10.6±0.3°2θ, and 21.6±0.3°2θ. In some cases, Formula (I) is at least 50%, at least 60%, at least 70%, at least 80%, at least 90%, at least 95%, at least 98%, at least 99%, at least 99.5%, or at least 99.9% Form A by weight.

Form A of Formula (I) can exhibit an X-ray powder diffraction pattern shown in FIG. 131A with peaks 1-28 corresponding to the peak numbers in TABLE 27. Peaks in FIG. 131A (numbered from 1 to 28 in order from left to right) and TABLE 27 are provided as relative intensities (Rel. Int. %) standardized against peak 1 (the peak with the highest intensity).

TABLE 27

| | Pos. | Height | FWHM | d-spacing | Rel. Int. |
|---|---|---|---|---|---|
| Peak | [°2θ] | [cts] | [°2θ] | [Å] | [%] |
| 1 | 3.2960 | 1112.27 | 0.2362 | 26.80703 | 100.00 |
| 2 | 6.0199 | 378.59 | 0.1378 | 14.68188 | 34.04 |
| 3 | 8.3128 | 119.91 | 0.1181 | 10.63665 | 10.78 |
| 4 | 9.8896 | 138.87 | 0.1968 | 8.94396 | 12.49 |
| 5 | 10.6459 | 275.83 | 0.1574 | 8.31022 | 24.80 |
| 6 | 12.0521 | 523.07 | 0.1574 | 7.34359 | 47.03 |
| 7 | 14.0013 | 58.94 | 0.4723 | 6.32536 | 5.30 |
| 8 | 15.1652 | 217.93 | 0.1378 | 5.84242 | 19.59 |
| 9 | 15.6284 | 404.36 | 0.1181 | 5.67028 | 36.35 |
| 10 | 16.1456 | 113.19 | 0.1378 | 5.48980 | 10.18 |
| 11 | 16.5103 | 94.51 | 0.1181 | 5.36934 | 8.50 |
| 12 | 17.3476 | 60.40 | 0.1968 | 5.11202 | 5.43 |
| 13 | 18.0863 | 193.99 | 0.1574 | 4.90487 | 17.44 |
| 14 | 20.0219 | 1078.06 | 0.1968 | 4.43485 | 96.92 |
| 15 | 20.5124 | 219.42 | 0.1574 | 4.32989 | 19.73 |
| 16 | 21.1101 | 148.86 | 0.1968 | 4.20862 | 13.38 |
| 17 | 21.7452 | 349.17 | 0.1378 | 4.08713 | 31.39 |
| 18 | 22.3668 | 75.63 | 0.1968 | 3.97492 | 6.80 |
| 19 | 23.1101 | 79.91 | 0.1378 | 3.84873 | 7.18 |
| 20 | 24.2155 | 105.65 | 0.1574 | 3.67549 | 9.50 |
| 21 | 25.0441 | 71.95 | 0.3936 | 3.55573 | 6.47 |
| 22 | 26.1207 | 236.36 | 0.2362 | 3.41157 | 21.25 |
| 23 | 30.3494 | 1080.85 | 0.2952 | 2.94517 | 97.18 |
| 24 | 31.1844 | 174.32 | 0.1181 | 2.86819 | 15.67 |
| 25 | 32.2120 | 53.67 | 0.2362 | 2.77900 | 4.82 |
| 26 | 35.8837 | 50.21 | 0.2362 | 2.50263 | 4.51 |
| 27 | 36.6848 | 209.45 | 0.2362 | 2.44979 | 18.83 |
| 28 | 37.3158 | 122.65 | 0.2362 | 2.40980 | 11.03 |

In certain aspects, the present disclosure provides Form B of Formula (I) (hereinafter “Form B”) as characterized by an X-ray powder diffraction pattern substantially as set forth in Panel B of FIG. 1 or FIG. 131B. In certain aspects, the present disclosure provides Form B of Formula (I) as characterized by an X-ray powder diffraction pattern comprising peaks at 12.2±0.3°2θ, 3.5±0.3°2θ, and 17.2±0.3°2θ. In some cases, relative intensities of the peaks at 12.2±0.3°2θ and 3.5±0.3°2θ are within at least 10%, at least 9%, at least 8%, at least 7%, at least 6%, at least 5%, at least 4%, or at least 3%. In some cases, the peak at 12.2±0.3°2θ has a 4% to 30% greater relative intensity, a 5% to 25% greater relative intensity, a 6% to 15% greater relative intensity, or a 7.5% to 12.5% greater relative intensity than the peak at 17.2±0.3°2θ. In some cases, the X-ray powder diffraction pattern further comprises at least one, at least two, or at least three peaks selected from 19.6±0.3°2θ, 13.1±0.3θ2°, and 18.0±0.3020. In some cases, the X-ray powder diffraction pattern further comprises at least one, at least two, at least three, or at least four peaks selected from 20.2±0.3°2θ, 14.1±0.3°2θ, 17.6±0.3°2θ, and 14.8±0.3°2θ. In some cases, Formula (I) is at least 50%, at least 60%, at least 70%, at least 80%, at least 90%, at least 95%, at least 98%, at least 99%, at least 99.5%, or at least 99.9% Form B by weight.

Form B of Formula (I) can exhibit an X-ray powder diffraction pattern shown in FIG. 131B with peaks 1-29 corresponding to the peak numbers in TABLE 28. Peaks in FIG. 131B (numbered from 1 to 29 in order from left to right) and TABLE 28 are provided as relative intensities (Rel. Int. %) standardized against peak 5 (the peak with the highest intensity).

TABLE 28

| | Pos. | Height | FWHM | d-spacing | Rel. Int. |
|---|---|---|---|---|---|
| Peak | [°2θ] | [cts] | [°2θ] | [Å] | [%] |
| 1 | 3.4851 | 605.93 | 0.1181 | 25.35269 | 97.31 |
| 2 | 6.8101 | 53.90 | 0.1574 | 12.97997 | 8.66 |
| 3 | 9.3371 | 166.38 | 0.1181 | 9.47201 | 26.72 |
| 4 | 9.7681 | 198.93 | 0.1378 | 9.05494 | 31.95 |
| 5 | 12.1664 | 622.67 | 0.1574 | 7.27487 | 100.00 |
| 6 | 13.0956 | 296.64 | 0.1968 | 6.76072 | 47.64 |
| 7 | 14.1365 | 269.10 | 0.1378 | 6.26517 | 43.22 |
| 8 | 14.8071 | 251.24 | 0.0984 | 5.98288 | 40.35 |
| 9 | 15.7288 | 197.33 | 0.1378 | 5.63431 | 31.69 |
| 10 | 16.3415 | 136.92 | 0.1181 | 5.42441 | 21.99 |
| 11 | 16.7425 | 159.98 | 0.1574 | 5.29540 | 25.69 |
| 12 | 17.1841 | 564.16 | 0.1378 | 5.16028 | 90.60 |
| 13 | 17.5627 | 252.06 | 0.1574 | 5.04990 | 40.48 |
| 14 | 17.9782 | 274.78 | 0.1574 | 4.93410 | 44.13 |
| 15 | 18.4950 | 222.30 | 0.0787 | 4.79738 | 35.70 |
| 16 | 19.5680 | 461.69 | 0.1378 | 4.53668 | 74.15 |
| 17 | 20.2221 | 272.75 | 0.1574 | 4.39138 | 43.80 |
| 18 | 20.5559 | 160.92 | 0.1181 | 4.32083 | 25.84 |
| 19 | 21.2512 | 91.45 | 0.1968 | 4.18101 | 14.69 |
| 20 | 22.0990 | 216.38 | 0.3936 | 4.02248 | 34.75 |
| 21 | 23.8123 | 116.42 | 0.1181 | 3.73680 | 18.70 |
| 22 | 24.5437 | 143.37 | 0.2362 | 3.62708 | 23.03 |
| 23 | 26.3464 | 67.73 | 0.3149 | 3.38286 | 10.88 |
| 24 | 27.4287 | 42.29 | 0.2362 | 3.25178 | 6.79 |
| 25 | 28.5165 | 46.24 | 0.1968 | 3.13017 | 7.43 |
| 26 | 29.8672 | 53.61 | 0.1574 | 2.99161 | 8.61 |
| 27 | 31.6276 | 17.64 | 0.2362 | 2.82900 | 2.83 |
| 28 | 35.3772 | 8.07 | 0.9446 | 2.53729 | 1.30 |
| 29 | 37.5993 | 6.47 | 0.9446 | 2.39228 | 1.04 |

In certain aspects, the present disclosure provides Form C of Formula (I) (hereinafter “Form C”) as characterized by an X-ray powder diffraction pattern substantially as set forth in Panel C of FIG. 1 or FIG. 131C. In certain aspects, the present disclosure provides Form C of Formula (I) as characterized by an X-ray powder diffraction pattern comprising peaks at 16.3±0.3°2θ, 16.1±0.3°2θ, and 3.2±0.3°2θ. In some cases, relative intensities of the peaks at 16.3±0.3°2θ, 16.1±0.3°2θ, and 3.2±0.3°2θ are within at least 10%, at least 9%, at least 8%, at least 7%, at least 6%, at least 5%, at least 4%, or at least 3%. In some cases, the peaks at 16.3±0.3°2θ, 16.1±0.3°2θ, and 3.2±0.3°2θ are each at least 20%, at least 25%, at least 30%, at least 35%, at least 40%, at least 45%, or at least 50% more intense than the next-most intense peak in the X-ray powder diffraction pattern. In some cases, the X-ray powder diffraction pattern further comprises at least one, at least two, or at least three peaks selected from 21.6±0.3°2θ, 23.2±0.3°2θ, and 21.7±0.3°2θ. In some cases, the X-ray powder diffraction pattern further comprises at least one, at least two, or at least three peaks selected from 16.5±0.3°2θ, 21.4±0.3°2θ, and 10.7±0.3°2θ. In some cases, Formula (I) is at least 50%, at least 60%, at least 70%, at least 80%, at least 90%, at least 95%, at least 98%, at least 99%, at least 99.5%, or at least 99.9% Form C by weight.

Form C of Formula (I) can exhibit an X-ray powder diffraction pattern shown in FIG. 131C with peaks 1-35 corresponding to the peak numbers in TABLE 29. Peaks in FIG. 131C (numbered from 1 to 35 in order from left to right) and TABLE 29 are provided as relative intensities (Rel. Int. %) standardized against peak 12 (the peak with the highest intensity).

TABLE 29

| | Pos. | Height | FWHM | d-spacing | Rel. Int. |
|---|---|---|---|---|---|
| Peak | [°2θ] | [cts] | [°2θ] | [Å] | [%] |
| 1 | 3.1788 | 1853.54 | 0.0787 | 27.79490 | 98.41 |
| 2 | 5.4343 | 219.56 | 0.0787 | 16.26267 | 11.66 |
| 3 | 8.9595 | 106.98 | 0.0787 | 9.87028 | 5.68 |
| 4 | 10.2884 | 65.04 | 0.1574 | 8.59823 | 3.45 |
| 5 | 10.7433 | 465.08 | 0.0590 | 8.23510 | 24.69 |
| 6 | 10.8562 | 450.50 | 0.0590 | 8.14973 | 23.92 |
| 7 | 13.7288 | 145.78 | 0.1968 | 6.45029 | 7.74 |
| 8 | 13.9570 | 122.28 | 0.1181 | 6.34535 | 6.49 |
| 9 | 14.7155 | 133.54 | 0.1378 | 6.01992 | 7.09 |
| 10 | 15.1298 | 82.58 | 0.1181 | 5.85599 | 4.38 |
| 11 | 16.1243 | 1875.68 | 0.0787 | 5.49697 | 99.58 |
| 12 | 16.3122 | 1883.55 | 0.0590 | 5.43407 | 100.00 |
| 13 | 16.4898 | 919.20 | 0.0984 | 5.37597 | 48.80 |
| 14 | 17.5110 | 354.35 | 0.1181 | 5.06467 | 18.81 |
| 15 | 17.7795 | 154.11 | 0.1181 | 4.98880 | 8.18 |
| 16 | 19.7250 | 188.43 | 0.0984 | 4.50091 | 10.00 |
| 17 | 21.3731 | 679.90 | 0.1378 | 4.15743 | 36.10 |
| 18 | 21.6370 | 1169.74 | 0.1200 | 4.10392 | 62.10 |
| 19 | 21.7402 | 923.33 | 0.0960 | 4.09482 | 49.02 |
| 20 | 22.2486 | 158.22 | 0.2400 | 3.99246 | 8.40 |
| 21 | 23.1842 | 984.14 | 0.1440 | 3.83342 | 52.25 |
| 22 | 24.5657 | 35.02 | 0.4800 | 3.62088 | 1.86 |
| 23 | 25.4046 | 52.15 | 0.2880 | 3.50318 | 2.77 |
| 24 | 27.1738 | 77.20 | 0.1920 | 3.27899 | 4.10 |
| 25 | 27.8108 | 55.04 | 0.3360 | 3.20531 | 2.92 |
| 26 | 29.4312 | 84.34 | 0.1920 | 3.03242 | 4.48 |
| 27 | 31.2947 | 54.90 | 0.2400 | 2.85597 | 2.91 |
| 28 | 32.4993 | 45.46 | 0.1920 | 2.75281 | 2.41 |
| 29 | 32.8453 | 113.30 | 0.0720 | 2.72459 | 6.02 |
| 30 | 33.6986 | 24.61 | 0.2880 | 2.65753 | 1.31 |
| 31 | 34.9925 | 31.66 | 0.1440 | 2.56217 | 1.68 |
| 32 | 38.4530 | 128.82 | 0.2400 | 2.33918 | 6.84 |
| 33 | 39.2205 | 26.43 | 0.2400 | 2.29515 | 1.40 |
| 34 | 39.7919 | 99.24 | 0.0960 | 2.26350 | 5.27 |
| 35 | 42.7625 | 17.04 | 0.4800 | 2.11289 | 0.90 |

In certain aspects, the present disclosure provides Form D of Formula (I) (hereinafter “Form D”) as characterized by an X-ray powder diffraction pattern substantially as set forth in Panel D of FIG. 1 or FIG. 131D. In certain aspects, the present disclosure provides Form D of Formula (I) as characterized by an X-ray powder diffraction pattern comprising peaks at 20.1±0.3°2θ, 18.3±0.3°2θ, and 18.1±0.3°2θ. In some cases, relative intensities of the peaks at 20.1±0.3°2°, 18.3±0.3°2θ, and 18.1±0.3°2θ are within at least 86, at least 70, at least 6%, at least 5%, at least 4%, at least 3%, at least 2%, at least 1.5%, or at least 1%. In some cases, the X-ray powder diffraction pattern further comprises at least one or at least two peaks selected from 20.3±0.3°2° and 17.1±0.3°2θ. In some cases, the X-ray powder diffraction pattern further comprises at least one, at least two, at least three, or at least four peaks selected from 3.4±0.3°2θ, 19.6±0.3°2θ, 23.4±0.3°2θ, and 25.1±0.3°2θ. In some cases, Formula (I) is at least 50%, at least 60%, at least 70%, at least 80%, at least 90%, at least 95%, at least 98%, at least 99%, at least 99.5%, or at least 99.9% Form D by weight.

Form D of Formula (I) can exhibit an X-ray powder diffraction pattern shown in FIG. 131D with peaks 1-45 corresponding to the peak numbers in TABLE 30. Peaks in FIG. 131D (numbered from 1 to 45 in order from left to right) and TABLE 30 are provided as relative intensities (Rel. Int. %) standardized against peak 14 (the peak with the highest intensity).

TABLE 30

| | Pos. | Height | FWHM | d-spacing | Rel. Int. |
|---|---|---|---|---|---|
| Peak | [°2θ] | [cts] | [°2θ] | [Å] | [%] |
| 1 | 3.3807 | 804.97 | 0.1574 | 26.13552 | 86.88 |
| 2 | 10.7019 | 209.78 | 0.1378 | 8.26686 | 22.64 |
| 3 | 11.2930 | 137.79 | 0.0590 | 7.83548 | 14.87 |
| 4 | 12.2671 | 59.93 | 0.2362 | 7.21537 | 6.47 |
| 5 | 12.8539 | 144.43 | 0.0984 | 6.88730 | 15.59 |
| 6 | 13.7518 | 225.24 | 0.0787 | 6.43955 | 24.31 |
| 7 | 15.1883 | 135.61 | 0.1378 | 5.83358 | 14.64 |
| 8 | 15.7605 | 84.44 | 0.1181 | 5.62304 | 9.11 |
| 9 | 16.2447 | 195.95 | 0.0984 | 5.45652 | 21.15 |
| 10 | 17.1275 | 889.06 | 0.1968 | 5.17721 | 95.95 |
| 11 | 18.0987 | 919.49 | 0.0590 | 4.90154 | 99.24 |
| 12 | 18.3233 | 920.65 | 0.0590 | 4.84194 | 99.36 |
| 13 | 19.5939 | 564.91 | 0.1181 | 4.53074 | 60.97 |
| 14 | 20.0970 | 926.55 | 0.0590 | 4.41844 | 100.00 |
| 15 | 20.3167 | 893.63 | 0.0787 | 4.37116 | 96.45 |
| 16 | 20.7559 | 328.31 | 0.1181 | 4.27965 | 35.43 |
| 17 | 21.3731 | 206.40 | 0.0787 | 4.15744 | 22.28 |
| 18 | 21.5821 | 338.75 | 0.0984 | 4.11763 | 36.56 |
| 19 | 23.0443 | 127.16 | 0.1378 | 3.85957 | 13.72 |
| 20 | 23.2685 | 307.50 | 0.1200 | 3.81973 | 33.19 |
| 21 | 23.3711 | 367.15 | 0.0720 | 3.81264 | 39.63 |
| 22 | 25.1041 | 346.48 | 0.1200 | 3.54443 | 37.39 |
| 23 | 25.4503 | 263.61 | 0.1440 | 3.49699 | 28.45 |
| 24 | 25.9490 | 95.40 | 0.0720 | 3.43092 | 10.30 |
| 25 | 26.4922 | 60.09 | 0.1200 | 3.36179 | 6.48 |
| 26 | 26.8547 | 28.11 | 0.1440 | 3.31722 | 3.03 |
| 27 | 27.2924 | 219.11 | 0.1680 | 3.26501 | 23.65 |
| 28 | 27.8179 | 70.80 | 0.0720 | 3.20451 | 7.64 |
| 29 | 28.2841 | 236.05 | 0.0960 | 3.15274 | 25.48 |
| 30 | 29.1156 | 185.38 | 0.0720 | 3.06456 | 20.01 |
| 31 | 29.7412 | 125.15 | 0.1440 | 3.00151 | 13.51 |
| 32 | 30.0419 | 77.88 | 0.1440 | 2.97215 | 8.40 |
| 33 | 30.9237 | 13.71 | 0.2880 | 2.88938 | 1.48 |
| 34 | 31.8953 | 46.02 | 0.1440 | 2.80354 | 4.97 |
| 35 | 32.3596 | 100.47 | 0.1920 | 2.76437 | 10.84 |
| 36 | 32.7101 | 87.05 | 0.1200 | 2.73555 | 9.40 |
| 37 | 34.2843 | 41.88 | 0.1440 | 2.61345 | 4.52 |
| 38 | 35.5060 | 25.63 | 0.3840 | 2.52628 | 2.77 |
| 39 | 36.0807 | 49.53 | 0.1920 | 2.48735 | 5.35 |
| 40 | 36.8798 | 26.01 | 0.2880 | 2.43527 | 2.81 |
| 41 | 37.6492 | 14.52 | 0.1920 | 2.38725 | 1.57 |
| 42 | 37.9929 | 81.76 | 0.1440 | 2.36644 | 8.82 |
| 43 | 38.6569 | 66.23 | 0.0720 | 2.32730 | 7.15 |
| 44 | 41.0920 | 23.85 | 0.5760 | 2.19484 | 2.57 |
| 45 | 42.4017 | 25.69 | 0.1920 | 2.13003 | 2.77 |

In certain aspects, the present disclosure provides Form E of Formula (I) (hereinafter “Form E”) as characterized by an X-ray powder diffraction pattern substantially as set forth in Panel E of FIG. 1 or FIG. 131E. In certain aspects, the present disclosure provides Form E of Formula (I) as characterized by an X-ray powder diffraction pattern comprising peaks at 13.4±0.3°2θ, 11.2±0.3°2θ, and 3.1±0.3°2θ. In some cases, the peaks at 13.4±0.3°2θ, 11.2±0.3°2θ, and 3.1±0.3°2θ have relative intensities within 20%, within 18%, within 16%, within 14%, within 12%, within or 10%. In some cases, the X-ray powder diffraction pattern further comprises at least one, at least two, or at least three peaks selected from 9.0±0.3°2θ, 22.2±0.3°2θ, and 14.3±0.3°2θ. In some cases, the X-ray powder diffraction pattern further comprises at least one, at least two, or at least three peaks selected from 14.9±0.3°2θ, 18.4±0.3°2θ, and 16.8±0.3°2θ. In some cases, Formula (I) is at least 50%, at least 60%, at least 70%, at least 80%, at least 90%, at least 95%, at least 98%, at least 99%, at least 99.5%, or at least 99.9% Form E by weight.

Form E of Formula (I) can exhibit an X-ray powder diffraction pattern shown in FIG. 131E with peaks 1-42 corresponding to the peak numbers in TABLE 31. Peaks in FIG. 131E (numbered from 1 to 42 in order from left to right) and TABLE 31 are provided as relative intensities (Rel. Int. %) standardized against peak 8 (the peak with the highest intensity).

TABLE 31

| | Pos. | Height | FWHM | d-spacing | Rel. Int. |
|---|---|---|---|---|---|
| Peak | [°2θ] | [cts] | [°2θ] | [Å] | [%] |
| 1 | 3.1288 | 2796.52 | 0.0984 | 28.23900 | 93.07 |
| 2 | 5.5896 | 439.31 | 0.0787 | 15.81107 | 14.62 |
| 3 | 6.7076 | 136.83 | 0.1181 | 13.17810 | 4.55 |
| 4 | 8.9864 | 2647.38 | 0.1181 | 9.84083 | 88.10 |
| 5 | 11.1995 | 2840.58 | 0.1574 | 7.90065 | 94.53 |
| 6 | 12.1236 | 346.50 | 0.1574 | 7.30044 | 11.53 |
| 7 | 12.7356 | 313.62 | 0.0590 | 6.95101 | 10.44 |
| 8 | 13.4029 | 3004.90 | 0.1378 | 6.60636 | 100.00 |
| 9 | 14.2791 | 1543.51 | 0.0984 | 6.20292 | 51.37 |
| 10 | 14.8763 | 776.94 | 0.1574 | 5.95521 | 25.86 |
| 11 | 15.4692 | 267.12 | 0.1181 | 5.72827 | 8.89 |
| 12 | 16.4527 | 311.61 | 0.0787 | 5.38800 | 10.37 |
| 13 | 16.8300 | 534.79 | 0.1378 | 5.26804 | 17.80 |
| 14 | 17.3362 | 133.67 | 0.1968 | 5.11536 | 4.45 |
| 15 | 18.3876 | 629.53 | 0.1181 | 4.82517 | 20.95 |
| 16 | 19.4152 | 343.40 | 0.1181 | 4.57204 | 11.43 |
| 17 | 19.7879 | 435.24 | 0.1378 | 4.48676 | 14.48 |
| 18 | 20.7568 | 301.05 | 0.1574 | 4.27945 | 10.02 |
| 19 | 21.2491 | 275.94 | 0.0984 | 4.18141 | 9.18 |
| 20 | 22.1947 | 1678.48 | 0.1378 | 4.00535 | 55.86 |
| 21 | 22.8087 | 511.43 | 0.1378 | 3.89890 | 17.02 |
| 22 | 23.1155 | 280.63 | 0.1378 | 3.84784 | 9.34 |
| 23 | 23.5320 | 138.24 | 0.1181 | 3.78068 | 4.60 |
| 24 | 23.9076 | 276.11 | 0.1378 | 3.72212 | 9.19 |
| 25 | 24.8295 | 121.77 | 0.1181 | 3.58598 | 4.05 |
| 26 | 25.3772 | 343.19 | 0.0590 | 3.50981 | 11.42 |
| 27 | 26.3054 | 111.73 | 0.1181 | 3.38803 | 3.72 |
| 28 | 27.1528 | 297.42 | 0.1378 | 3.28419 | 9.90 |
| 29 | 27.9036 | 76.23 | 0.3149 | 3.19751 | 2.54 |
| 30 | 29.4729 | 81.83 | 0.1181 | 3.03073 | 2.72 |
| 31 | 30.4639 | 71.51 | 0.3149 | 2.93436 | 2.38 |
| 32 | 31.2529 | 60.25 | 0.2362 | 2.86206 | 2.01 |
| 33 | 32.1277 | 77.49 | 0.1574 | 2.78610 | 2.58 |
| 34 | 33.8575 | 102.76 | 0.2362 | 2.64761 | 3.42 |
| 35 | 35.0553 | 74.04 | 0.2755 | 2.55984 | 2.46 |
| 36 | 36.6752 | 80.93 | 0.0984 | 2.45041 | 2.69 |
| 37 | 39.1025 | 42.75 | 0.1574 | 2.30371 | 1.42 |
| 38 | 39.6094 | 44.46 | 0.2362 | 2.27539 | 1.48 |
| 39 | 40.1174 | 26.40 | 0.2362 | 2.24775 | 0.88 |
| 40 | 40.9475 | 39.22 | 0.1574 | 2.20408 | 1.31 |
| 41 | 42.4599 | 51.58 | 0.1968 | 2.12900 | 1.72 |
| 42 | 44.1556 | 19.89 | 0.2880 | 2.04940 | 0.66 |

In certain aspects, the present disclosure provides Form F of Formula (I) (hereinafter “Form F”) as characterized by an X-ray powder diffraction pattern substantially as set forth in Panel F of FIG. 1 or FIG. 131F. In certain aspects, the present disclosure provides Form F of Formula (I) as characterized by an X-ray powder diffraction pattern comprising peaks at 14.6±0.3°2θ, 3.4±0.3°2θ, and 9.7±0.3°2θ. In some cases, the X-ray powder diffraction pattern further comprises at least one, at least two, or at least three peaks selected from 18.1±0.3°2θ, 20.2±0.3°2θ, and 16.7±0.3°2θ. In some cases, the X-ray powder diffraction pattern further comprises at least one, at least two, or at least three peaks selected from 17.6±0.3°2θ, 19.2±0.3°2θ, and 17.3±0.3°2θ. In some cases, Formula (I) is at least 50%, at least 60%, at least 70%, at least 80%, at least 90%, at least 95%, at least 98%, at least 99%, at least 99.5%, or at least 99.9% Form F by weight.

Form F of Formula (I) can exhibit an X-ray powder diffraction pattern shown in FIG. 131F with peaks 1-42 corresponding to the peak numbers in TABLE 32. Peaks in FIG. 131F (numbered from 1 to 42 in order from left to right) and TABLE 32 are provided as relative intensities (Rel. Int. %) standardized against peak 10 (the peak with the highest intensity).

TABLE 32

| | Pos. | Height | FWHM | d-spacing | Rel. Int. |
|---|---|---|---|---|---|
| Peak | [°2θ] | [cts] | [°2θ] | [Å] | [%] |
| 1 | 3.3973 | 878.81 | 0.0984 | 26.00790 | 99.35 |
| 2 | 4.8605 | 373.01 | 0.0984 | 18.18110 | 42.17 |
| 3 | 6.4890 | 107.45 | 0.2362 | 13.62151 | 12.15 |
| 4 | 8.9926 | 237.95 | 0.0787 | 9.83403 | 26.90 |
| 5 | 9.6987 | 831.17 | 0.1574 | 9.11963 | 93.96 |
| 6 | 12.0771 | 75.63 | 0.1574 | 7.32847 | 8.55 |
| 7 | 12.9286 | 104.47 | 0.1181 | 6.84765 | 11.81 |
| 8 | 13.3135 | 102.84 | 0.1181 | 6.65056 | 11.63 |
| 9 | 13.8715 | 90.25 | 0.1574 | 6.38424 | 10.20 |
| 10 | 14.5507 | 884.59 | 0.1771 | 6.08773 | 100.00 |
| 11 | 14.9927 | 288.20 | 0.1378 | 5.90926 | 32.58 |
| 12 | 15.1839 | 252.19 | 0.1181 | 5.83527 | 28.51 |
| 13 | 16.0261 | 99.49 | 0.2362 | 5.53044 | 11.25 |
| 14 | 16.7413 | 527.23 | 0.1574 | 5.29577 | 59.60 |
| 15 | 17.3394 | 474.91 | 0.1771 | 5.11441 | 53.69 |
| 16 | 17.6040 | 525.74 | 0.1181 | 5.03813 | 59.43 |
| 17 | 18.1276 | 765.30 | 0.1574 | 4.89379 | 86.51 |
| 18 | 18.8784 | 180.33 | 0.0984 | 4.70082 | 20.39 |
| 19 | 19.1876 | 525.30 | 0.1968 | 4.62575 | 59.38 |
| 20 | 19.8171 | 287.78 | 0.0590 | 4.48021 | 32.53 |
| 21 | 20.1716 | 558.55 | 0.1574 | 4.40226 | 63.14 |
| 22 | 20.5501 | 289.01 | 0.1378 | 4.32203 | 32.67 |
| 23 | 21.2780 | 144.26 | 0.1181 | 4.17580 | 16.31 |
| 24 | 21.9896 | 134.93 | 0.1181 | 4.04225 | 15.25 |
| 25 | 22.4082 | 98.24 | 0.1181 | 3.96767 | 11.11 |
| 26 | 22.8433 | 150.02 | 0.2362 | 3.89308 | 16.96 |
| 27 | 23.4537 | 130.58 | 0.1181 | 3.79312 | 14.76 |
| 28 | 23.8790 | 255.55 | 0.1968 | 3.72651 | 28.89 |
| 29 | 24.4854 | 104.76 | 0.1181 | 3.63559 | 11.84 |
| 30 | 25.0325 | 58.49 | 0.2755 | 3.55736 | 6.61 |
| 31 | 25.8026 | 162.16 | 0.3149 | 3.45291 | 18.33 |
| 32 | 26.2030 | 122.67 | 0.1181 | 3.40103 | 13.87 |
| 33 | 27.0129 | 47.61 | 0.2755 | 3.30088 | 5.38 |
| 34 | 27.5272 | 114.09 | 0.1968 | 3.24037 | 12.90 |
| 35 | 28.5201 | 25.68 | 0.1968 | 3.12978 | 2.90 |
| 36 | 29.8940 | 20.60 | 0.7872 | 2.98899 | 2.33 |
| 37 | 33.2948 | 16.50 | 0.3149 | 2.69106 | 1.86 |
| 38 | 35.5871 | 15.13 | 0.6298 | 2.52280 | 1.71 |
| 39 | 37.1612 | 25.63 | 0.1968 | 2.41947 | 2.90 |
| 40 | 40.7158 | 28.61 | 0.1968 | 2.21608 | 3.23 |
| 41 | 41.6200 | 16.59 | 0.2362 | 2.17000 | 1.88 |
| 42 | 43.3325 | 31.40 | 0.4800 | 2.08641 | 3.55 |

In certain aspects, the present disclosure provides Form G of Formula (I) (hereinafter “Form G”) as characterized by an X-ray powder diffraction pattern substantially as set forth in Panel G of FIG. 1 or FIG. 131G. In certain aspects, the present disclosure provides Form G of Formula (I) as characterized by an X-ray powder diffraction pattern comprising peaks at 18.2±0.3°2θ, 3.2±0.3°2θ, and 18.0±0.3°2θ. In some cases, the X-ray powder diffraction pattern further comprises at least one, at least two, or at least three peaks selected from 10.8±0.30°2θ, 19.2±0.3°2θ, and 5.4±0.3°2θ. In some cases, the X-ray powder diffraction pattern further comprises at least one or at least two peaks selected from 10.6±0.3°2θ and 21.7±0.3°2θ. In some cases, Formula (I) is at least 50%, at least 60%, at least 70%, at least 80%, at least 90%, at least 95%, at least 98%, at least 99%, at least 99.5%, or at least 99.9% Form G by weight.

Form G of Formula (I) can exhibit an X-ray powder diffraction pattern shown in FIG. 131G with peaks 1-50 corresponding to the peak numbers in TABLE 33. Peaks in FIG. 131G (numbered from 1 to 50 in order from left to right) and TABLE 33 are provided as relative intensities (Rel. Int. %) standardized against peak 24 (the peak with the highest intensity).

TABLE 33

| | Pos. | Height | FWHM | d-spacing | Rel. Int. |
|---|---|---|---|---|---|
| Peak | [°2θ] | [cts] | [°2θ] | [Å] | [%] |
| 1 | 3.2234 | 1429.90 | 0.0787 | 27.41028 | 99.33 |
| 2 | 4.9541 | 213.17 | 0.1574 | 17.83787 | 14.81 |
| 3 | 5.3931 | 1402.31 | 0.0787 | 16.38689 | 97.41 |
| 4 | 6.5175 | 157.05 | 0.1181 | 13.56210 | 10.91 |
| 5 | 6.8214 | 154.58 | 0.0787 | 12.95858 | 10.74 |
| 6 | 8.8834 | 811.50 | 0.0590 | 9.95470 | 56.37 |
| 7 | 9.0614 | 967.14 | 0.0787 | 9.75957 | 67.18 |
| 8 | 9.7911 | 188.54 | 0.0787 | 9.03378 | 13.10 |
| 9 | 10.6238 | 1367.62 | 0.0787 | 8.32748 | 95.00 |
| 10 | 10.7894 | 1424.44 | 0.0787 | 8.20006 | 98.95 |
| 11 | 12.4382 | 223.76 | 0.1181 | 7.11650 | 15.54 |
| 12 | 12.9237 | 683.91 | 0.1771 | 6.85022 | 47.51 |
| 13 | 13.6467 | 205.03 | 0.0590 | 6.48888 | 14.24 |
| 14 | 13.9493 | 140.87 | 0.1378 | 6.34881 | 9.79 |
| 15 | 14.3028 | 393.43 | 0.0590 | 6.19268 | 27.33 |
| 16 | 14.8742 | 737.53 | 0.1378 | 5.95604 | 51.23 |
| 17 | 15.2863 | 208.61 | 0.1574 | 5.79638 | 14.49 |
| 18 | 15.9972 | 189.45 | 0.1181 | 5.54037 | 13.16 |
| 19 | 16.7623 | 481.68 | 0.1181 | 5.28916 | 33.46 |
| 20 | 17.0146 | 371.17 | 0.1181 | 5.21130 | 25.78 |
| 21 | 17.3642 | 657.21 | 0.0787 | 5.10718 | 45.65 |
| 22 | 17.6452 | 970.26 | 0.0984 | 5.02648 | 67.40 |
| 23 | 18.0385 | 1426.72 | 0.0787 | 4.91775 | 99.10 |
| 24 | 18.1866 | 1439.61 | 0.0787 | 4.87803 | 100.00 |
| 25 | 18.9021 | 317.57 | 0.0787 | 4.69496 | 22.06 |
| 26 | 19.2341 | 1413.17 | 0.1771 | 4.61467 | 98.16 |
| 27 | 19.8519 | 679.92 | 0.1574 | 4.47244 | 47.23 |
| 28 | 20.2066 | 316.08 | 0.0787 | 4.39472 | 21.96 |
| 29 | 20.5197 | 680.06 | 0.1771 | 4.32838 | 47.24 |
| 30 | 21.4623 | 877.22 | 0.0984 | 4.14036 | 60.93 |
| 31 | 21.7347 | 1287.40 | 0.0590 | 4.08907 | 89.43 |
| 32 | 22.4105 | 593.04 | 0.2165 | 3.96728 | 41.19 |
| 33 | 22.9502 | 325.14 | 0.2755 | 3.87518 | 22.59 |
| 34 | 23.9309 | 343.60 | 0.0984 | 3.71856 | 23.87 |
| 35 | 25.0605 | 124.40 | 0.1181 | 3.55344 | 8.64 |
| 36 | 25.8162 | 112.48 | 0.1968 | 3.45111 | 7.81 |
| 37 | 26.2467 | 214.28 | 0.2362 | 3.39547 | 14.88 |
| 38 | 26.7658 | 165.90 | 0.1574 | 3.33079 | 11.52 |
| 39 | 27.5891 | 184.07 | 0.1574 | 3.23324 | 12.79 |
| 40 | 28.5641 | 67.72 | 0.1378 | 3.12505 | 4.70 |
| 41 | 29.2043 | 78.35 | 0.2362 | 3.05799 | 5.44 |
| 42 | 31.8969 | 115.17 | 0.1181 | 2.80573 | 8.00 |
| 43 | 32.2256 | 86.60 | 0.1574 | 2.77786 | 6.02 |
| 44 | 33.2055 | 70.88 | 0.1440 | 2.69585 | 4.92 |
| 45 | 33.3418 | 50.59 | 0.3149 | 2.68737 | 3.51 |
| 46 | 35.5185 | 23.77 | 0.9446 | 2.52752 | 1.65 |
| 47 | 38.1374 | 66.63 | 0.2362 | 2.35976 | 4.63 |
| 48 | 39.1479 | 36.95 | 0.1968 | 2.30115 | 2.57 |
| 49 | 40.8244 | 29.76 | 0.7872 | 2.21043 | 2.07 |
| 50 | 43.3927 | 29.23 | 0.2880 | 2.08365 | 2.03 |

In certain aspects, the present disclosure provides Form H of Form ula (I) (hereinafter “Form H”) as characterized by an X-ray powder diffraction pattern substantially as set forth in Panel Hof FIG. 1 or FIG. 131H. In certain aspects, the present disclosure provides Form H of Formula (I) as characterized by an X-ray powder diffraction pattern comprising peaks at 8.8±0.3°2θ, 3.4±0.3°2θ, and 21.4±0.3°2θ. In some cases, the X-ray powder diffraction pattern further comprises at least one, at least two, at least three, or at least four peaks selected from 17.9±0.3°2θ, 14.5±0.3°2θ, 12.7±0.3°2θ, and 8.7±0.3°2θ. In some cases, the X-ray powder diffraction pattern further comprises at least one, at least two, or at least three peaks selected from 14.8±0.3°2θ, 12.8±0.3°2θ, and 21.2±0.3°2θ. In some cases, Formula (I) is at least 50%, at least 60%, at least 70%, at least 80%, at least 90%, at least 95%, at least 98%, at least 99%, at least 99.5%, or at least 99.9% Form H by weight.

Form H of Formula (I) can exhibit an X-ray powder diffraction pattern shown in FIG. 131H with peaks 1-45 corresponding to the peak numbers in TABLE 34. Peaks in FIG. 131H (numbered from 1 to 45 in order from left to right) and TABLE 34 are provided as relative intensities (Rel. Int. %) standardized against peak 5 (the peak with the highest intensity).

TABLE 34

| | Pos. | Height | FWHM | d-spacing | Rel. Int. |
|---|---|---|---|---|---|
| Peak | [°2θ] | [cts] | [°2θ] | [Å] | [%] |
| 1 | 3.3567 | 935.17 | 0.1968 | 26.32183 | 98.03 |
| 2 | 5.3274 | 715.56 | 0.1378 | 16.58874 | 75.01 |
| 3 | 6.8519 | 107.25 | 0.2362 | 12.90089 | 11.24 |
| 4 | 8.6570 | 902.51 | 0.0787 | 10.21448 | 94.60 |
| 5 | 8.8227 | 953.99 | 0.0590 | 10.02309 | 100.00 |
| 6 | 9.4532 | 142.40 | 0.1574 | 9.35591 | 14.93 |
| 7 | 9.9671 | 160.09 | 0.0984 | 8.87459 | 16.78 |
| 8 | 10.4143 | 413.13 | 0.0590 | 8.49456 | 43.31 |
| 9 | 10.5908 | 637.59 | 0.1378 | 8.35339 | 66.83 |
| 10 | 12.0498 | 281.93 | 0.2755 | 7.34499 | 29.55 |
| 11 | 12.3716 | 294.06 | 0.1181 | 7.15466 | 30.82 |
| 12 | 12.6897 | 922.02 | 0.0984 | 6.97603 | 96.65 |
| 13 | 12.8088 | 895.65 | 0.0787 | 6.91141 | 93.88 |
| 14 | 13.5925 | 184.37 | 0.1574 | 6.51464 | 19.33 |
| 15 | 14.5132 | 922.50 | 0.1181 | 6.10337 | 96.70 |
| 16 | 14.7978 | 900.81 | 0.1574 | 5.98662 | 94.43 |
| 17 | 15.3641 | 145.44 | 0.1968 | 5.76722 | 15.25 |
| 18 | 15.8129 | 134.98 | 0.1968 | 5.60453 | 14.15 |
| 19 | 16.6940 | 462.40 | 0.1771 | 5.31067 | 48.47 |
| 20 | 17.2338 | 204.11 | 0.1181 | 5.14553 | 21.40 |
| 21 | 17.9323 | 923.15 | 0.1378 | 4.94662 | 96.77 |
| 22 | 18.7899 | 253.11 | 0.1378 | 4.72275 | 26.53 |
| 23 | 19.2075 | 310.35 | 0.0590 | 4.62101 | 32.53 |
| 24 | 19.5443 | 198.59 | 0.1574 | 4.54212 | 20.82 |
| 25 | 19.9858 | 335.84 | 0.1181 | 4.44276 | 35.20 |
| 26 | 20.4186 | 252.04 | 0.1574 | 4.34957 | 26.42 |
| 27 | 21.2172 | 880.60 | 0.0787 | 4.18763 | 92.31 |
| 28 | 21.3885 | 926.84 | 0.0590 | 4.15448 | 97.15 |
| 29 | 22.0230 | 381.99 | 0.1771 | 4.03619 | 40.04 |
| 30 | 22.4511 | 272.78 | 0.0984 | 3.96020 | 28.59 |
| 31 | 23.2777 | 135.15 | 0.1574 | 3.82140 | 14.17 |
| 32 | 23.7319 | 144.48 | 0.1181 | 3.74928 | 15.14 |
| 33 | 24.4508 | 215.03 | 0.0984 | 3.64065 | 22.54 |
| 34 | 24.8228 | 159.16 | 0.1181 | 3.58692 | 16.68 |
| 35 | 25.1435 | 180.20 | 0.1968 | 3.54190 | 18.89 |
| 36 | 25.9393 | 248.67 | 0.1968 | 3.43502 | 26.07 |
| 37 | 27.2704 | 79.01 | 0.3149 | 3.27030 | 8.28 |
| 38 | 29.0854 | 105.78 | 0.1968 | 3.07022 | 11.09 |
| 39 | 30.3506 | 73.72 | 0.1968 | 2.94506 | 7.73 |
| 40 | 32.2090 | 53.36 | 0.2362 | 2.77925 | 5.59 |
| 41 | 34.3599 | 75.24 | 0.2362 | 2.61004 | 7.89 |
| 42 | 37.1003 | 55.34 | 0.1181 | 2.42330 | 5.80 |
| 43 | 37.4831 | 55.57 | 0.1574 | 2.39943 | 5.82 |
| 44 | 39.7395 | 42.20 | 0.2880 | 2.26637 | 4.42 |
| 45 | 40.7418 | 14.21 | 0.7680 | 2.21839 | 1.49 |

In certain aspects, the present disclosure provides Form I of Formula (I) (hereinafter “Form I”) as characterized by an X-ray powder diffraction pattern substantially as set forth in Panel I of FIG. 1 or FIG. 131I. In certain aspects, the present disclosure provides Form I of Formula (I) as characterized by an X-ray powder diffraction pattern comprising peaks at 12.0±0.3°2θ, 12.3±0.3°2θ, and 3.2±0.3°2θ. In some cases, the peaks at 12.0±0.3°2θ, 12.3±0.3°2θ, and 3.2±0.3°2θ are at least 1.5, at least 1.6, at least 1.7, at least 1.8, at least 1.9, or at least 2-times as intense as the next most intense peak. In some cases, the X-ray powder diffraction pattern further comprises at least one, at least two, or at least three peaks selected from 14.5±0.3°2θ, 18.1±0.3°2θ, and 13.4±0.3°2θ. In some cases, the X-ray powder diffraction pattern further comprises at least one, at least two, or at least three peaks selected from 18.6±0.3°2θ, 24.8±0.32θ, and 19.1±0.3°2θ. In some cases, Formula (I) is at least 50%, at least 60%, at least 70%, at least 80%, at least 90%, at least 95%, at least 98%, at least 99%, at least 99.5%, or at least 99.9% Form I by weight.

Form I of Formula (I) can exhibit an X-ray powder diffraction pattern shown in FIG. 131I with peaks 1-41 corresponding to the peak numbers in TABLE 35. Peaks in FIG. 131I (numbered from 1 to 41 in order from left to right) and TABLE 35 are provided as relative intensities (Rel. Int. %) standardized against peak 5 (the peak with the highest intensity).

TABLE 35

| | Pos. | Height | FWHM | d-spacing | Rel. Int. |
|---|---|---|---|---|---|
| Peak | [°2θ] | [cts] | [°2θ] | [Å] | [%] |
| 1 | 3.1737 | 1927.24 | 0.0787 | 27.83984 | 96.83 |
| 2 | 6.1138 | 173.49 | 0.2362 | 14.45653 | 8.72 |
| 3 | 6.8194 | 121.13 | 0.2362 | 12.96224 | 6.09 |
| 4 | 9.4957 | 298.83 | 0.1181 | 9.31410 | 15.01 |
| 5 | 12.0164 | 1990.34 | 0.0590 | 7.36535 | 100.00 |
| 6 | 12.2552 | 1931.70 | 0.0590 | 7.22234 | 97.05 |
| 7 | 13.4100 | 446.68 | 0.1574 | 6.60289 | 22.44 |
| 8 | 14.4701 | 840.42 | 0.1771 | 6.12145 | 42.23 |
| 9 | 15.4216 | 169.00 | 0.1378 | 5.74585 | 8.49 |
| 10 | 16.1190 | 224.56 | 0.1574 | 5.49877 | 11.28 |
| 11 | 17.1920 | 117.21 | 0.1574 | 5.15792 | 5.89 |
| 12 | 18.0735 | 513.19 | 0.1574 | 4.90829 | 25.78 |
| 13 | 18.5890 | 445.77 | 0.1378 | 4.77333 | 22.40 |
| 14 | 19.0819 | 352.62 | 0.1574 | 4.65112 | 17.72 |
| 15 | 19.4417 | 205.91 | 0.1574 | 4.56585 | 10.35 |
| 16 | 20.0919 | 97.39 | 0.3149 | 4.41955 | 4.89 |
| 17 | 21.2913 | 186.64 | 0.1378 | 4.17322 | 9.38 |
| 18 | 21.8378 | 182.60 | 0.0984 | 4.07000 | 9.17 |
| 19 | 22.6583 | 161.15 | 0.1574 | 3.92445 | 8.10 |
| 20 | 23.7565 | 195.65 | 0.1968 | 3.74545 | 9.83 |
| 21 | 24.3702 | 284.76 | 0.0984 | 3.65251 | 14.31 |
| 22 | 24.7730 | 443.19 | 0.1574 | 3.59402 | 22.27 |
| 23 | 25.1853 | 197.35 | 0.1181 | 3.53611 | 9.92 |
| 24 | 25.9895 | 142.74 | 0.2362 | 3.42850 | 7.17 |
| 25 | 26.5027 | 76.09 | 0.1968 | 3.36325 | 3.82 |
| 26 | 28.1098 | 59.22 | 0.2362 | 3.17452 | 2.98 |
| 27 | 29.1500 | 111.70 | 0.1378 | 3.06357 | 5.61 |
| 28 | 30.6055 | 215.51 | 0.2755 | 2.92111 | 10.83 |
| 29 | 32.0622 | 68.43 | 0.2362 | 2.79164 | 3.44 |
| 30 | 32.6105 | 38.29 | 0.1574 | 2.74595 | 1.92 |
| 31 | 33.8600 | 112.13 | 0.0787 | 2.64742 | 5.63 |
| 32 | 34.5260 | 101.13 | 0.2362 | 2.59786 | 5.08 |
| 33 | 35.5730 | 94.54 | 0.1968 | 2.52377 | 4.75 |
| 34 | 36.5358 | 76.30 | 0.1968 | 2.45944 | 3.83 |
| 35 | 37.0694 | 63.72 | 0.1378 | 2.42525 | 3.20 |
| 36 | 38.3776 | 22.90 | 0.2362 | 2.34554 | 1.15 |
| 37 | 39.8099 | 22.43 | 0.3149 | 2.26439 | 1.13 |
| 38 | 40.7033 | 86.86 | 0.1968 | 2.21673 | 4.36 |
| 39 | 41.8346 | 45.04 | 0.1574 | 2.15937 | 2.26 |
| 40 | 42.5288 | 53.00 | 0.3840 | 2.12395 | 2.66 |
| 41 | 43.3778 | 10.57 | 0.5760 | 2.08951 | 0.53 |

In certain aspects, the present disclosure provides Form J of Formula (I) (hereinafter “Form J”) as characterized by an X-ray powder diffraction pattern substantially as set forth in Panel J of FIG. 1 or FIG. 131J. In certain aspects, the present disclosure provides Form J of Formula (I) as characterized by an X-ray powder diffraction pattern comprising peaks at 15.5±0.3°2θ, 15.7±0.3°2θ, and 17.6±0.3°2θ. In some cases, the X-ray powder diffraction pattern further comprises at least one, at least two, or at least three peaks selected from 15.1±0.3°2θ, 11.4±0.3°2θ, and 15.0±0.3°2θ. In some cases, the X-ray powder diffraction pattern further comprises at least one, at least two, or at least three peaks selected from 3.4±0.3°2θ, 20.2±0.3°2θ, and 21.0±0.3°2θ. In some cases, Formula (I) is at least 50%, at least 60%, at least 70%, at least 80%, at least 90%, at least 95%, at least 98%, at least 99%, at least 99.5%, or at least 99.9% Form J by weight.

Form J of Formula (I) can exhibit an X-ray powder diffraction pattern shown in FIG. 131J with peaks 1-44 corresponding to the peak numbers in TABLE 36. Peaks in FIG. 131J (numbered from 1 to 44 in order from left to right) and TABLE 36 are provided as relative intensities (Rel. Int. %) standardized against peak 11 (the peak with the highest intensity).

TABLE 36

| | Pos. | Height | FWHM | d-spacing | Rel. Int. |
|---|---|---|---|---|---|
| Peak | [°2θ] | [cts] | [°2θ] | [Å] | [%] |
| 1 | 3.3863 | 791.71 | 0.2362 | 26.09206 | 86.25 |
| 2 | 8.5444 | 102.20 | 0.1378 | 10.34883 | 11.13 |
| 3 | 9.2805 | 581.68 | 0.1378 | 9.52962 | 63.37 |
| 4 | 9.5152 | 278.89 | 0.0984 | 9.29512 | 30.38 |
| 5 | 10.6154 | 637.09 | 0.1378 | 8.33409 | 69.40 |
| 6 | 11.4035 | 845.27 | 0.1378 | 7.75981 | 92.08 |
| 7 | 13.8154 | 142.61 | 0.1574 | 6.41004 | 15.54 |
| 8 | 14.1490 | 394.31 | 0.1181 | 6.25964 | 42.96 |
| 9 | 15.0410 | 838.77 | 0.0984 | 5.89038 | 91.37 |
| 10 | 15.1461 | 873.26 | 0.0590 | 5.84975 | 95.13 |
| 11 | 15.5378 | 917.95 | 0.0787 | 5.70313 | 100.00 |
| 12 | 15.6841 | 914.58 | 0.0590 | 5.65028 | 99.63 |
| 13 | 17.0854 | 124.57 | 0.0787 | 5.18987 | 13.57 |
| 14 | 17.5725 | 878.76 | 0.1574 | 5.04709 | 95.73 |
| 15 | 17.9134 | 84.69 | 0.1574 | 4.95181 | 9.23 |
| 16 | 18.8620 | 389.66 | 0.1378 | 4.70486 | 42.45 |
| 17 | 19.2256 | 456.61 | 0.0984 | 4.61669 | 49.74 |
| 18 | 19.4582 | 364.42 | 0.1181 | 4.56202 | 39.70 |
| 19 | 20.2122 | 718.42 | 0.1181 | 4.39351 | 78.26 |
| 20 | 20.4335 | 553.19 | 0.0984 | 4.34642 | 60.26 |
| 21 | 21.0457 | 643.43 | 0.1968 | 4.22135 | 70.09 |
| 22 | 21.6131 | 405.90 | 0.1378 | 4.11181 | 44.22 |
| 23 | 22.0495 | 256.15 | 0.0787 | 4.03139 | 27.90 |
| 24 | 22.3951 | 227.65 | 0.1181 | 3.96997 | 24.80 |
| 25 | 22.8860 | 176.15 | 0.1378 | 3.88591 | 19.19 |
| 26 | 23.3984 | 253.80 | 0.1574 | 3.80196 | 27.65 |
| 27 | 24.3869 | 584.97 | 0.1771 | 3.65004 | 63.73 |
| 28 | 24.9448 | 76.97 | 0.1181 | 3.56966 | 8.39 |
| 29 | 25.5039 | 210.05 | 0.1574 | 3.49266 | 22.88 |
| 30 | 25.7376 | 179.16 | 0.0984 | 3.46148 | 19.52 |
| 31 | 26.1643 | 145.48 | 0.1378 | 3.40599 | 15.85 |
| 32 | 26.7953 | 106.50 | 0.2362 | 3.32719 | 11.60 |
| 33 | 27.6118 | 165.38 | 0.1574 | 3.23063 | 18.02 |
| 34 | 28.3802 | 129.79 | 0.2362 | 3.14489 | 14.14 |
| 35 | 28.7777 | 124.21 | 0.0984 | 3.10234 | 13.53 |
| 36 | 30.0966 | 55.80 | 0.1574 | 2.96933 | 6.08 |
| 37 | 30.4570 | 42.42 | 0.1968 | 2.93501 | 4.62 |
| 38 | 31.0488 | 126.16 | 0.1378 | 2.88040 | 13.74 |
| 39 | 32.2707 | 22.23 | 0.1574 | 2.77408 | 2.42 |
| 40 | 35.3991 | 46.77 | 0.1968 | 2.53576 | 5.10 |
| 41 | 37.7828 | 23.95 | 0.2362 | 2.38109 | 2.61 |
| 42 | 38.3198 | 38.95 | 0.1574 | 2.34895 | 4.24 |
| 43 | 41.5116 | 11.79 | 0.3936 | 2.17542 | 1.28 |
| 44 | 43.4674 | 11.17 | 0.4800 | 2.08024 | 1.22 |

In certain aspects, the present disclosure provides Form K of Formula (I) (hereinafter “Form K”) as characterized by an X-ray powder diffraction pattern substantially as set forth in Panel K of FIG. 1 or FIG. 131K. In certain aspects, the present disclosure provides Form K of Formula (I) as characterized by an X-ray powder diffraction pattern comprising peaks at 5.3±0.3°2θ, 14.5±0.3°2θ, and 7.3±0.3°2θ. In some cases, the X-ray powder diffraction pattern further comprises at least one, at least two, at least three, at least four, or at least five peaks selected from 20.9±0.3°2θ, 3.4±0.3°2θ, 21.1±0.3°2θ, 7.4±0.3°2θ, and 14.8±0.3°2θ. In some cases, the X-ray powder diffraction pattern further comprises a peak at 17.4±0.3°2θ. In some cases, a relative intensity of the peak at 17.4±0.3°2θ is 30% to 80%, 35% to 75%, 40% to 70%, 45% to 65%, or 50% to 60% of the intensity of the peak at 5.3±0.3°2θ. In some cases, Formula (I) is at least 50%, at least 60%, at least 70%, at least 80%, at least 90%, at least 95%, at least 98%, at least 99%, at least 99.5%, or at least 99.9% Form K by weight.

Form K of Formula (I) can exhibit an X-ray powder diffraction pattern shown in FIG. 131K with peaks 1-36 corresponding to the peak numbers in TABLE 37. Peaks in FIG. 131K (numbered from 1 to 36 in order from left to right) and TABLE 37 are provided as relative intensities (Rel. Int. %) standardized against peak 2 (the peak with the highest intensity).

TABLE 37

| | Pos. | Height | FWHM | d-spacing | Rel. Int. |
|---|---|---|---|---|---|
| Peak | [°2θ] | [cts] | [°2θ] | [Å] | [%] |
| 1 | 3.3769 | 918.99 | 0.2362 | 26.16462 | 95.67 |
| 2 | 5.2629 | 960.61 | 0.1771 | 16.79195 | 100.00 |
| 3 | 7.2787 | 928.98 | 0.1181 | 12.14534 | 96.71 |
| 4 | 7.4150 | 888.24 | 0.0787 | 11.92233 | 92.47 |
| 5 | 9.5056 | 168.85 | 0.1574 | 9.30444 | 17.58 |
| 6 | 10.4555 | 275.13 | 0.0787 | 8.46112 | 28.64 |
| 7 | 12.2279 | 176.44 | 0.1968 | 7.23845 | 18.37 |
| 8 | 13.4369 | 160.94 | 0.1181 | 6.58973 | 16.75 |
| 9 | 13.7659 | 190.62 | 0.1181 | 6.43299 | 19.84 |
| 10 | 14.4554 | 941.33 | 0.0787 | 6.12763 | 97.99 |
| 11 | 14.8419 | 886.68 | 0.0787 | 5.96892 | 92.30 |
| 12 | 15.6218 | 293.09 | 0.0787 | 5.67266 | 30.51 |
| 13 | 16.4789 | 131.39 | 0.1574 | 5.37949 | 13.68 |
| 14 | 17.4049 | 531.15 | 0.1181 | 5.09532 | 55.29 |
| 15 | 18.0657 | 477.34 | 0.1378 | 4.91041 | 49.69 |
| 16 | 19.0583 | 360.24 | 0.1378 | 4.65684 | 37.50 |
| 17 | 19.9302 | 454.51 | 0.1181 | 4.45504 | 47.31 |
| 18 | 20.3915 | 214.70 | 0.1574 | 4.35529 | 22.35 |
| 19 | 20.9237 | 925.28 | 0.0984 | 4.24569 | 96.32 |
| 20 | 21.0590 | 910.40 | 0.0787 | 4.21872 | 94.77 |
| 21 | 22.2405 | 294.95 | 0.1574 | 3.99722 | 30.70 |
| 22 | 22.5573 | 229.33 | 0.1181 | 3.94178 | 23.87 |
| 23 | 23.8378 | 118.54 | 0.2362 | 3.73286 | 12.34 |
| 24 | 24.3835 | 387.82 | 0.0984 | 3.65055 | 40.37 |
| 25 | 24.8436 | 220.21 | 0.1574 | 3.58397 | 22.92 |
| 26 | 25.2484 | 180.33 | 0.1968 | 3.52741 | 18.77 |
| 27 | 27.2364 | 96.55 | 0.1574 | 3.27430 | 10.05 |
| 28 | 27.7899 | 95.20 | 0.3936 | 3.21033 | 9.91 |
| 29 | 29.3746 | 79.97 | 0.2362 | 3.04065 | 8.33 |
| 30 | 29.8805 | 99.99 | 0.2362 | 2.99032 | 10.41 |
| 31 | 31.6238 | 39.51 | 0.2362 | 2.82934 | 4.11 |
| 32 | 33.3928 | 48.52 | 0.1968 | 2.68338 | 5.05 |
| 33 | 35.5880 | 69.96 | 0.1574 | 2.52273 | 7.28 |
| 34 | 38.0252 | 18.17 | 0.4723 | 2.36646 | 1.89 |
| 35 | 39.5140 | 13.21 | 0.6298 | 2.28066 | 1.37 |
| 36 | 40.6789 | 24.98 | 0.4800 | 2.21617 | 2.60 |

In certain aspects, the present disclosure provides Form L of Formula (I) (hereinafter “Form L”) as characterized by an X-ray powder diffraction pattern substantially as set forth in Panel L of FIG. 1 or FIG. 131L. In certain aspects, the present disclosure provides Form L of Formula (I) as characterized by an X-ray powder diffraction pattern comprising peaks at 8.9±0.3°2θ, 3.4±0.3°2θ, and 18.3±0.3°2θ. In some cases, the peak at 8.9±0.3°2θ is about 10% to 45%, about 15% to 40%, about 20% to 35%, or about 23% to 31% more intense than the peak at 18.3±0.3°2θ. In some cases, the X-ray powder diffraction pattern further comprises at least one, at least two, or at least three peaks selected from 14.4±0.3°2θ, 21.9±0.3°2θ, and 18.0±0.3°2θ. In some cases, the X-ray powder diffraction pattern further comprises at least one, at least two, at least three, at least four, or at least five peaks selected from 14.3±0.3°2θ, 13.2±0.3°2θ, 20.0±0.3°2θ, 19.3±0.3°2θ, and 14.9±0.3°2θ. In some cases, Formula (I) is at least 50%, at least 60%, at least 70%, at least 80%, at least 90%, at least 95%, at least 98%, at least 99%, at least 99.5%, or at least 99.9% Form L by weight.

Form L of Formula (I) can exhibit an X-ray powder diffraction pattern shown in FIG. 131L with peaks 1-36 corresponding to the peak numbers in TABLE 38. Peaks in FIG. 131L (numbered from 1 to 36 in order from left to right) and TABLE 38 are provided as relative intensities (Rel. Int. %) standardized against peak 4 (the peak with the highest intensity).

TABLE 38

| Peak | Pos. [°2θ] | Height [cts] | FWHM [°2θ] | d-spacing [Å] | Rel. Int. [%] |
|---|---|---|---|---|---|
| 1 | 3.3973 | 919.48 | 0.1968 | 26.00787 | 98.17 |
| 2 | 5.4701 | 141.46 | 0.2362 | 16.15636 | 15.10 |
| 3 | 6.7617 | 137.89 | 0.1181 | 13.07274 | 14.72 |
| 4 | 8.9250 | 936.64 | 0.1574 | 9.90839 | 100.00 |
| 5 | 9.5163 | 183.05 | 0.2362 | 9.29400 | 19.54 |
| 6 | 9.9498 | 128.30 | 0.1574 | 8.89003 | 13.70 |
| 7 | 10.8198 | 289.71 | 0.0590 | 8.17708 | 30.93 |
| 8 | 12.0215 | 225.41 | 0.1181 | 7.36223 | 24.07 |
| 9 | 12.1866 | 255.53 | 0.0590 | 7.26285 | 27.28 |
| 10 | 12.5747 | 147.56 | 0.1574 | 7.03958 | 15.75 |
| 11 | 13.2489 | 494.74 | 0.2165 | 6.68281 | 52.82 |
| 12 | 13.4695 | 282.97 | 0.1181 | 6.57385 | 30.21 |
| 13 | 14.3045 | 513.68 | 0.0787 | 6.19197 | 54.84 |
| 14 | 14.4086 | 687.07 | 0.0984 | 6.14744 | 73.36 |
| 15 | 14.8811 | 400.16 | 0.1771 | 5.95328 | 42.72 |
| 16 | 15.2226 | 253.96 | 0.1574 | 5.82052 | 27.11 |
| 17 | 15.4349 | 191.73 | 0.1181 | 5.74091 | 20.47 |
| 18 | 16.1169 | 167.08 | 0.1574 | 5.49948 | 17.84 |
| 19 | 16.4526 | 257.19 | 0.0984 | 5.38803 | 27.46 |
| 20 | 16.8921 | 305.63 | 0.0984 | 5.24881 | 32.63 |
| 21 | 17.9743 | 531.26 | 0.0787 | 4.93517 | 56.72 |
| 22 | 18.3003 | 696.25 | 0.1181 | 4.84798 | 74.33 |
| 23 | 19.2521 | 414.41 | 0.0984 | 4.61040 | 44.24 |
| 24 | 20.0059 | 456.41 | 0.1771 | 4.43836 | 48.73 |
| 25 | 20.6642 | 192.05 | 0.3149 | 4.29842 | 20.50 |
| 26 | 21.2988 | 175.69 | 0.1574 | 4.17177 | 18.76 |
| 27 | 21.8881 | 543.65 | 0.1574 | 4.06076 | 58.04 |
| 28 | 22.3376 | 252.57 | 0.1574 | 3.98005 | 26.97 |
| 29 | 22.6117 | 240.29 | 0.1181 | 3.93243 | 25.65 |
| 30 | 23.6556 | 202.39 | 0.0984 | 3.76120 | 21.61 |
| 31 | 25.2162 | 227.40 | 0.1181 | 3.53185 | 24.28 |
| 32 | 26.5707 | 80.34 | 0.4723 | 3.35480 | 8.58 |
| 33 | 27.9435 | 58.75 | 0.4723 | 3.19303 | 6.27 |
| 34 | 30.6656 | 24.33 | 0.9446 | 2.91552 | 2.60 |
| 35 | 33.4929 | 12.73 | 0.4723 | 2.67559 | 1.36 |
| 36 | 34.7313 | 18.08 | 0.4723 | 2.58298 | 1.93 |

### Synthesis Methods

Aspects of the present disclosure provide methods for the synthetic preparation of (S)-1-(3-fluoro-4-(trifluoromethoxy)phenyl)-3-(1-(2-methylbutanoyl)piperidin-4-yl)urea (Formula (I)). Stable Formula (I) can be prepared in accordance with SCHEMES 1-2 as described below and in EXAMPLES 1-3.

### Synthetic Route 1

As disclosed herein, Formula (I) may be generated utilizing one, two, three, or all four of STEPS 1-4 of SCHEME 1.

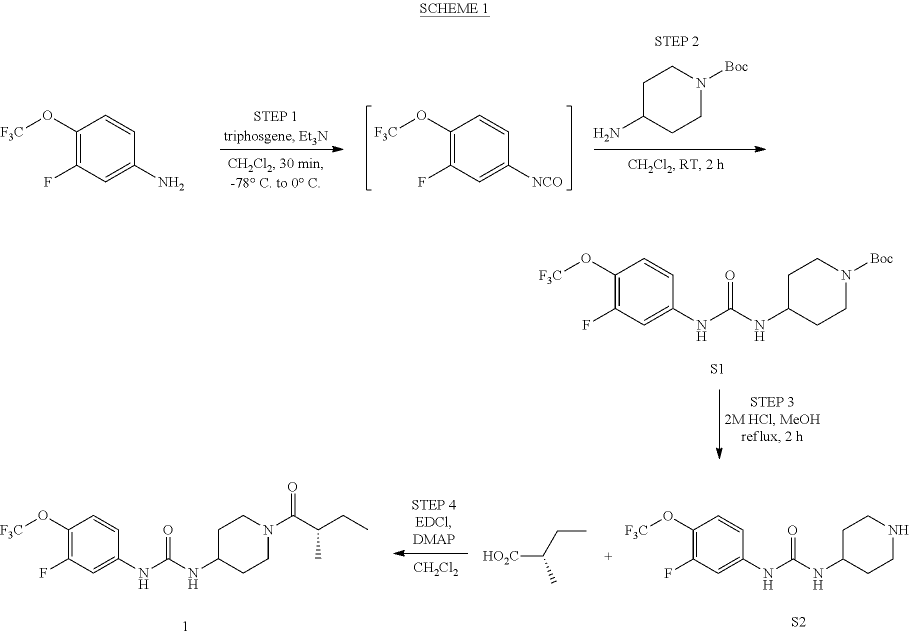

Step 1—Isocyanato Activation

Formula (I) synthesis can include 3-fluoro-4-(trifluoromethoxy)aniline activation to form the reactive isocyanide 2-fluoro-4-isocyanato-1-(trifluoromethoxy)benzene. In this step, 3-fluoro-4-(trifluoromethoxy)aniline and 1 triethylamine can be dissolved in CH₂Cl₂ and stirred at −78° C. Triphosgene can then be dissolved in CH₂Cl₂ and added dropwise. The reaction can be warmed to room temperature and stirred for 30 minutes before cooling to 0° C. The resultant isocyanato product can be used directly in subsequent synthesis steps.

### Step 2-Urea Formation

Formula (I) synthesis can include urea formation between 2-fluoro-4-isocyanato-1-(trifluoromethoxy)benzene and tert-butyl-4-aminopiperidine-1-carboxylate (a Boc-protected diamine) by combining these species with triethylamine (e.g., in approximately 2:3:3 ratio) in CH₂Cl₂ and stirring for 12 hours at room temperature. The reaction can be quenched with the addition of acid (e.g., 2 M HCl). Intermediate compound tert-butyl 4-(3-(3-fluoro-4-(trifluoromethoxy)phenyl)ureido)piperidine-1-carboxylate (Compound S1) can be collected from the organic layer and further extracted from the aqueous layer, and can optionally be dried and concentrated (e.g., in vacuo) for further use.

### Step 3—Deprotection

Formula (I) synthesis can include removal of a Boc protecting group from Compound S1 by refluxing in acid. In this step, Compound S1 can be dissolved (e.g., to a concentration of about 186 mM) in 2 M HCl in MeOH, and refluxed for 2 hours, forming 1-(3-fluoro-4-(trifluoromethoxy)phenyl)-3-(piperidin-4-yl)urea (Compound S2). Solvent can optionally be removed (e.g., in vacuo) and pH of the crude reaction product can be increased (e.g., to pH 12 with NaOH). The precipitate can be filtered, dried, and collected.

### Step 4—Piperidine Functionalization

Formula (I) synthesis can include functionalization of the piperidinyl amine in Compound S2 or an analogue thereof. This step can include activation of (S)-2-methylbutanoic acid with (S)-2-methylbutanoic acid (EDCl) and 4-Dimethylaminopyridine (DMAP) in the presence of Compound S2 in C H₂Cl₂. The reaction mixture can be stirred overnight at room temperature, and then quenched by addition of acid (e.g., 1M HCl). Resultant Formula (I) can be collected from the organic layer and extracted from the aqueous layer, and optionally can be subjected to further purification.

### Synthetic Route 2

As disclosed herein, Formula (I) may be generated utilizing one or both STEPS 1-2 of SCHEME 2.

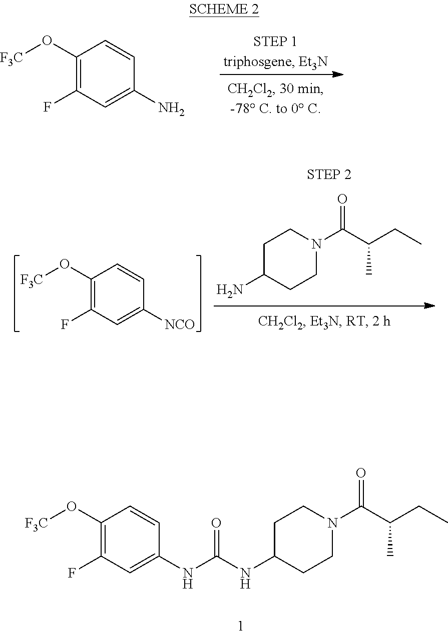

### Step 1—Isocyanato Activation

Formula (I) synthesis can include 3-fluoro-4-(trifluoromethoxy)aniline activation to form a reactive isocyanato as outlined for SCHEME 1 STEP 1 above. In this step, 3-fluoro-4-(trifluoromethoxy)aniline and 1 triethylamine can be dissolved in CH₂Cl₂, and combined with triphosgene to form 2-fluoro-4-isocyanato-1-(trifluoromethoxy)benzene.

### Step 2—Urea Formation

Formula (I) synthesis can include urea formation between 2-fluoro-4-isocyanato-1-(trifluoromethoxy)benzene and (S)-1-(4-aminopiperidin-1-yl)-2-methylbutan-1-one. This step can include combining these species with triethylamine (in CH₂Cl₂ and stirring at room temperature. The reaction can be quenched with the addition of acid (e.g., 2 M HCl). Product species Formula (I) can be collected from the organic layer and further extracted from the aqueous layer, and can optionally be dried, concentrated, and subjected to further purification.

### Methods for Making Polymorphs

A surprising discovery disclosed herein is that Formula (I) can be prepared in a variety of polymorphic forms, each with unique solubilities and physical properties. Among the determinants for Formula (I) forms, solvent system, temperature, cooling rate, and evaporation rate can affect the form of Formula (I) produced from a crystallization procedure. Often, a crystallization disclosed herein produces a single form of Formula (I), for example at least 90%, at least 95%, at least 98%, at least 99%, or at least 99.5% of the single form by weight. In some cases, a crystallization generates a negligible amount of amorphous Formula (I), for example less than 5%, less than 2%, less than 1%, or less than 0.5% by weight. In some cases, non-targeted forms of Formula (I) are selectively removed following crystallization, for example through triturating soluble impurities.

While Formula (I) is soluble in a range of solvents, solvent-type can strongly influence the form and purity of Formula (I) obtained from a crystallization. In some cases, the primary solvent for crystallization is an organic solvent. In some cases, the organic solvent is a protic organic solvent. In other cases, the organic solvent is aprotic. In some cases, the organic solvent is acetone, acetonitrile, dichloromethane, dioxane, isopropyl alcohol, isopropyl acetate, methanol, methylethylketone, methyl isobutyl ketone, methyl tert-butyl ether, n-butyl alcohol, tetrahydrofuran, 2-methyl tetrahydrofuran, toluene, or a combination thereof. In some cases, Formula (I) comprises a solubility of at least about 5 mg/mL, at least about 10 mg/mL, at least about 20 mg/mL, at least about 30 mg/mL, at least about 40 mg/mL, or at least about 50 mg/mL in the primary solvent (e.g., as outlined in TABLE 4 for Form A).

A Formula (I) crystallization can utilize a single solvent or a solvent mixture. In many cases, Formula (I) crystallization utilizes a solvent system with at least two solvents. In some cases, two solvents used for crystallization are miscible, for example methanol and water. In some cases, Formula (I) has a solubility of at least 5 mg/mL in a secondary solvent. However, in many cases, Formula (I) crystallizations utilize a primary solvent with a high Formula (I) solubility and an antisolvent with a low Formula (I) solubility. As used herein, the term “antisolvent” can denote a solvent in which an analyte (e.g., Formula (I)) has lower solubility. In some cases, Formula (I) has a solubility of at most 5 mg/mL, at most 3 mg/mL, at most 2 mg/mL, or at most 1 mg/mL in a secondary solvent. In some cases, the secondary solvent is water or a hexane. In some cases, a solvent system used for Formula (I) crystallization only contains solvents in which Formula (I) has at least 5 mg/ml, 10 mg/ml, or 20 mg/ml solubility. In some cases, a solvent system used for Formula (I) crystallization contains a first solvent in which Formula (I) has at least 10 mg/ml solubility and a second solvent in which Formula (I) has at most 5 mg/ml solubility at room temperature. In some cases, a solvent system used for Formula (I) crystallization contains a first solvent in which Formula (I) has at least 20 mg/ml solubility and a second solvent in which Formula (I) has at most 2 mg/ml solubility at room temperature.

In some cases, a Formula (I) crystallization utilizes a multi-solvent system comprising water and an organic solvent. In some cases, the organic solvent is selected from the group consisting of methanol, ethanol, acetonitrile, isopropyl alcohol, tetrahydrofuran, acetone, dimethyl sulfoxide, dimethyl formamide, NMP, n-propanol, and dioxane. In some cases, the organic solvent is selected from the group consisting of ethanol, acetonitrile, isopropyl alcohol, tetrahydrofuran, acetone, dimethyl sulfoxide, dimethyl formamide, NMP, n-propanol, and dioxane. In some cases, the water and organic solvent are in a ratio of between 10:1 and 1:10, between 10:1 and 1:1, between 5:1 and 1:5, between 5:2 and 2:5, between 3:2 and 2:3, or between 1:1 and 1:10.

In some cases, a Formula (I) crystallization comprises a heptane and an additional organic solvent. In some cases, the heptane is n-heptane. In some cases, the organic solvent is selected from the group consisting of methanol, ethanol, acetonitrile, isopropyl alcohol, tetrahydrofuran, acetone, dimethyl sulfoxide, dimethyl formamide, NMP, n-propanol, and dioxane. In some cases, a ratio of heptane to methanol, ethanol, acetonitrile, isopropyl alcohol, dimethyl sulfoxide, NMP, n-propanol, or dioxane is between 5:1 and 200:1, between 10:1 and 200:1, between 5:1 and 100:1, or between 10:1 and 300:1. In some cases, a ratio of heptane to tetrahydrofuran or acetone is between 20:1 and 1:1, between 10:1 and 1:1, between 5:1 and 1:5, or between 5:1 and 1:1.

In some cases, a Formula (I) crystallization comprises a hexane and an additional organic solvent. In some cases, the hexane is c-hexane. In some cases, the organic solvent is selected from the group consisting of methanol, ethanol, acetonitrile, isopropyl alcohol, tetrahydrofuran, acetone, dimethyl sulfoxide, dimethyl formamide, NMP, n-propanol, and dioxane. In some cases, a ratio of hexane to methanol, ethanol, acetonitrile, isopropyl alcohol, dimethyl sulfoxide, NMP, or n-propanol is between 5:1 and 200:1, between 10:1 and 200:1, between 5:1 and 100:1, or between 10:1 and 300:1. In some cases, a ratio of hexane to tetrahydrofuran, acetone, or dioxane is between 20:1 and 1:1, between 10:1 and 1:1, between 5:1 and 1:5, or between 5:1 and 1:1.

In some cases, Formula (I) is dissolved at a temperature of at least 30° C. and then cooled for crystallization. In some cases, Formula (I) is dissolved at a temperature of at least 40° C., at least 50° C., at least 60° C., or at least 70° C. and then cooled for crystallization. In some cases, Formula (I) is dissolved at a temperature of at most 70° C., at most 60° C., at most 50° C., at most 40° C., or at most 30° C. and then cooled for crystallization. In some cases, Formula (I) is dissolved at a temperature of between about 30° C. and 90° C., between about 40° C. and 80° C., or about 50° C. and 75° C. and then cooled for crystallization.

The rate at which a Formula (I) solution is cooled following its dissolution in a solvent system can affect which polymorph(s) are generated during crystallization. The cooling can be slow, for example at most about 0.1° C./hour, at most about 1° C./hour, at most about 2° C./hour, at most about 4° C./hour, at most about 8° C./hour, at most about 12° C./hour, at most about 15° C./hour, at most about 20° C./hour, at most about 25° C./hour, at most about 30° C./hour, or at most about 40° C./hour. The cooling rate can be between about 0.1° C./hour and 40° C./hour, between about 1° C./hour and about 40° C./hour, between about 4° C./hour and 20° C./hour, between about 4° C./hour and 30° C./hour, between about 8° C./hour and 25° C./hour, or between about 15° C./hour and 40° C./hour per hour. The cooling rate can also be fast, for example between 60° C./hour and 600° C./hour, greater than about 60° C./hour, greater than about 100° C./hour, or greater than about 200° C./hour. In some cases, the cooling lowers the temperature of the solvent system below 30° C. In some cases, the cooling lowers the temperature of the solvent system below 27° C. In some cases, the cooling lowers the temperature of the solvent system below 20° C. In some cases, the cooling lowers the temperature of the solvent system below 10° C. In some cases, the cooling lowers the temperature of the solvent system below 5° C.

In some cases, Formula (I) is added to a solvent system at a first temperature, and then cooled to a second temperature at which Formula (I) has a lower solubility in the solvent system. In some cases, the solvent system is saturated with Formula (I) at the first temperature. In some cases, Formula (I) is added to about 60-90% saturation to the solvent system at the first temperature. In some cases, Formula (I) is added to about 40-80% saturation to the solvent system at the first temperature. In some cases, Formula (I) is added to about 30-60% saturation to the solvent system at the first temperature. In some cases, Formula (I) is added to about 75% to greater than 100% saturation at the first temperature.

Following dissolution, a solvent system containing Formula (I) can be seeded with solid Formula (I). In some cases, the Formula (I) is of a single polymorphic form. In some cases, the Formula (I) is of Form A, B, C, D, E, F, G, H, I, J, K, or L. In some cases, the Formula (I) is added at a milligram scale, for example 1-5 mg.

Crystallization can also include addition of a low Formula (I)-solubility solvent (e.g., an antisolvent). The added solvent can have a lower Formula (I) solubility than the primary solvent used for crystallization, such that its addition lowers the solubility of Formula (I) within the solvent system. In many cases, a crystallization method can include the gradual addition of antisolvent (e.g., water) into a solvent system with a primary solvent in which Formula (I) comprises a high solubility. For example, certain crystallization methods disclosed herein include the addition of water, n-heptane, c-heptane, or another solvent in which Formula (I) has a solubility of less than about 5 mg/ml in conditions used for a crystallization.

In some cases, Formula (I) is added to a solvent system prior to addition of an antisolvent. In some cases, the solvent system is saturated with Formula (I) prior to the addition of the antisolvent. In some cases, Formula (I) is added to about 60-90% saturation to the solvent system prior to the addition of the antisolvent. In some cases, Formula (I) is added to about 40-80% saturation to the solvent system prior to the addition of the antisolvent. In some cases, Formula (I) is added to about 30-60% saturation to the solvent system prior to the addition of the antisolvent. In some cases, Formula (I) is added to about 75% to greater than 100% saturation prior to the addition of the antisolvent.

### (i) Methods for Making Form A

In some cases, Form A is formed during fast cooling in a single-solvent or substantially single-solvent system. As used herein, a single-solvent or substantially single-solvent system can include at least 90%, at least 95%, or at least 99% of a primary solvent. In some cases, the solvent is methanol or toluene.

In some cases, Form A is formed during slow cooling in a multi-solvent system (e.g., a solvent system comprising at least 90%, at least 95%, or at least 99% of the specified solvents by volume). In some cases, the solvent system includes water and a solvent selected from the group consisting of acetonitrile acetone.

In some cases, Form A is formed during fast cooling in a multi-solvent system. In some cases, the solvent system includes water, acetonitrile, and at least 0.25 mg/ml Formula (I) in the multi-solvent system.

In some cases, Form A is formed by heating Form C. As shown in TABLE 15, Form C completes a 100% conversion to Form A in less than 1 day when incubated at 60° C. A method for making Form A can include incubating Form C at a temperature of at least 40° C., at least 50° C., at least 60° C., or between 40° C. and 90° C. for at least 1 hour, at least 6 hours, at least 12 hours, or at least 1 day. The incubation may be performed in low humidity or strictly anhydrous conditions.

### (ii) Methods for Making Form B

In some cases, Form B is formed during slow cooling in a single-solvent or substantially single-solvent. In some cases, the single solvent is methanol, ethanol, isopropyl alcohol, or n-butanol.

In some cases, Form B is formed during fast cooling in a single-solvent or substantially single-solvent system. In some cases, the solvent is ethanol, isopropyl alcohol, or n-butanol.

In some cases, Form B is formed during fast cooling in a multi-solvent system. In some cases, the solvent system includes water and n-propanol.

In some cases, Form B is formed during slow cooling in a multi-solvent system. In some cases, the multi-solvent system includes an organic solvent in which Formula (I) has less than 5 mg/ml solubility at room temperature. In some cases, the multi-solvent system includes an alkane in which Formula (I) has less than 5 mg/ml solubility at room temperature. In some cases, the multi-solvent system includes c-hexane and dioxane. In some cases, the multi-solvent system includes c-hexane and acetonitrile, and the crystallization utilizes at most about 0.1 mg/ml Formula (I).

(iii) Methods for Making Form C

In some cases, Form C is formed during fast cooling in a multi-solvent system. In some cases, the solvent system includes water and methanol.

In some cases, Form C is formed by incubating a Formula (I) polymorph in water. In some cases, the Formula (I) polymorph is one or more of Forms A-L. In some cases, the incubation is performed at or near room temperature (e.g., between 15° C. and 35° C.). In some cases, the incubation is performed for at least 1 hour, at least 6 hours, at least 12 hours, at least 1 day, at least 2 days, or at least 5 days. In some cases, the Formula (I) polymorph is an anhydrous polymorph.

In some cases, Form C is formed by incubating a Formula (I) polymorph in a polyethylene glycol (PEG):water mixture containing at least 50% water by volume. In some cases, the PEG has a molecular weight of at least about 50, at least about 100, at least about 200, at least about 300, at least about 500, or at least about 1000. In some cases, the incubation is performed for at least 1 hour, at least 6 hours, at least 12 hours, at least 1 day, at least 2 days, or at least 5 days. In some cases, the Formula (I) polymorph is an anhydrous polymorph.

In some cases, Form C is formed by incubating another Form of Formula (I) in a micellular system. In some cases, the micellular system includes a nonionic surfactant. In some cases, the non-ionic surfactant is polysorbate 80 (“Tween 80”) or sodium lauryl sulfate. In some cases, the critical micelle concentration of the micellular system is between about 0.1 and 150 or between about 1 and 100.

### (iv) Methods for Making Form D

In some cases, Form D is formed during fast cooling in a multi-solvent system. In some cases, the solvent system includes an organic solvent in which Formula (I) has less than 5 mg/ml solubility at room temperature and a solvent selected from the group consisting of dimethyl sulfoxide and NMP. In some cases, the organic solvent in which Formula (I) has less than 5 mg/ml solubility at room temperature is n-heptane.

In some cases, Form D is formed during slow cooling in a multi-solvent system. In some cases, the solvent system includes water and dimethyl formamide, and the crystallization utilizes at most about 0.25 mg/ml Formula (I). In some cases, the solvent system includes an organic solvent in which Formula (I) has less than 5 mg/ml solubility at room temperature. In some cases, the solvent system includes n-heptane and a solvent selected from the group consisting of dimethyl formamide and NM P. In some cases, the solvent system includes n-heptane, isopropyl alcohol, and at least about 0.4 mg/ml Formula (I) are utilized for crystallization. In some cases, the solvent system includes n-heptane and a solvent selected from the group consisting of ethanol, isopropyl alcohol, and ethyl acetate, and the crystallization includes seeding with Form D crystals. In some cases, the solvent system includes c-hexane and dimethyl sulfoxide.

In some cases, Form D is formed by incubation of a Formula (I) polymorph in n-heptane. In some cases, the Formula (I) polymorph is one or more of Forms A-L. In some cases, the incubation is performed at or near room temperature (e.g., between 15° C. and 35° C.). In some cases, the incubation is performed for more than one day.

In some cases, Form D is formed by heating Form B. For example, as outlined in TABLE 15, Form B rapidly converts to Form D at 60° C., with 100% conversion occurring following one day of incubation. Following from this observation, a method for making Form D can include incubating Form B at a temperature of at least 40° C., at least 50° C., or at least 60° C. for at least 1 hour, at least 6 hours, at least 12 hours, or at least 1 day.

In some cases, Form D is formed by incubating Form C in a PEG:water mixture containing greater than 50% PEG by volume. In some cases, the mixture contains at least 60% PEG, at least 70% PEG, at least 75% PEG, at least 80% PEG, or at least 90% PEG by volume. In some cases, the PEG has a molecular weight of at least about 50, at least about 100, at least about 200, at least about 300, at least about 500, or at least about 1000.

### (v) Methods for Making Form E

In some cases, Form E is formed during fast cooling in a multi-solvent system. In some cases, the solvent system includes an organic solvent in which Formula (I) has less than 5 mg/ml solubility at room temperature and a solvent selected from the group consisting of methanol, ethanol, acetonitrile, isopropyl alcohol, acetone, and n-propanol. In some cases, the organic solvent in which Formula (I) has less than 5 mg/ml solubility at room temperature is n-heptane.

In some cases, Form E is formed during slow cooling in a multi-solvent system. In some cases, the solvent system includes an organic solvent in which Formula (I) has less than 5 mg/ml solubility at room temperature. In some cases, the solvent system includes n-heptane and a solvent selected from the group consisting of methanol, ethanol, acetonitrile, and n-propanol. In some cases, the solvent system includes n-heptane and methyl ethyl ketone, and the crystallization includes seeding with Form E crystals. In some cases, the solvent system includes c-hexane and methanol. In some cases, the solvent system includes n-heptane and isopropyl alcohol, and the crystallization utilizes at most about 0.25 mg/ml Formula (I).

For many slurry-based conversions disclosed herein, non-Form D first converts to Form E and then subsequently converts to Form D during incubation in a heptane for less than one day. In some cases, the heptane is n-heptane. In some cases, the Formula (I) polymorph is one or more of Forms A-C or E-L. In some cases, the incubation is performed at or near room temperature (e.g., between 15° C. and 35° C.). In some cases, the incubation is performed for at most one day.

### (vi) Methods for Making Form F

In some cases, Form F is formed during fast cooling in a multi-solvent system. In some cases, the solvent system includes water and a solvent selected from the group consisting of methanol, ethanol, isopropyl alcohol, tetrahydrofuran, acetone, dimethyl sulfoxide, dimethyl formamide, N-Methyl and -2-pyrrolidone (NMP). In some cases, the solvent system includes water and acetonitrile, and the crystallization utilizes at most about 0.25 mg/ml Formula (I). In some cases, the solvent system includes an organic solvent in which Formula (I) has less than 5 mg/ml solubility at room temperature. In some cases, the solvent system includes n-heptane and dimethylformamide. In some cases, the solvent system includes c-hexane and a solvent selected from the group consisting of ethanol, NMP, n-propanol, and dioxane.

In some cases, Form F is formed during slow cooling in a multi-solvent system. In some cases, the solvent system includes water and a solvent selected from the group consisting of ethanol, tetrahydrofuran, dimethylformamide, and n-propanol. In some cases, the solvent system includes an organic solvent in which Formula (I) has less than 5 mg/ml solubility at room temperature. In some cases, solvent system includes n-heptane and dimethyl sulfoxide. In some cases, the solvent system includes c-hexane and a solvent selected from the group consisting of methanol, isopropyl alcohol, dimethyl formamide, and n-propanol.

(vii) Methods for Making Form G

In some cases, Form G is formed during slow cooling in a single-solvent or substantially single-solvent system. In some cases, the single solvent is tetrahydrofuran (THF), methyl ethyl ketone (MEK), or dioxane.

In some cases, Form G is formed during fast cooling in a single-solvent or substantially single-solvent system. In some cases, the solvent is tetrahydrofuran or dioxane.

In some cases, Form G is formed during fast cooling in a multi-solvent system. In some cases, the solvent system includes water and dioxane. In some cases, the solvent system includes an organic solvent in which Formula (I) has less than 5 mg/ml solubility at room temperature. In some cases, the solvent system includes n-heptane and a solvent selected from the group consisting of tetrahydrofuran and dioxane. In some cases, the solvent system includes c-hexane and acetone, and the crystallization utilizes at most about 0.25 mg/ml Formula (I).

In some cases, Form G is formed during slow cooling in a multi-solvent system. In some cases, the solvent system includes water and dioxane. In some cases, the solvent system includes an organic solvent in which Formula (I) has less than 5 mg/ml solubility at room temperature. In some cases, the solvent system includes n-heptane and dioxane. In some cases, the solvent system includes c-hexane and tetrahydrofuran. In some cases, the solvent system includes c-hexane, dioxane, and at least about 0.4 mg/ml Formula (I) in the solvent system.

(viii) Methods for Making Form H

In some cases, Form H is formed during slow cooling in a single-solvent or substantially single-solvent system. In some cases, the single solvent is 2-methyl tetrahydrofuran (2-MeTHF), isopropyl acetate (IPAc), or methyl isobutyl ketone (MIBK).

In some cases, Form H is formed during fast cooling in a single-solvent or substantially single-solvent system. In some cases, the solvent is isopropyl acetate or 2-methyl tetrahydrofuran.

In some cases, Form H is formed during fast cooling in a multi-solvent system. In some cases, the multi-solvent system includes an organic solvent in which Formula (I) has less than 5 mg/ml solubility at room temperature. In some cases, the multi-solvent system includes c-hexane and a solvent selected from the group consisting of acetonitrile and tetrahydrofuran.

In some cases, the solvent system includes c-hexane and acetone, and at least about 0.4 mg/ml Formula (I) in the solvent system.

In some cases, Form H is formed during slow cooling in a multi-solvent system. In some cases, the multi-solvent system includes an organic solvent in which Formula (I) has less than 5 mg/ml solubility at room temperature. In some cases, the multi-solvent system includes c-hexane and acetone. In some cases, the multi-solvent system includes c-hexane and acetonitrile, and at least about 0.4 mg/ml of Formula (I) are in the solvent system.

### (ix) Methods for Making Form I

In some cases, Form I is formed during slow cooling in a single-solvent or substantially single-solvent system. In some cases, the single solvent is dichloromethane.

In some cases, Form I is formed during fast cooling in a single-solvent or substantially single-solvent system. In some cases, the solvent is dichloromethane. In some cases, the solvent is acetonitrile, and the crystallization utilizes at most about 0.25 mg/ml Formula (I).

### (x) Methods for Making Form J

In some cases, Form J is formed during slow cooling in a single-solvent or substantially single-solvent system. In some cases, the single solvent is toluene.

### (xi) Methods for Making Form K

In some cases, Form K is formed during slow cooling in a single-solvent or substantially single-solvent system. In some cases, the single solvent is methyl tert-butyl ether (MTBE).

In some cases, Form K is formed during fast cooling in a single-solvent or substantially single-solvent system. In some cases, the single solvent is methyl tert-butyl ether (MTBE).

In some cases, Form K is formed by incubation of a Formula (I) polymorph in methyl tert-butyl ether. In some cases, the Formula (I) polymorph is one or more of Forms A-L. In some cases, the incubation is performed at or near room temperature (e.g., between 15° C. and 35° C.). In some cases, the incubation is performed for at least one day.

(xii) Methods for Making Form L

In some cases, Form L is formed during slow cooling in a multi-solvent system. In some cases, the multi-solvent system includes water and isopropyl alcohol, and at least about 0.4 mg/mL of Formula (I) are utilized for crystallization. In some cases, the multi-solvent system includes c-hexane and acetonitrile, and at least about 0.2 mg/mL Formula (I) are utilized for crystallization.

In some cases, Form L is formed during fast cooling in a multi-solvent system. In some cases, the solvent system includes an organic solvent in which Formula (I) has less than 5 mg/ml solubility at room temperature. In some cases, the solvent system includes n-heptane and tetrahydrofuran.

### Formulations

Formula (I) can be formulated for a range of delivery routes, including oral, parenteral (including subcutaneous, intradermal, intramuscular, intravenous, intraarticular, and intramedullary), intraperitoneal, transmucosal, transdermal, rectal and topical (including dermal, buccal, sublingual and intraocular) administration. As disclosed herein, Formula (I) can be prepared in numerous polymorphic forms, each with unique solubilities, stabilities, and activities. Following from these discoveries, Formula (I) formulations can not only be tailored for a particular delivery route, but also for optimized suitability of the form of Formula (I) with a particular delivery system. For example, as outlined in EXAMPLE 17, solubility and stability in PEG-water mixtures can be enhanced through proper selection of Formula (I) polymorphic forms. Accordingly, optimal formulation not only includes delivery form (e.g., solid pill or micellular suspension), but also on the selection Formula (I) polymorph for the delivery form and desired activity level.

Formula (I) can be formulated raw or as a component of a pharmaceutical formulation. In many cases, a Formula (I) formulation includes one or more pharmaceutically acceptable carriers. As used herein, a pharmaceutically acceptable carrier can denote a non-therapeutically active ingredient which is compatible with the other ingredients of a pharmaceutical formulation. The pharmaceutical compositions disclosed herein may be manufactured in any manner known in the art, for example by means of conventional mixing, dissolving, granulating, dragee-making, levigating, emulsifying, encapsulating, entrapping or compression processes. A pharmaceutically acceptable carrier can be a solid, a liquid, an emulsifier, or a combination thereof. In some cases, a Formula (I) formulation includes polyethylene glycol, polypropylene glyocol, sulfobutylether, vitamin E, castor oil, hydrogenated castor oil, soybean oil, corn oil, canola oil, Miglyol 810, Miglyol 812, Capmul MCM, Kolliphor P188, Kolliphor EL, oleic acid, plurololeique, pecceol, labrasol, labrafil M 1944CS, Felucire 44/14, Captex 355, Plurol Oleique CC 497, triacetin, transcutol H P, glycerol, intralipid, or a combination thereof. In some cases, a Formula (I) formulation includes a surfactant. In some cases, the surfactant is an ionic surfactant. In some cases, the surfactant is a non-ionic surfactant.

Formula (I) size can be an important determinant of Formula (I) properties (e.g., dissolution rate in a particular solvent). Within a formulation, can Formula (I) can have size uniformity or be polydisperse. In some cases, Formula (I) particle size (e.g., crystal diameter) standard deviation is about 0.1-times the mean particle size, about 0.2-times the mean particle size, about 0.4-times the mean particle size, about 0.75-times the mean particle size, about equal to the mean particle size, or greater than the mean particle size. For some formulations disclosed herein, Formula (I) has a mean particle size (e.g., mean diameter of an individual crystal) of between about 0.1 and 500 microns. In some cases, Formula (I) has a mean particle size of between about 10 and 50 microns, between about 10 and 100 microns, between about 10 and 200 microns, between about 20 and 100 microns, between about 50 and 100 microns, between about 50 and 250 microns, or between about 100 and about 500 microns. In some cases, Formula (I) has a mean particle size of between about 0.1 and 25 microns, between about 0.1 and 0.5 microns, between about 0.1 and 1 microns, between about 0.1 and 2 microns, between about 0.25 and 1 microns, between about 0.25 and 2 microns, between about 0.25 and 4 microns, between about 0.5 and 2 microns, between about 0.5 and 5 microns, between about 1 and 5 microns, between about 2 and 6 microns, between between about 2 and 9 microns, between about 2 and 12 microns, between about 4 and 12 microns, between about 4 and 20 microns, between about 6 and 12 microns, between about 6 and 20 microns, between about 8 and 25 microns, or between about 10 and 30 microns.

The most suitable route may depend upon for example the condition and disorder of the recipient. The formulations may conveniently be presented in unit dosage form and may be prepared by any of the methods known in pharmaceutical formulation. Typically, these methods include the step of bringing into association a compound of the subject invention or a pharmaceutically acceptable salt, ester, amide, prodrug or solvate thereof (“active ingredient”) with a carrier which constitutes one or more accessory ingredients. In general, the formulations are prepared by uniformly and intimately bringing into association the active ingredient with liquid carriers or finely divided solid carriers or both and then, if necessary, shaping the product into the desired formulation.

Formulations of the compounds disclosed herein suitable for oral administration may be presented as discrete units such as capsules, cachets or tablets each containing a predetermined amount of the active ingredient; as a powder or granules; as a solution or a suspension in an aqueous liquid or a non-aqueous liquid; or as an oil-in-water liquid emulsion or a water-in-oil liquid emulsion. The active ingredient may also be presented as a bolus, electuary or paste.

Pharmaceutical preparations which can be used orally include tablets, push fit capsules made of gelatin, as well as soft, sealed capsules made of gelatin and a plasticizer, such as glycerol or sorbitol. Tablets may be made by compression or molding, optionally with one or more accessory ingredients. Compressed tablets may be prepared by compressing in a suitable machine the active ingredient in a free-flowing form such as a powder or granules, optionally mixed with binders, inert diluents, or lubricating, surface active or dispersing agents. Molded tablets may be made by molding in a suitable machine a mixture of the powdered compound moistened with an inert liquid diluent. The tablets may optionally be coated or scored and may be formulated so as to provide slow or controlled release of the active ingredient therein. All formulations for oral administration should be in dosages suitable for such administration. The push fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols. In addition, stabilizers may beaded. Dragee cores are provided with suitable coatings. For this purpose, concentrated sugar solutions may be used, which may optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments may be added to the tablets or dragee coatings for identification or to characterize different combinations of active compound doses.

The compounds may be formulated for parenteral administration by injection, e.g., by bolus injection or continuous infusion. Formulations for injection may be presented in unit dosage form, e.g., in ampoules or in multi dose containers, with an added preservative. The compositions may take such forms as suspensions, solutions or emulsions in oily or aqueous vehicles, and may contain formulatory agents such as suspending, stabilizing and/or dispersing agents. The formulations may be presented in unit-dose or multi-dose containers, for example sealed ampoules and vials, and may be stored in powder form or in a freeze-dried (lyophilized) condition requiring only the addition of the sterile liquid carrier, for example, saline or sterile pyrogen-free water, immediately prior to use. Extemporaneous injection solutions and suspensions may be prepared from sterile powders, granules and tablets of the kind previously described.

Formulations for parenteral administration include aqueous and non-aqueous (oily) sterile injection solutions of the active compounds which may contain antioxidants, buffers, bacteriostats and solutes which render the formulation isotonic with the blood of the intended recipient; and aqueous and non-aqueous sterile suspensions which may include suspending agents and thickening agents. Suitable lipophilic solvents or vehicles include fatty oils such as sesame oil, or synthetic fatty acid esters, such as ethyl oleate or triglycerides, or liposomes. Aqueous injection suspensions may contain substances which increase the viscosity of the suspension, such as sodium carboxymethyl cellulose, sorbitol, or dextran. Optionally, the suspension may also contain suitable stabilizers or agents which increase the solubility of the compounds to allow for the preparation of highly concentrated solutions.

In addition to the formulations described previously, the compounds may also be formulated as a depot preparation. Such long-acting formulations may be administered by implantation (for example subcutaneously or intramuscularly) or by intramuscular injection. Thus, for example, the compounds may be formulated with suitable polymeric or hydrophobic materials (for example as an emulsion in an acceptable oil) or ion exchange resins, or as sparingly soluble derivatives, for example, as a sparingly soluble salt.

For buccal or sublingual administration, the compositions may take the form of tablets, lozenges, pastilles, or gels formulated in conventional manner. Such compositions may comprise the active ingredient in a flavored basis such as sucrose and acacia or tragacanth.

Formula (I) may also be formulated in rectal compositions such as suppositories or retention enemas, e.g., containing conventional suppository bases such as cocoa butter, polyethylene glycol, or other glycerides.

Formula (I) can be formulated for topical administration, that is by non-systemic administration. This includes the application of a compound disclosed herein externally to the epidermis or the buccal cavity and the instillation of such a compound into the ear, eye and nose, such that the compound does not significantly enter the blood stream. In contrast, systemic administration refers to oral, intravenous, intraperitoneal and intramuscular administration. Formulations suitable for topical administration include liquid or semi-liquid preparations suitable for penetration through the skin to the site of inflammation such as gels, liniments, lotions, creams, ointments or pastes, and drops suitable for administration to the eye, ear or nose. The active ingredient for topical administration may comprise, for example, from 0.001% to 10% w/w (by weight) of the formulation. In certain embodiments, the active ingredient may comprise as much as 10% w/w. In other embodiments, it may comprise less than 5% w/w. In certain embodiments, the active ingredient may comprise from 2% w/w to 5% w/w. In other embodiments, it may comprise from 0.1% to 1% w/w of the formulation.

Topical ophthalmic, otic, and nasal formulations of the present invention may comprise excipients in addition to the active ingredient. Excipients commonly used in such formulations include, but are not limited to, tonicity agents, preservatives, chelating agents, buffering agents, and surfactants. Other excipients comprise solubilizing agents, stabilizing agents, comfort-enhancing agents, polymers, emollients, pH-adjusting agents and/or lubricants. Any of a variety of excipients may be used in formulations of the present invention including water, mixtures of water and water-miscible solvents, such as C1-C7-alkanols, vegetable oils or mineral oils comprising from 0.5 to 5% non-toxic water-soluble polymers, natural products, such as alginates, pectins, tragacanth, karaya gum, guar gum, xanthan gum, carrageenin, agar and acacia, starch derivatives, such as starch acetate and hydroxypropyl starch, and also other synthetic products such as polyvinyl alcohol, polyvinylpyrrolidone, polyvinyl methyl ether, polyethylene oxide, preferably cross-linked polyacrylic acid and mixtures of those products. The concentration of the excipient is, typically, from 1 to 100,000 times the concentration of the active ingredient. In preferred embodiments, the excipients to be included in the formulations are typically selected on the basis of their inertness towards the active ingredient component of the formulations.

Relative to ophthalmic, otic, and nasal formulations, suitable tonicity-adjusting agents include, but are not limited to, mannitol, sodium chloride, glycerin, sorbitol and the like. Suitable buffering agents include, but are not limited to, phosphates, borates, acetates and the like. Suitable surfactants include, but are not limited to, ionic and nonionic surfactants (though nonionic surfactants are preferred), RLM 100, POE 20 cetylstearyl ethers such as Procol® CS20 and poloxamers such as Pluronic® F68.

The formulations set forth herein may comprise one or more preservatives. Examples of such preservatives include p-hydroxybenzoic acid ester, sodium perborate, sodium chlorite, alcohols such as chlorobutanol, benzyl alcohol or phenyl ethanol, guanidine derivatives such as polyhexamethylene biguanide, sodium perborate, polyquaternium-1, amino alcohols such as AMP-95, or sorbic acid. In certain embodiments, the formulation may be self-preserved so that no preservation agent is required.

For ophthalmic, otic, or nasal administration, the formulation may be a solution, a suspension, or a gel. In preferred aspects, the formulations are for topical application to the eye, nose, or ear in aqueous solution in the form of drops. The term “aqueous” typically denotes an aqueous formulation wherein the formulation is >50%, more preferably >75% and in particular >90% by weight water. These drops may be delivered from a single dose ampoule which may preferably be sterile and thus render bacteriostatic components of the formulation unnecessary. Alternatively, the drops may be delivered from a multi-dose bottle which may preferably comprise a device which extracts any preservative from the formulation as it is delivered, such devices being known in the art. [0159] For ophthalmic disorders, components of the invention may be delivered to the eye as a concentrated gel or a similar vehicle, or as dissolvable inserts that are placed beneath the eyelids.

The formulations of the present invention that are adapted for topical administration to the eye are preferably isotonic, or slightly hypotonic in order to combat any hypertonicity of tears caused by evaporation and/or disease. This may require a tonicity agent to bring the osmolality of the formulation to a level at or near 210-320 milliosmoles per kilogram (mOsm/kg). The formulations of the present invention generally have an osmolality in the range of 220-320 mOsm/kg, and preferably have an osmolality in the range of 235-300 mOsm/kg. The ophthalmic formulations will generally be formulated as sterile aqueous solutions.

In certain ophthalmic embodiments, the compositions of the present invention are formulated with one or more tear substitutes. A variety of tear substitutes are known in the art and include, but are not limited to: monomeric polyols, such as, glycerol, propylene glycol, and ethylene glycol; polymeric polyols such as polyethylene glycol; cellulose esters such hydroxypropylmethyl cellulose, carboxy methylcellulose sodium and hydroxy propylcellulose; dextrans such as dextran 70; vinyl polymers, such as polyvinyl alcohol; and carbomers, such as carbomer 934P, carbomer 941, carbomer 940 and carbomer 974P. Certain formulations of the present invention may be used with contact lenses or other ophthalmic products. Preferred tear substituted formulations are prepared using a buffering system that maintains the formulation at a pH of about 4.5 to a pH of about 8. A most preferred formulation pH is from 6 to 8.

Gels for topical or transdermal administration may comprise, generally, a mixture of volatile solvents, nonvolatile solvents, and water. In certain embodiments, the volatile solvent component of the buffered solvent system may include lower (Ci-C6) alkyl alcohols, lower alkyl glycols and lower glycol polymers. In further embodiments, the volatile solvent is ethanol. The volatile solvent component is thought to act as a penetration enhancer, while also producing a cooling effect on the skin as it evaporates. The nonvolatile solvent portion of the buffered solvent system is selected from lower alkylene glycols and lower glycol polymers. In certain embodiments, propylene glycol is used. The nonvolatile solvent slows the evaporation of the volatile solvent and reduces the vapor pressure of the buffered solvent system. The amount of this nonvolatile solvent component, as with the volatile solvent, is determined by the pharmaceutical compound or drug being used. When too little of the nonvolatile solvent is in the system, the pharmaceutical compound may crystallize due to evaporation of volatile solvent, while an excess may result in a lack of bioavailability due to poor release of drug from solvent mixture. The buffer component of the buffered solvent system may be selected from any buffer commonly used in the art; in certain embodiments, water is used. A common ratio of ingredients is about 20% of the nonvolatile solvent, about 40% of the volatile solvent, and about 40% water. There are several optional ingredients which can be added to the topical composition. These include, but are not limited to, chelators and gelling agents. Appropriate gelling agents can include, but are not limited to, semisynthetic cellulose derivatives (such as hydroxypropylmethylcellulose), synthetic polymers, galactomannan polymers (such as guar and derivatives thereof) and cosmetic agents.

Lotions include those suitable for application to the skin or eye. An eye lotion may comprise a sterile aqueous solution optionally containing a bactericide and may be prepared by methods similar to those for the preparation of drops. Lotions or liniments for application to the skin may also include an agent to hasten drying and to cool the skin, such as an alcohol or acetone, and/or a moisturizer such as glycerol or an oil such as castor oil or arachis oil.

Creams, ointments or pastes are semi-solid formulations of the active ingredient for external application. They may be made by mixing the active ingredient in finely-divided or powdered form, alone or in solution or suspension in an aqueous or non-aqueous fluid, with the aid of suitable machinery, with a greasy or non-greasy base. The base may comprise hydrocarbons such as hard, soft or liquid paraffin, glycerol, beeswax, a metallic soap; a mucilage; an oil of natural origin such as almond, corn, arachis, castor or olive oil; wool fat or its derivatives or a fatty acid such as steric or oleic acid together with an alcohol such as propylene glycol or a macrogel. The formulation may incorporate any suitable surface active agent such as an anionic, cationic or non-ionic surfactant such as a sorbitan ester or a polyoxyethylene derivative thereof. Suspending agents such as natural gums, cellulose derivatives or inorganic materials such as silicaceous silicas, and other ingredients such as lanolin, may also be included.

Drops may comprise sterile aqueous or oily solutions or suspensions and may be prepared by dissolving the active ingredient in a suitable aqueous solution of a bactericidal and/or fungicidal agent and/or any other suitable preservative, and, in certain embodiments, including a surface active agent. The resulting solution may then be clarified by filtration, transferred to a suitable container which is then sealed and sterilized by autoclaving or maintaining at 98° C.-100° C. for half an hour. Alternatively, the solution may be sterilized by filtration and transferred to the container by an aseptic technique. Examples of bactericidal and fungicidal agents suitable for inclusion in the drops are phenylmercuric nitrate or acetate (0.002%), benzalkonium chloride (0.01%) and chlorhexidine acetate (0.01%). Suitable solvents for the preparation of an oily solution include glycerol, diluted alcohol and propylene glycol. Formulations for topical administration in the mouth, for example buccally or sublingually, include lozenges comprising the active ingredient in a flavored basis such as sucrose and acacia or tragacanth, and pastilles comprising the active ingredient in a basis such as gelatin and glycerin or sucrose and acacia.

For administration by inhalation, compounds may be conveniently delivered from an insufflator, nebulizer pressurized packs or other convenient means of delivering an aerosol spray. Pressurized packs may comprise a suitable propellant such as dichlorodifluoromethane, trichlorofluoromethane, dichlorotetrafluoroethane, carbon dioxide or other suitable gas. In the case of a pressurized aerosol, the dosage unit may be determined by providing a valve to deliver a metered amount. Alternatively, for administration by inhalation or insufflation, the compounds according to the invention may take the form of a dry powder composition, for example a powder mix of the compound and a suitable powder base such as lactose or starch. The powder composition may be presented in unit dosage form, in for example, capsules, cartridges, gelatin or blister packs from which the powder may be administered with the aid of an inhalator or insufflator.

In some cases, a Formula (I) formulation is provided with one or more additional therapeutic ingredients. By way of example only, if one of the side effects experienced by a patient upon receiving one of the compounds herein is hypertension, then it may be appropriate to administer an anti-hypertensive agent in combination with the initial therapeutic agent. Or, by way of example only, the therapeutic effectiveness of one of the compounds described herein may be enhanced by administration of an adjuvant (i.e., by itself the adjuvant may only have minimal therapeutic benefit, but in combination with another therapeutic agent, the overall therapeutic benefit to the patient is enhanced). Or, by way of example only, the benefit experienced by a patient may be increased by administering one of the compounds described herein with another therapeutic agent (which also includes a therapeutic regimen) that also has therapeutic benefit. By way of example only, in a treatment for diabetic neuropathic pain involving administration of one of the compounds described herein, increased therapeutic benefit may result by also providing the patient with another therapeutic agent for diabetes. In any case, regardless of the disease, disorder or condition being treated, the overall benefit experienced by the patient may simply be additive of the two therapeutic agents or the patient may experience a synergistic benefit. The multiple therapeutic agents (at least one of which is a compound disclosed herein) may be administered in any order or even simultaneously. If simultaneously, the multiple therapeutic agents may be provided in a single, unified form, or in multiple forms (by way of example only, either as a single pill or as two separate pills). One of the therapeutic agents may be given in multiple doses, or both may be given as multiple doses. If not simultaneous, the timing between the multiple doses may be any duration of time ranging from a few minutes to four weeks.

### Methods of Use

Formula (I) (e.g., a single polymorph or a mixture of Forms A-L) are efficacious as soluble epoxide hydrolase inhibitors. The present disclosure provides methods for inhibiting soluble epoxide hydrolase inhibitors with Formula (I). In many cases, such methods include the use of one or more of Forms A-L for inhibiting soluble epoxide hydrolase.

Among other uses, Formula (I) can be administered to mediate disorder or disease is one in which modulation of sEH results in some effect on the underlying condition or disease (e.g., a sEH inhibitor or antagonist results in some improvement in patient well-being in at least some patients). Such disorders and diseases may include seizure disorders, such as epilepsy, nephropathy, cardiomyopathy, hypertension, pain, inflammation, inflammatory pain, post-surgical pain, neuropathic pain, diabetic neuropathic pain, tissue wounds or pain therefrom, acute inflammation, inflammation from sepsis, pancreatitis, multiple trauma such as injury to the brain, and tissue injury, such as laceration of the musculature, brain surgery, hemorrhagic shock, and immune-mediated organ injuries, adult respiratory distress syndrome, emphysema, chronic bronchitis, obstructive pulmonary disease, chronic obstructive pulmonary disease (COPC), small airway disease, interstitial lung disease (ILD), idiopathic pulmonary fibrosis, burning or pain in dermatoses such as dermatitis, chemical burns, thermal burns, reddening of the skin, and chemically induced lesions, neuralgia, pain caused by trauma or irritation to peripheral nerves near the surface of the skin.

In many cases, Formula (I) inhibits sEH without or while negligibly inhibiting microsomal epoxide hydrolase (hereinafter “mEH”), which is important for sodium transport and xenobiotic degradation. The advantageous solubilities of Forms A-L can facilitate formulation for cytosol and peroxisomal delivery (in which sEH is typically localized), and minimally to mEH, which is primarily cell membrane-anchored.

In particular embodiments, a formulation of the present invention is administered once a day. However, the formulations may also be formulated for administration at any frequency of administration, including once a week, once every 5 days, once every 3 days, once every 2 days, twice a day, three times a day, four times a day, five times a day, six times a day, eight times a day, every hour, or any greater frequency. Such dosing frequency is also maintained for a varying duration of time depending on the therapeutic regimen. The duration of a particular therapeutic regimen may vary from one-time dosing to a regimen that extends for months or years. The formulations are administered at varying dosages, but typical dosages are one to two drops at each administration, or a comparable amount of a gel or other formulation. One of ordinary skill in the art would be familiar with determining a therapeutic regimen for a specific indication.

Formula (I) may be administered orally, topically, or via injection at a dose of from 0.1 to 500 mg/kg per day. The dose range for adult humans is generally from 5 mg to 2 g/day. Tablets or other forms of presentation provided in discrete units may conveniently contain an amount of one or more compounds which is effective at such dosage or as a multiple of the same, for instance, units containing 5 mg to 500 mg, usually around 10 mg to 200 mg.

### EXAMPLES

The following examples are provided to further illustrate the embodiments of the present invention, but are not intended to limit the scope of the invention. While they are typical of those that might be used, other procedures, methodologies, or techniques known to those skilled in the art may alternatively be used.

### Experimental Methods

### Differential Scanning Calorimetry (DSC)

Differential scanning calorimetry analyses were performed on each sample “as is.” Unless otherwise specified, samples were weighed in an aluminum pan, covered with a pierced lid, and then crimped and analyzed from 30-300° C. ramped at 10° C./min.

### Thermal Gravimetric Analysis (TGA)

Thermal gravimetric analyses were performed on each sample “as is.” Unless otherwise noted, samples were weighed in an alumina crucible and analyzed from 30-350° C. at 10° C./min.

### X-Ray Powder Diffraction (XRD)

X-ray powder diffraction was performed on each sample “as is.” Samples were placed on Si zero-return ultra-micro sample holders, and analyses were performed using a 10 mm irradiated width and the parameters outlined in TABLE 1. Following analysis, the data were converted from adjustable to fixed slits using the 'Pert HighScore Plus software using a fixed divergence slit size of 1.00°, 1.59 mm and a crossover point of 44.3°Omega.

TABLE 1

| X-ray tube: | Cu KEICu K K K ube: |
|---|---|
| Detector: | X'Celerator |
| ASS Primary Slit: | Fixed 1° |
| Divergence Slit (Prog): | Automatic - 5 mm irradiated length |
| Soller Slits: | 0.02 radian |
| Scatter Slit (PASS): | Automatic - 5 mm observed length |
| Scan Range: | 3.0°-45.0° 0 |
| Scan Mode: | Continuous |
| Step Size: | 0.02° |
| Time per Step: | 10 s |
| Active Length: | 2.54° |

### Moisture-Sorption Analysis (DVS)

Gravimetric moisture sorption experiment was performed on all materials by first holding the sample at 40% RH and 25° C. until an equilibrium weight was reached or for a maximum of 4 h. The sample was then subjected to an isothermal (25° C.) adsorption scan from 40% to 90% R H in steps of 10% RH. The sample was allowed to equilibrate to an asymptotic weight at each point for a maximum of 4 h. Following adsorption, a desorption scan from 90% to 0% RH (at 25° C.) was run in steps of −10% R H allowing a maximum of 4 h for equilibration to an asymptotic weight. An adsorption scan was then performed from 0% RH to 40% RH in steps of +10% R H. The sample was then dried for more than 2 h at 60° C. 0% R H and the resulting solid analyzed by XRD.

### Nuclear Magnetic Resonance

Sample was dissolved in DMSO-d6. 1H NMR spectra were acquired at 300 MHz or 500 MHz using 5 mm broadband (1H-X) Z gradient probe. A 30 degree pulse with 20 ppm spectral width, 1.0 s repetition rate, and 32 transients were utilized in acquiring the spectra.

### Polarized Optical Microscopy

Samples were examined using a polarized light microscope combined with a digital camera (1600×1200 resolution). A small amount of sample was dispersed on glass slide with minimum mineral oil and 100× or 200× magnification. Parameters for the polarized optical microscopy measurements are provided in TABLE 2. A calibration curve at 254 nm is provided in FIG. 38.

TABLE 2

| System: | Agilent 1100 Series HPLC | |
|---|---|---|
| Column: | Y M C-Pack Pro C18; 150 × 3.0 mm I.D. S-3 μm. 12 nm | |
| Column Temperature: | Ambient | |
| Auto Sampler Temp: | Ambient | |
| Flow Rate: | 0.5 | mL/min |
| Injection Volume: | 2 | μL |
| Run Time: | 20 | minutes |
| Detection | 254 | nm |

| | |
|---|---|
| Mobile Phases | A - Water + 0.1% formic acid |
| | B - Acetonitrile + 0.1% formic acid |
| Diluent | Acetonitrile |
| Gradient | Time = 0 min.: 95% mobile phase A, 5% mobile phase B |
| | Time = 14 min.: 5% mobile phase A, 95% mobile phase B |
| | Time = 18 min.: 5% mobile phase A, 95% mobile phase B |
| | Time = 20 min.: 95% mobile phase A, 5% mobile phase B |

### Example 1

### Synthesis of Tert-Butyl 4-(3-(3-fluoro-4-(trifluoromethoxy)phenyl)ureido)piperidine-1-carboxylate (Compound S1)

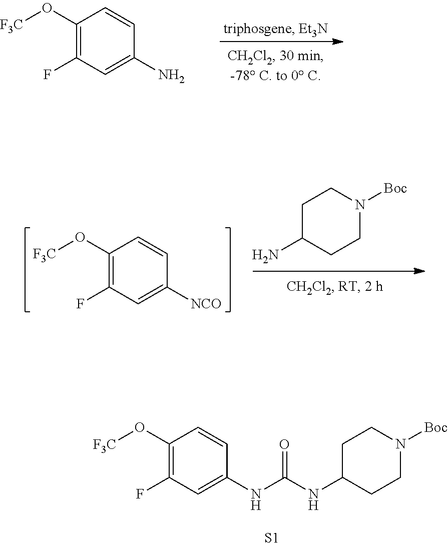

meta-Fluoro-4-(trifluoromethoxy)aniline (500 mg, 2.56 mmol) and triethylamine (388 mg, 3.84 mmol) were dissolved in CH₂Cl₂ (4 mL) and added dropwisely into a solution of triphosgene (341 mg, 1.15 mmol) dissolved in CH₂Cl₂ (5 mL) at −78° C. The reaction mixture was stirred at 0° C. for 1 hour and was then cooled to −78° C. 4-amino-1-Boc-piperidine (769 mg, 3.84 mmol) and triethylamine (388 mg, 3.84 mmol) were dissolved in CH₂Cl₂ (4 mL) and the suspension was added dropwisely to the reaction mixture at −78° C. The reaction mixture was stirred at room temperature (RT) for 2 hours. The reaction was quenched by addition of water. The organic layer was isolated and the organic layer was further washed by HCl solution (1M) 4 times. The organic layer was dried over anhydrous magnesium sulfate and concentrated in vacuo yielding final crude product (1.05 g, 86% pure, 2.13 mmol, 83.4% yield).

The impurities (which included 1,3-bis(3-fluoro-4-(trifluoromethoxy)phenyl)urea) were removed by column chromatography using EtOAc:Hex (1:1).

1H NMR (dg-DMSO, 300 Mhz): A: d 8.77 (s, 1H), 7.66 (dd, J=13.5, 2.4 Hz, 1H), 7.38 (t, J=8.1 Hz, 1H), 7.10 (d, J=9 Hz, 1H), 6.33 (d, J=7.5 Hz, 1H), 3.81 (d, J=12.9 Hz, 2H), 3.6-3.8 (m, 1H), 2.8-3.0 (m, 2H), 1.78 (dd, J=12.3 Hz, 3.3 Hz, 2H), 1.40 (s, 9H), 1.2-1.4 (m, 2H); 38: d 9.28 (s, 1H), 7.69 (dd, J=14.9, 2.4 Hz, 1H), 7.46 (t, J=9 Hz, 1H), 7.2-7.3 (m, 1H).

### Example 2

### Synthesis of 1-(3-fluoro-4-(trifluoromethoxy)phenyl)-3-(piperidin-4-yl)urea (Compound S2)

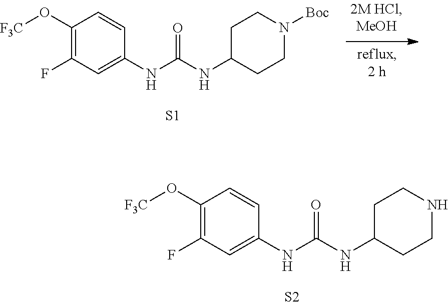 [SMILES: CC(C)(C)OC(=O)N1CCC(NC(=O)Nc2ccc(OC(F)(F)F)c(F)c2)CC1 ; "S1" ; O=C(Nc1ccc(OC(F)(F)F)c(F)c1)NC1CCNCC1 ; "S2"]

Compound S1 (186 mM) was dissolved in HCl solution (2M, MeOH) to make reaction mixture. The resulting solution was refluxed for 2 hours. The solvent was removed under vacuo and the crude reaction product was adjusted to pH 12 with NaOH. The precipitate product S2 was filtered and dried under high vacuum.

### Example 3

### Synthesis of (S)-1-(3-fluoro-4-(trifluoromethoxy)phenyl)-3-(1-(2-methylbutanoyl)piperidin-4-yl)urea (Formula (I))

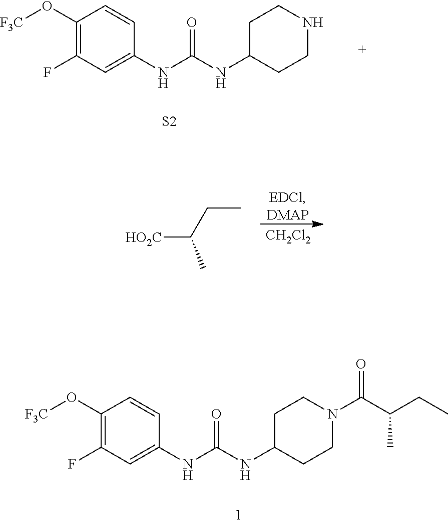

(S)-2-methylbutanoic acid (14 mg, 140 μmol) was activated with molar equivalents of 1-ethyl-3-(3-dimethylaminopropyl) carbodiimide (EDCl) and 4-dimethylaminopyridine (DMAP) in CH₂CL₂ and then combined with Compound S2 (30 mg, 93.4 μmol). The reaction was stirred for 12 hours at room temperature, and then quenched by addition of 1 M HCl. The organic layer was collected and the aqueous layer was extracted 4 times with EtOAc. The organic layer and EtOAc were combined and dried over anhydrous magnesium sulfate, and then concentrated in vacuo. The product was purified by flash chromatography and eluted by ethyl acetate. The collected fraction was dried in vacuo, yielding white solid Formula (I). The product was further purified by recrystallization using methanol and water (30 mg, 83.9 μmol, 79.2% yield, Purity (H-NMR): >95%).

M P: 147-147.8° C. 1H NMR (de-DMSO, 600 Mhz): d 8.78 (d, J=17.4 Hz, 1H), 7.66 (dd, J=13.8 Hz, 2.4 Hz, 1H), 7.39 (t, J=9 Hz, 1H), 7.11 (dd, J=9 Hz, 1.2 Hz, 1H), 6.3-6.4 (m, 1H), 4.23 (t, J=13.2 Hz, 1H), 3.88 (d, J=10.8 Hz, 1H), 3.6-3.8 (m, 1H), 3.1-3.2 (m, 1H), 2.6-2.8 (m, 2H), 1.8-1.9 (br, 1H), 1.7-1.8 (m, 1H), 1.5-1.6 (m, 1H), 1.1-1.4 (m, 3H), 0.9-1.0 (m, 3H), 0.7-0.9 (m, 3H).

### Example 4

### Assessment of Multiple Crystallization Conditions

This example covers crystallization of Formula (I) in multiple solvent systems. Solvents were selected to span a range of polarities, functionalities, and classifications according to the International Conference on Harmonization (ICH) with preferences given to class II and class III solvents. The solvents also varied in terms of Formula (I) solubility. For these analyses, approximately 4-6 mg of Formula (I) was dispensed into a 7 mL glass vials and selected solvents were added in 100 μL of aliquots to completed is solution at room temperature. After each addition of solvent, the vials were shaken and visually inspected for residual solids. If required, heating to 50° C. or heated to reflux (for low boiling point solvents) for approximately two minutes was performed to ensure complete dissolution. If residual solids remained, additional solvent aliquots were added. The solvent addition was discontinued when complete dissolution was achieved.

TABLE 3 summarizes the solvents used for crystallization, and corresponding solubility of Formula (I) at room temperature (RT) and 50° C. Based on these solubility data, sixteen solvents were selected as primary solvents: methanol (MeOH), ethanol (EtOH), isopropanol (IPA), n-BuOH, acetonitrile (ACN), tetrahydrofuran (THF), 2-methyltetrahydrofuran (2-MeTHF), ethyl acetate (EtOAc), isopropyl acetate (IPAc), acetone, 2-butanone (methyl ethyl ketone, ME EK), methyl isobutyl ketone (MIBK), dichloromethane (DCM), toluene, methyl tert-butyl ether (MTBE), and dioxane. Three solvents, water, n-heptane, and c-hexane, were selected as antisolvents for crystallization. A total of twelve unique XRD patterns, as provided in FIG. 1 Panels A-L were identified from single-solvent and binary-solvent crystallizations.

TABLE 3
SUMMARY OF SOLUBILITY SCREENING OF FORMULA (I)

| | | | | | | Nominal | Solubility | |
|---|---|---|---|---|---|---|---|---|
| Formula (I) | | bp | Temp | | | Solubility | Range | Solvent ICH |
| (mg) | Solvent | (° C.) | (° C.) | Vol (mL) | Soluble? | (mg/mL ) | (mg/mL ) | Class |
| 5.44 | Water | 100 | 50 | 4.0 | No | <1.36 | <1.36 | * |
| 4.32 | MeOH | 64.7 | RT | 0.1 | Yes | 43.2 | ≥43.2 | 2 |
| 4.12 | EtOH | 78 | RT | 0.1 | Yes | 41.2 | ≥41.2 | 3 |
| 5.60 | IPA | 82 | RT | 0.1 | Yes | 56.0 | ≥56.0 | 3 |
| 4.26 | n-BuOH | 118 | RT | 0.1 | Yes | 42.6 | ≥42.6 | 3 |
| 5.33 | ACN | 81 | RT | 0.1 | Yes | 48.3 | ≥48.3 | 2 |
| 4.80 | THF | 66 | RT | 0.1 | Yes | 48.0 | ≥48.0 | 2 |
| 5.04 | 2-MeTHF | 78 | RT | 0.2 | Yes | 25.2 | 25.2-50.4 | * |
| 5.43 | EtOAc | 77 | RT | 0.1 | Yes | 54.3 | ≥54.3 | 3 |
| 4.67 | IPAC | 89 | RT | 0.2 | Yes | 23.4 | 23.4-46.7 | 3 |
| 4.37 | Acetone | 56 | RT | 0.1 | Yes | 43.7 | ≥43.7 | 3 |
| 5.04 | MEK | 80 | RT | 0.1 | Yes | 50.4 | ≥50.4 | 3 |
| 5.30 | MIBK | 117 | RT | 0.1 | Yes | 48.0 | ≥48.0 | 3 |
| 4.66 | DCM | 40 | RT | 0.1 | Yes | 46.6 | ≥46.6 | 2 |
| 5.00 | Toluene | 110 | 50 | 0.5 | Yes | 10.0 | 10.0-25.0 | 2 |
| 5.68 | MTBE | 56 | 50 | 1.0 | Yes | 5.68 | 5.68-11.4 | 3 |
| 5.28 | n-Heptane | 98 | 50 | 4.0 | No | <1.32 | <1.32 | 3 |
| 4.67 | c-Hexane | 69 | 50 | 4.0 | No | <1.17 | <1.17 | 2 |
| 4.78 | DMSO | 189 | RT | 0.1 | Yes | 47.8 | ≥47.8 | 3 |
| 4.61 | DMF | 148 | RT | 0.1 | Yes | 46.1 | ≥46.1 | 2 |
| 5.52 | NMP | 202 | RT | 0.1 | Yes | 55.2 | ≥55.2 | 3 |
| 5.22 | n-Propanol | 207 | RT | 0.1 | Yes | 52.2 | ≥52.2 | 3 |
| 4.68 | Dioxane | 214 | RT | 0.5 | Yes | 9.36 | 9.36-23.4 | 2 |

Formula (I) crystallization was first analyzed in single-solvent systems lacking antisolvent. For these analyses, approximately 30-70 mg of Formula (I) was weighed into 7 mL clear glass vials equipped with stir bars and dissolved with a minimum amount of solvent required for dissolution (minimum 0.1 mL). Fast cooling was performed by placing the vials in a 4° C. refrigerator for 24 hours, while slow cooling was performed by decreasing the temperatures of the vials by 20° C./h with stirring. The resulting solids were isolated by centrifuge filtration through 0.45 m centrifuge filters. When solids were not produced, the solution was evaporated under nitrogen as an additional attempt to obtain solid. All obtained solids were analyzed by XRD to determine the solid pattern. For each recrystallization, Formula (I) was dissolved in solvent and recrystallized from either 40° C., 50° C., or 70° C., based on the boiling point of the primary solvent. In total, eight unique XRD patterns were observed for the crystals generated from the crystallizations, the results of which are summarized in TABLE 4 for the slow and fast cooling experiments, respectively.

TABLE 4

| For- | | | | | | |
|---|---|---|---|---|---|---|
| mula | | | | | | |
| (I) | | Vol- | | | | |
| FORM | | ume | Temp | | Precipitation/ | |
| A (mg) | Solvent | (mL) | (° C.) | Cooling | Isolation | XRD |
| 39.00 | MeOH | 0.1 | 50 | Slow (20° C./h) | Clear/evaporate | B |
| 35.83 | EtOH | 0.1 | 70 | Slow (20° C./h) | Clear/evaporate | B |
| 30.94 | IPA | 0.1 | 70 | Slow (20° C./h) | Clear/evaporate | B |
| 32.89 | n-BuOH | 0.1 | 70 | Slow (20° C./h) | Clear/evaporate | B |
| 36.72 | ACN | 0.2 | 70 | Slow (20° C./h) | ppt/evaporate | A + |
| | | | | | | B |
| 30.20 | THF | 0.1 | 50 | Slow (20° C./h) | Clear/evaporate | G |
| 45.91 | 2-MeTHF | 0.2 | 70 | Slow (20° C./h) | ppt/evaporate | H |
| 72.60 | EtOAc | 0.1 | 70 | Slow (20° C./h) | ppt/evaporate | B + |
| | | | | | | E |
| 30.96 | IPAC | 0.1 | 70 | Slow (20° C./h) | ppt/evaporate | H |
| 33.13 | Acetone | 0.1 | 50 | Slow (20° C./h) | Clear/evaporate | B + |
| | | | | | | E |
| 30.25 | MEK | 0.1 | 70 | Slow (20° C./h) | Clear/evaporate | G |
| 30.94 | MIBK | 0.1 | 70 | Slow (20° C./h) | Clear/evaporate | H* |
| 29.56 | DCM | 0.1 | 40 | Slow (20° C./h) | Clear/evaporate | I |
| 32.28 | Toluene | 1.4 | 70 | Slow (20° C./h) | ppt/evaporate | J |
| 35.37 | MTBE | 4.6 | 50 | Slow (20° C./h) | ppt/evaporate | K |
| 39.77 | Dioxane | 0.1 | 70 | Slow (20° C./h) | ppt/evaporate | G |
| 40.00 | MeOH | 0.1 | 50 | Fast (4° C.) | Clear/evaporate | A |
| 33.53 | EtOH | 0.1 | 70 | Fast (4° C.) | Clear/evaporate | B |
| 30.35 | IPA | 0.1 | 70 | Fast (4° C.) | Clear/evaporate | B |
| 34.73 | n-BuOH | 0.1 | 70 | Fast (4° C.) | Clear/evaporate | B* |
| 31.65 | ACN | 0.2 | 70 | Fast (4° C.) | ppt/evaporate | I |
| 33.20 | THF | 0.1 | 50 | Fast (4° C.) | Clear/evaporate | G |
| 36.54 | 2-MeTHF | 0.2 | 70 | Fast (4° C.) | ppt/evaporate | H |
| 29.37 | EtOAc | 0.1 | 70 | Fast (4° C.) | ppt/evaporate | G + |
| | | | | | | I |
| 31.74 | IPAC | 0.1 | 70 | Fast (4° C.) | ppt/evaporate | H |
| 33.83 | Acetone | 0.1 | 50 | Fast (4° C.) | Clear/evaporate | E + |
| | | | | | | I |
| 29.12 | MEK | 0.1 | 70 | Fast (4° C.) | Clear/evaporate | G + |
| | | | | | | I |
| 36.67 | MIBK | 0.1 | 70 | Fast (4° C.) | Clear/evaporate | na* |
| 38.33 | DCM | 0.1 | 40 | Fast (4° C.) | Clear/evaporate | I |
| 34.33 | Toluene | 1.4 | 70 | Fast (4° C.) | ppt/evaporate | A |
| 31.29 | MTBE | 4.6 | 50 | Fast (4° C.) | ppt/evaporate | K |
| 32.40 | Dioxane | 0.1 | 70 | Fast (4° C.) | ppt/evaporate | G |

Formula (I) recrystallization was then tested with binary-solvent systems containing water, n-heptane, or c-hexane as antisolvent. For these analyses, approximately 30-40 mg of Formula (I) was weighed into 7 mL clear glass vials equipped with stir bars and dissolved with a minimum amount of each primary solvent necessary for dissolution (minimum 0.1 mL). Anti solvents were added drop-wise at the same temperature until precipitate was observed or the vial volume was reached (˜7 mL). The vials were quickly cooled to 4° C. or slowly cooled by 20° C./hour as in the single-solvent recrystallizations. The resulting solids were isolated by centrifuge filtration. The samples without precipitation were evaporated to dryness under a gentle stream of nitrogen gas as an additional attempt to obtain solid. All obtained solids were analyzed by XRD to determine the solid form. The selection of primary solvents was based on high solubility of Formula (I) and miscibility with the antisolvent. For each binary-solvent system, Formula (I) was recrystallized from either 50° C. or 70° C. Ten unique XRD patterns were identified from the resultant crystals, and are summarized in TABLE 5-7 below.

TABLE 5

| Formula (I) | Primary | | | | | |
|---|---|---|---|---|---|---|
| Form C | Solvent | Antisolvent | Temp | | Precipitation/ | Formula (I) |
| Input (mg) | (Vol., ml) | (Vol., ml) | (° C.) | Cooling | Isolation | Product Form |
| 30.34 | MeOH (0.1) | Water (0.05) | 50 | Fast | clear/evaporate | F |
| | | | | (to 4° C.) | | |
| 31.76 | EtOH (0.1) | Water (0.1) | 70 | Fast | clear/evaporate | F |
| 30.29 | ACN (0.2) | Water (0.25) | 70 | Fast | ppt/ filtered | F |
| 35.45 | IPA (0.1) | Water (0.1) | 70 | Fast | clear/evaporate | F |
| 34.49 | THF (0.1) | Water (0.05) | 50 | Fast | clear/evaporate | F |
| 31.70 | A cetone (0.1) | Water (0.05) | 50 | Fast | clear/evaporate | F |
| 30.75 | DMSO (0.1) | Water (0.05) | 70 | Fast | ppt/filtered | F |
| 32.93 | DMF (0.1) | Water (0.1) | 70 | Fast | ppt/filtered | F |
| 37.55 | NMP (0.1) | Water (0.1) | 70 | Fast | ppt/filtered | F |
| 36.97 | n-Propanol (0.1) | Water (0.1) | 70 | Fast | clear/evaporate | B |
| 31.54 | Dioxane (0.1) | Water (0.1) | 70 | Fast | ppt/filtered | G |
| 33.89 | MeOH (0.1) | Water (0.05) | 50 | Slow | clear/evaporate | C |
| | | | | (20° C./h) | | |
| 31.89 | EtOH (0.1) | Water (0.1) | 70 | Slow | clear/evaporate | F |
| 31.77 | ACN (0.1) | Water (0.25) | 70 | Slow | clear/evaporate | A |
| 31.92 | IPA (0.2) | Water (0.1) | 70 | Slow | clear/evaporate | D |
| 32.07 | THF (0.1) | Water (0.05) | 50 | Slow | clear/evaporate | F |
| 32.24 | Acetone (0.1) | Water (0.1) | 50 | Slow | clear/evaporate | A |
| 31.30 | DMSO (0.1) | Water (0.05) | 70 | Slow | ppt/filtered | D + F |
| 34.54 | DMF (0.1) | Water (0.1) | 70 | Slow | ppt/filtered | D |
| 37.04 | NMP (0.1) | Water (0.1) | 70 | Slow | ppt/filtered | F |
| 33.54 | n-Propanol (0.1) | Water (0.1) | 70 | Slow | clear/evaporate | F |
| 33.84 | Dioxane (0.1) | Water (0.1) | 70 | Slow | ppt/filtered | G |

TABLE 6

| Formula (I) | | | | | | Formula (I) |
|---|---|---|---|---|---|---|
| Form C | Primary Solvent | Antisolvent | Temp | | Precipitation/ | Product |
| Input (mg) | (Vol., ml) | (Vol., ml) | (° C.) | Cooling | Isolation | Form |
| 30.01 | MeOH (0.1) | n-Heptane (8) | 50 | Fast | clear/evaporate | E |
| | | | | (to 4° C.) | | |
| 31.41 | EtOH (0.1) | n-Heptane (1.4) | 70 | Fast | ppt/filtered | E |
| 34.72 | ACN (0.2) | n-Heptane (7.5) | 70 | Fast | ppt/filtered | E |
| 31.96 | IPA (0.15) | n-Heptane (2) | 70 | Fast | ppt/filtered | E |
| 31.76 | THF (0.1) | n-Heptane (0.2) | 50 | Fast | ppt/evaporate | G |
| 33.11 | Acetone (0.1) | n-Heptane (0.4) | 50 | Fast | ppt/filtered | E |
| 37.07 | DMSO (0.1) | n-Heptane (7.2) | 70 | Fast | clear/evaporate | D |
| 34.70 | DMF (0.1) | n-Heptane (7) | 70 | Fast | clear/evaporate | F |
| 35.14 | NMP (0.1) | n-Heptane (7) | 70 | Fast | clear/evaporate | D |
| 35.86 | n-Propanol (0.1) | n-Heptane (2.5) | 70 | Fast | ppt/filtered | E |
| 31.98 | Dioxane (0.25) | n-Heptane (2.5) | 70 | Fast | ppt/filtered | G |
| 35.73 | MeOH (0.1) | n-Heptane (8) | 50 | Slow | clear/evaporate | E |
| | | | | (20° C./h) | | |
| 30.22 | EtOH (0.1) | n-Heptane (3.2) | 70 | Slow | ppt/filtered | E |
| 33.67 | ACN (0.1) | n-Heptane (7.5) | 70 | Slow | ppt/filtered | E |
| 34.90 | IPA (0.2) | n-Heptane (1.5) | 70 | Slow | ppt/filtered | E |
| 34.05 | THF (0.1) | n-Heptane (0.2) | 50 | Slow | ppt/evaporate | L |
| 33.68 | Acetone (0.1) | n-Heptane (0.4) | 50 | Slow | ppt/evaporate | B + E |
| 37.89 | DMSO (0.1) | n-Heptane (7) | 70 | Slow | clear/evaporate | F |
| 37.60 | DMF (0.1) | n-Heptane (7) | 70 | Slow | clear/evaporate | D |
| 30.16 | NMP (0.1) | n-Heptane (7) | 70 | Slow | clear/evaporate | F |
| 34.34 | n-Propanol (0.1) | n-Heptane (2.5) | 70 | Slow | ppt/filtered | E |
| 32.86 | Dioxane (0.25) | n-Heptane (2.5) | 70 | Slow | ppt/evaporate | G |

TABLE 7

| Formula (I) | | | | | | Formula (I) |
|---|---|---|---|---|---|---|
| Form C | Primary Solvent | Antisolvent | T emp | | Precipitation/ | Product |
| Input (mg) | (Vol., ml) | (Vol., ml) | (° C.) | Cooling | Isolation | Form |
| 33.62 | MeOH (0.1) | c-Hexane (6.6) | 50 | Fast | clear/evaporate | F |
| | | | | (to 4° C.) | | |
| 34.12 | EtOH (0.1) | c-Hexane (6) | 70 | Fast | clear/evaporate | F |
| 40.26 | ACN (0.2) | c-Hexane (6) | 70 | Fast | ppt/filtered | H |
| 34.54 | IPA (0.1) | c-Hexane (2) | 70 | Fast | ppt/filtered | H + F |
| 40.44 | THF (0.1) | c-Hexane (0.2) | 50 | Fast | ppt/evaporate | H |
| 42.24 | Acetone (0.1) | c-Hexane (0.4) | 50 | Fast | ppt/evaporate | G |
| 39.23 | DMSO (0.1) | c-Hexane (6) | 70 | Fast | clear/evaporate | D |
| 31.10 | DMF (0.1) | c-Hexane (6) | 70 | Fast | clear/evaporate | D |
| 38.01 | NMP (0.1) | c-Hexane (6) | 70 | Fast | clear/evaporate | F |
| 43.71 | n-Propanol (0.1) | c-Hexane (2) | 70 | Fast | ppt/filtered | F |
| 31.93 | Dioxane (0.45) | c-Hexane (0.9) | 70 | Fast | ppt/filtered | F |
| 39.28 | MeOH (0.1) | c-Hexane (6.6) | 50 | Slow | clear/evaporate | F |
| | | | | (20° C./h) | | |
| 30.67 | EtOH (0.1) | c-Hexane (6) | 70 | Slow | clear/ | F + H |
| 31.64 | ACN (0.2) | c-Hexane (6) | 70 | Slow | ppt/filtered | B |
| 31.34 | IPA (0.1) | c-Hexane (2) | 70 | Slow | ppt/filtered | F |
| 36.74 | THF (0.1) | c-Hexane (0.2) | 50 | Slow | ppt/evaporate | G |
| 33.29 | Acetone (0.1) | c-Hexane (0.4) | 50 | Slow | ppt/evaporate | H |
| 33.45 | DMSO (0.1) | c-Hexane (6) | 70 | Slow | clear/evaporate | D |
| 35.66 | DMF (0.1) | c-Hexane (6) | 70 | Slow | clear/evaporate | F |
| 32.94 | NMP (0.1) | c-Hexane (6) | 70 | Slow | clear/evaporate | D + F |
| 36.77 | n-Propanol (0.1) | c-Hexane (2) | 70 | Slow | ppt/filtered | F |
| 32.44 | Dioxane (0.45) | c-Hexane (0.9) | 70 | Slow | ppt/filtered | B |

### Example 5

### Polymorph Characterization

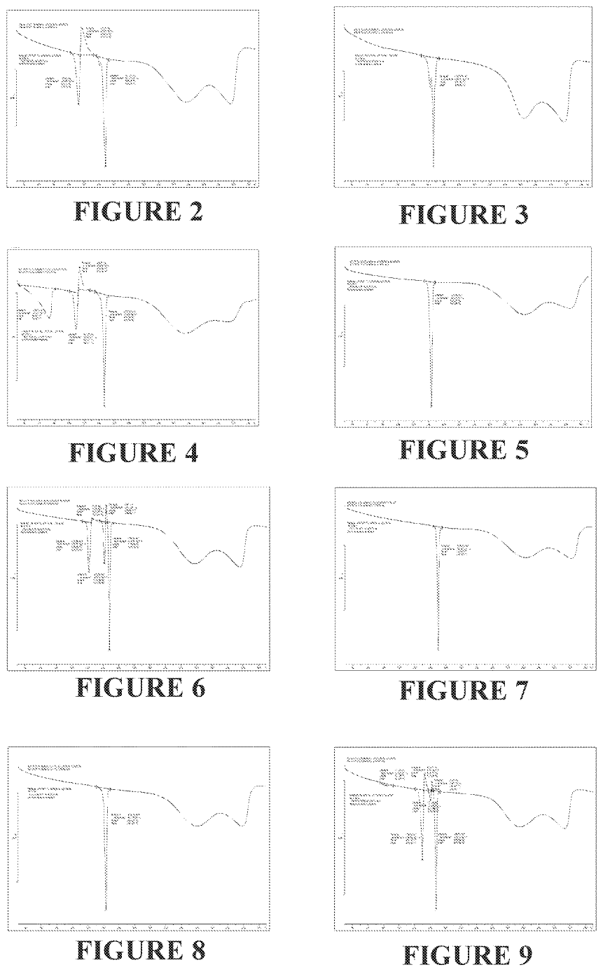

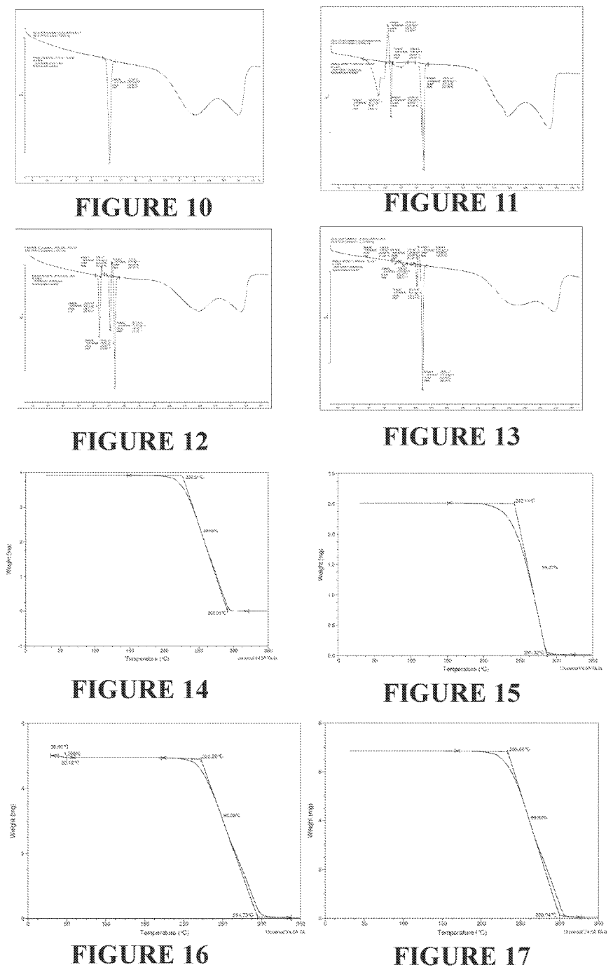

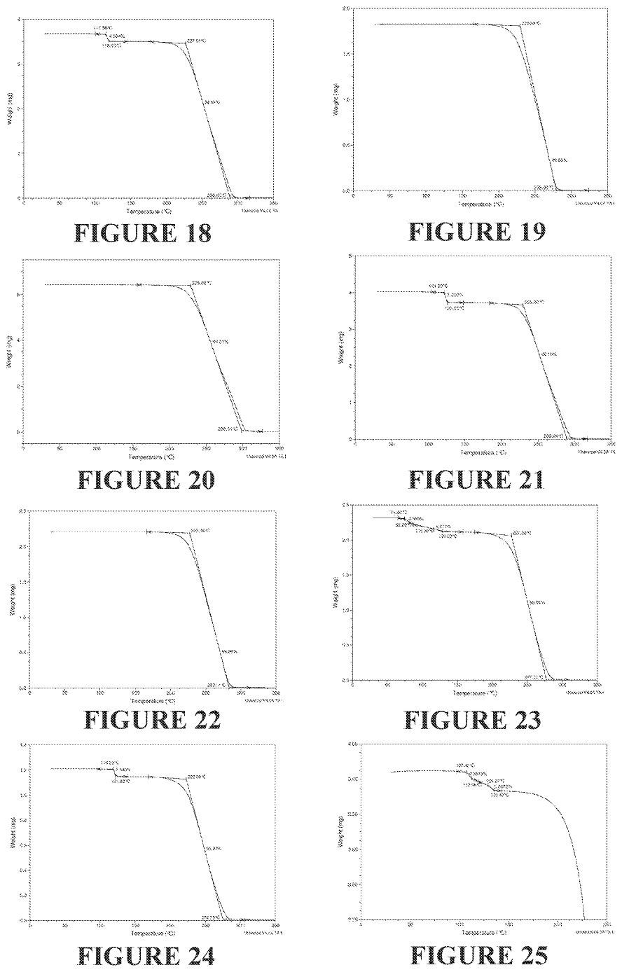

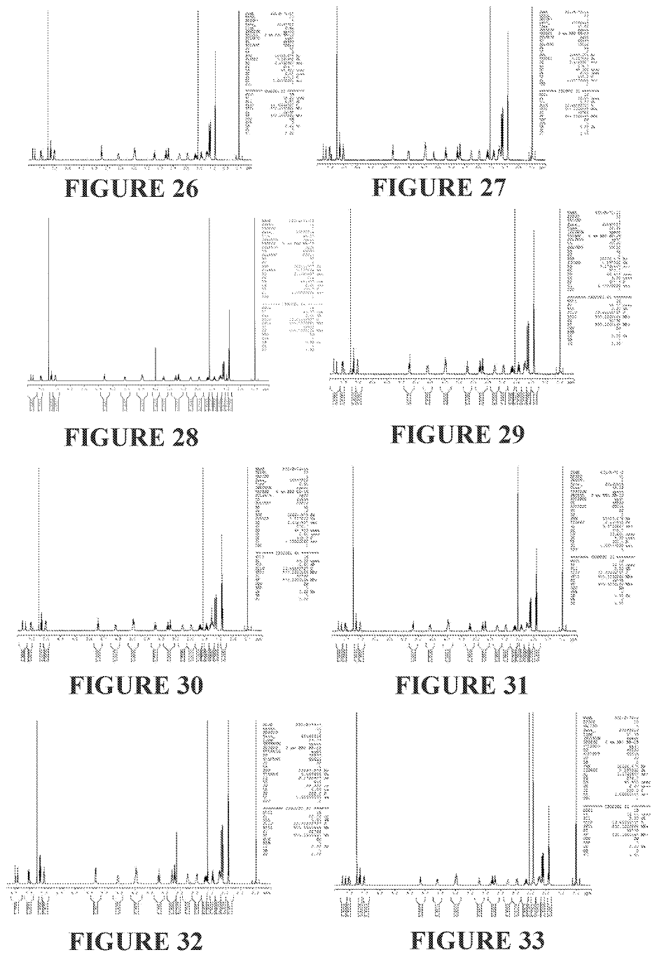

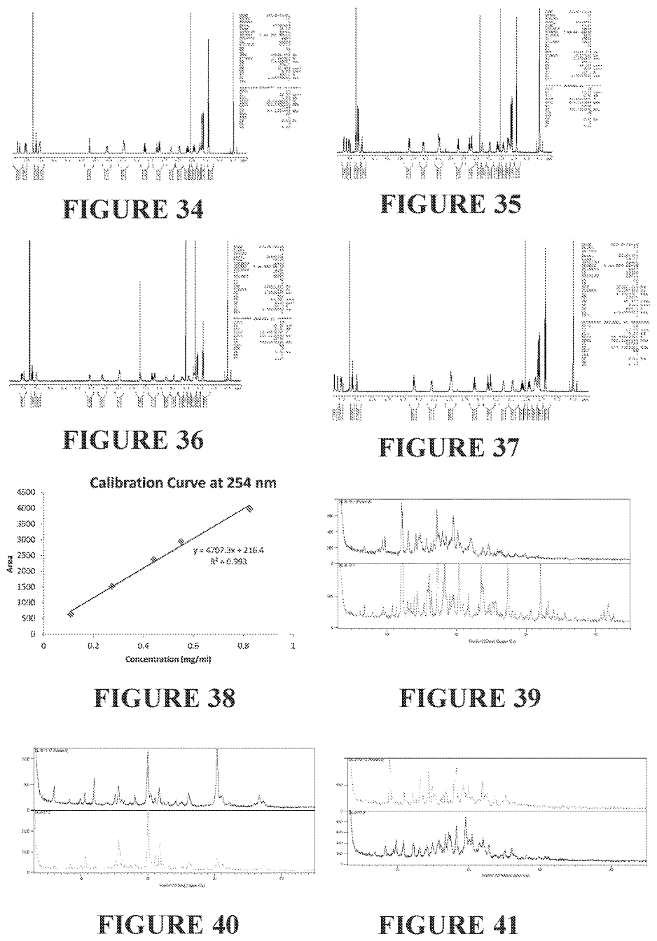

This example covers characterization of the twelve polymorphs identified in EXAMPLE 4. Each polymorph was analyzed with XRD (FIG. 1 Panels A-L), differential scanning calorimetry (FIGS. 2-13), thermogravimetric analysis (FIGS. 14-25) for polymorph and pseudopolymorph identification, and nuclear magnetic resonance (FIGS. 26-37) for confirmation of chemical integrity and residual solvent. Exemplary conditions for producing each of the twelve polymorphs and physical characterizations are summarized in TABLE 8.

DSC results for each of Forms A-L are provided in FIGS. 2-13, respectively. While each sample exemplified a single polymorph, some samples exhibited evidence of trace presences of additional forms. In particular, DSC indicated small amounts of Form D in some preparations of Forms A, B, E, G, H, K and L, as well as the absence of additional polymorphs in Forms D, F, and I. Hydrated Form C was not detected in any of the other polymorphs by DSC.

Results of the thermogravimetric analyses of Forms A-L are provided in FIGS. 14-25, respectively. All forms exhibited weight loss above 150° C., corresponding to Formula (I) desportion. Only Forms C, E, H, J, K, L exhibited weight changes below 150° C., indicating solvent loss. Form C weight loss at 39° C.-50° C. likely reflected methanol loss. Forms E, H, J, K, and L exhibited weight loss around 120° C., corresponding to organic solvent loss.

FIGS. 26-37 provide NM R spectra of Forms A-L, respectively. In addition to Formula (I), NMR identified about 2.5% methanol in Form C, about 0.9% isopropyl alcohol in Form E, about 0.04% acetone in Form G, about 7.1% c-hexane in Form H, about 8.8% toluene in Form J, about 8.8% MTBE in Form K, and about 0.2% TH F in Form L.

TABLE 8

| Crystallization | | | |
|---|---|---|---|
| Method | Pattern | DSC Thermal Events | TGA and NMR |
| Binary: acetone | A (anhydrate) | Endo: | TGA: |
| primary; water | | 113° C. melting of pattern A | No wt change below |
| antisolvent; | | 141° C. melting of pattern D (shoulder) | 150° C. |
| slow cooling | | 148° C. meting of patter F | NMR: |
| | | Exo: | No confirmed solvent |
| | | 117° C. crystallization of pattern | peak |
| | | F | |
| | | 127° C. crystallization of pattern D | |
| | | (shoulder) | |
| Binary: ACN | B (anhydrate) | Endo: | TGA: |
| primary; c- | | 141° C. melting of pattern D | No wt change below |
| hexane | | (shoulder) | 150° C. |
| antisolvent; | | 146° C. melting of pattern B | [symbol] |
| [symbol] | | | |
| Binary: MeOH | C (hydrate) | Endo: | TGA: |
| primary; water | | 73° C.: dehydration of bound water of | 1.4% wt change at |
| antisolvent; | | pattern C | range 39° C.-50° C. |
| slow cooling | | (Pattern C−> pattern A) | NMR: |
| | | 109° C. melting of pattern A | MeOH (estimated |
| | | 147° C. melting of pattern F | amount 2.5%) |
| | | Exo: | |
| | | 114° C. crystallization of pattern F | |
| Binary: IPA | D (anhydrate) | Endo: | TGA: |
| primary; water | | 143° C. melting of pattern D | No wt change below |
| antisolvent; | | | 150° C. |
| slow cooling | | | |
| Binary: IPA | E (anhydrate) | Endo: | TGA: |
| primary; n- | | 121° C. dehydration of bound water of | 4.7% wt change at |
| heptane | | pattern E | range 116° C.-119° C. |
| antisolvent; | | 142° C. melting of pattern D | NMR: |
| slow cooling | | 148° C. melting of pattern F | IPA (estimated amount |
| | | Exo: | 0.9%) |
| | | 126° C. crystallization of pattern D | |
| | | 144° C. crystallization of pattern F | |
| Binary: ACN | F | Endo: | TGA: |
| primary; water | (anhydrate) | 150° C. melting of pattern F | No wt change below |
| antisolvent; fast | | | 150° C. |
| cooling | | | NMR: |
| | | | No confirmed solvent |
| | | | peak |
| Binary: acetone | G | Endo: | TGA: |
| primary; c- | (anhydrate) | 143° C. melting of pattern G | No wt change below |
| hexane | | (Same melting point of pattern D | 150° C. |
| antisolvent; fast | | (143° C.), close to melting point of | NMR: |
| cooling | | pattern I (144° C.)) | Acetone (estimated |
| | | | amount 0.04%) |
| Binary: ACN | H (likely major | Endo: | TGA: |
| primary; c- | solvate pattern | 83° C.: dehydration of bound water of | No detectable wt |
| hexane | H with minor | pattern C | change at 83° C. |
| antisolvent; fast | hydrate | 129° C. desolvation of pattern H | 7.2% wt change at |
| cooling | pattern C) | 140° C. melting of pattern A | range 122° C.-126° C. |
| | | 148° C. melting of pattern F | NMR: |
| | | Exo: | c-hexane residual |
| | | 133° C. crystallization of pattern D | (estimated amount |
| | | 142° C. crystallization of patter F | 7.1%) |
| Single: ACN | I | Endo: | TGA: |
| primary fast | (anhydrate) | 144° C. melting of pattern I | No wt change below |
| cooling | | | 150° C. |
| | | | NMR: |
| | | | No confirmed solvent |
| | | | peak |
| Single: toluene | J | Endo: | TGA: |
| primary; slow | (solvate/ | 91° C. dehydration of bound | 3.7% wt change at |
| cooling | hydrate) | water/desolvation of pattern J | range 75° C.-82° C. |
| | (mixture?) | (major peak with a shoulder) | 4.6% wt change at |
| | | 140° C. melting of unknown | range 116° C.-129° C. |
| | | pattern | NMR: |
| | | 129° C. dehydration of bound | Toluene (estimated |
| | | water/desolvation of pattern J or | amount 8.8% ) |
| | | another unknown pattern | |
| | | 146° C. melting of pattern B | |
| | | 149° C. melting of pattern F | |
| | | Exo: | |
| | | 103° C. crystallization of unknown | |
| | | pattern | |
| Single: MTBE | K | Endo: | TGA: |
| primary; slow | (hydrate/ | 127° C. dehydration of bound water/ | 5.0% wt change at |
| cooling | solvate) | desolvation of pattern K | range 120° C.-122° C. |
| | | 142° C. melting of pattern D | NMR: |
| | | 148° C. melting of pattern F | MTBE (estimated |
| | | Exo: | amount 8.8%) |
| | | 130° C. crystallization of pattern D | |
| | | ° C. crystallization of pattern F | |
| Binary: THF | L | Endo: | TGA: |
| primary; n- | (hydrate/ | 114° C. dehydration of bound water/ | 0.4% wt change at |
| heptane | solvate, or | desolvation of pattern L | range 107° C.-113° C. |
| antisolvent; | mixture?) | 124° C. dehydration of bound water/ | 0.3% wt change at |
| slow cooling | | desolvation of pattern L | range 129° C.-135° C. |
| | | 141° C. melting of pattern D | NMR: |
| | | 148° C. melting of pattern F | THF (estimated amount |
| | | Exo: | 0.2%) |
| | | 138° C. crystallization of pattern D | |
| | | 143° C. crystallization of patter F | |

[symbol]

indicates data missing or illegible when filed

### Example 6

### Scaled-Up Methods for Polymorph Production

In order to produce sufficient quantities of each polymorph (e.g., Forms A, B, D, E, etc.) for further physical characterizations, Formula (I) was generated according to EXAMPLES 1-3, and then recrystallized using the solvent systems and conditions outlined in EXAMPLE 5. Parameters of the scaled-up recrystallizations are outlined in TABLE 9. Briefly, the scaled-up recrystallizations utilized between about 330 and 450 mg Formula (I), an about 10-fold greater amount than was used in the recrystallizations of EXAMPLE 4.

TABLE 9

| | Mass | | ANTI- | | | |
|---|---|---|---|---|---|---|
| | For- | SOL- | SOL- | | | |
| | mula | VENT | VENT | | | |
| XRD/ | (I) | (VOL- | (VOL- | TEMP. | | ISO- |
| FORM | (mg) | UME) | UME) | (° C.) | COOLING | LATION |
| FIG. | 352.1 | ACN | None | 70 | Fast | precipitation/ |
| 39/ | | (0.3 mL) | | | (4° C.) | evaporation |
| B | | | | | | |
| FIG. | 362.3 | ACN | Water | 70 | Fast | precipitation/ |
| 40/ | | (0.4 mL) | (0.1 mL) | | (4° C.) | evaporation |
| A | | | | | | |
| FIG. | 358.9 | IPA | Water | 70 | Slow | Clear/ |
| 41/ | | (0.2) | (0.05) | | (20° C./ | evaporation |
| L | | | | | h) | |
| FIG. | 337.0 | Acetone | Water | 50 | Slow | precipitation/ |
| 42/ | | (0.35) | (0.1) | | (20° C./ | evaporation |
| A | | | | | h) | |
| FIG. | 449.8 | IPA | n-Heptane | 70 | Slow | precipitation/ |
| 43/ | | (0.2) | (0.2) | | (20° C./ | evaporation |
| D | | | | | h) | |
| FIG. | 426.2 | Acetone | c-Hexane | 50 | Fast | precipitation/ |
| 44/ | | (0.35) | (0.6) | | (4° C.) | evaporation |
| H | | | | | | |
| FIG. | 425.4 | ACN | c-Hexane | 70 | Slow | precipitation/ |
| 45/ | | (0.5) | (1.5) | | (20° C./ | evaporation |
| L | | | | | h) | |

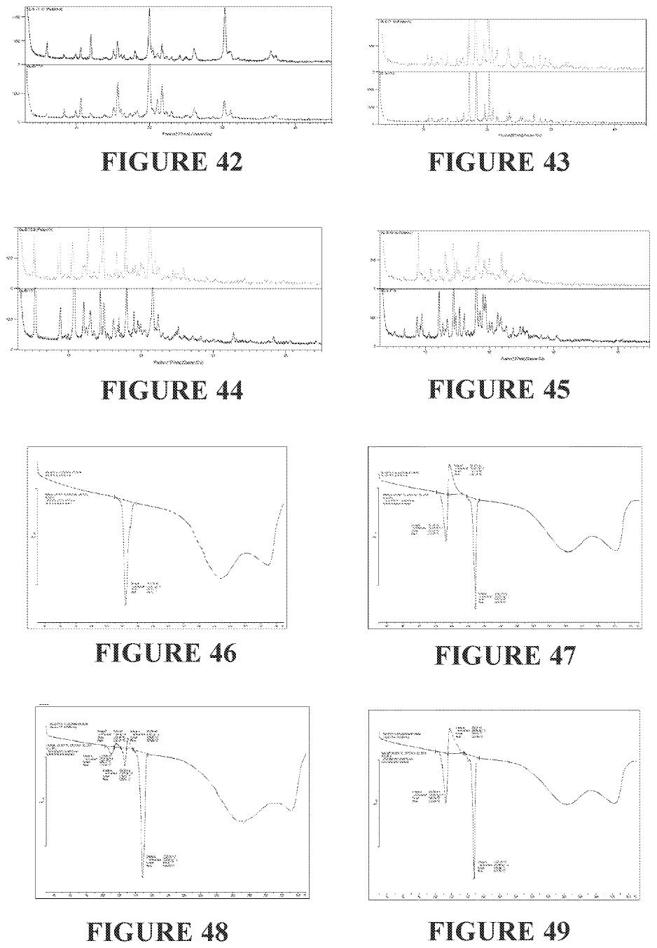

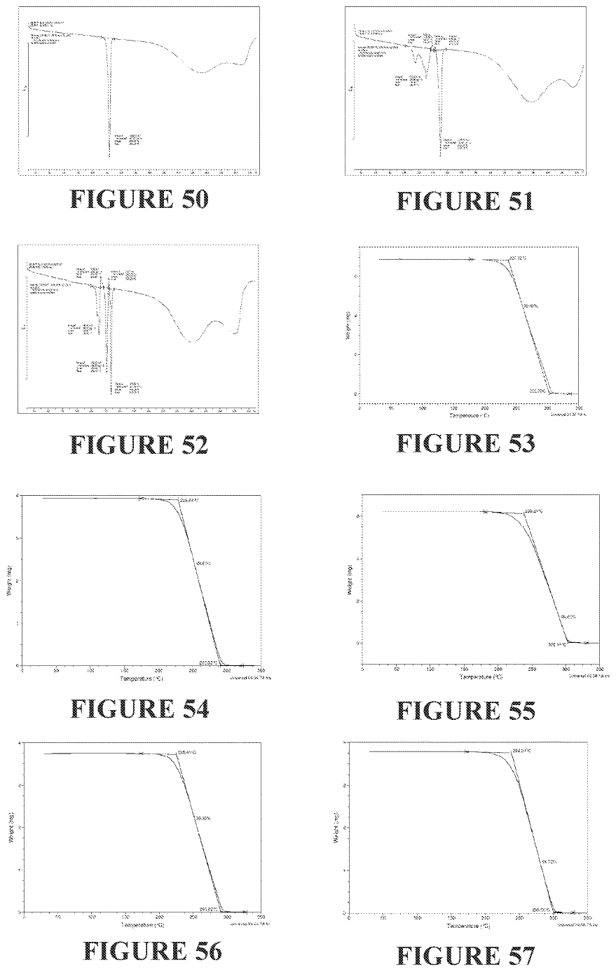

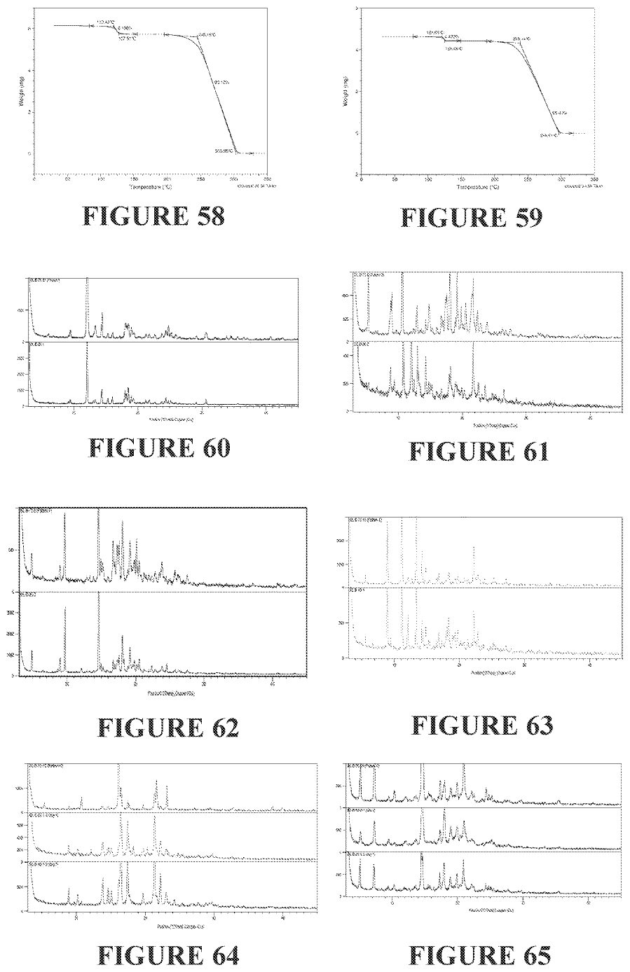

X-ray powder diffraction data of the resultant polymorphs are presented in FIGS. 39-45. In each figure, the top spectrum corresponds to previously generated polymorphs with similar XRD patterns, and the bottom spectrum corresponds to XRD of polymorphs generated from the scale up experiments. DSC and TGA of the polymorphs generated in the scaled-up recrystallizations are presented in FIGS. 46-52 and FIGS. 53-59, respectively.

The DSC data were used to determine the heat of fusion for Forms A, B, D, and F, based on their heats of fusion as measured during conversion from Form C. As outlined in TABLE 10, each of Forms A, B, D, and F are thermodynamically stable, with Form D having the highest stability of these four forms.

TABLE 10

| Pattern | Heat of fusion | Melting Temperature, ° C. |
|---|---|---|
| A | 29.73 | Onset: 106; Peak: 111 |
| B | 53.14 | Onset: 144; Peak: 147 |
| D | 67.51 | Onset: 141; Peak: 143 |
| F | 57.06 | Onset: 148; Peak: 150 |

Since Patterns E, F, G, and I were not obtained, materials from the initial screening experiments were combined for use. The data of combined materials are summarized in TABLE 11. The XRD of these crystallizations (yielding I, G, F, and E, respectively) are shown in FIGS. 60-63, with standard XRD of the targeted spectra provided as the top spectrum in each figure and the obtained spectrum shown at the bottom of each figure.

TABLE 11

| Category | Resultant XRD pattern | DSC | TGA |
|---|---|---|---|
| anhydrate | Pattern A | Pattern A | Pattern A |
| anhydrate | Similar to pattern L | Similar to pattern L | Similar to pattern L |
| anhydrate | Similar to pattern L | Similar to pattern L | Slightly different to pattern L |
| | | | (no wt loss) |
| anhydrate | Pattern A | Pattern A | Pattern A |
| anhydrate | Pattern H | Similar to pattern | Pattern H |
| anhydrate | Pattern B | Similar to pattern B (ratio of | Pattern B |
| | | peaks switched) | |
| stable hydrate | Pattern D | Pattern D | Pattern D |

### Example 7

### Competitive Slurry Experiments

Polymorph stability and interconversions were analyzed with competitive slurry experiments. The competitive slurry (solvent-mediated transformation) experiments were performed on Forms A, B, D, E, F, G, and I generated through scaled-up crystallization (summarized in TABLE 11). The competitive slurry experiments were performed with water, MTBE, and n-heptane, chosen for their low solubility for Formula (I). Briefly, the competitive slurry experiments were performed by suspending one or multiple forms of Formula (I) in 1.0 mL of solvents and incubating at room temperature with stirring. The suspensions were aliquoted at one day and seven days and filtered to isolate the solids. Remaining solid was analyzed by XRD without drying.

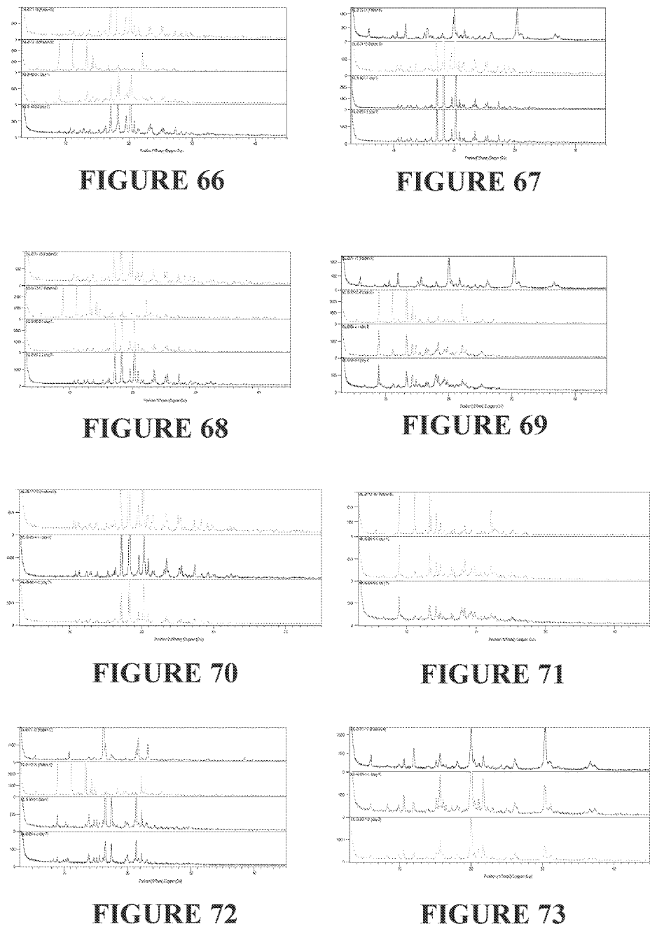

XRD data of the polymorphs in these solvents (water, MTBE, and n-heptane, respectively) are shown in FIGS. 64-66, respectively. The top spectra in FIGS. 64-66 correspond to form C (FIG. 64), K (FIG. 65), and D and E (FIG. 66), respectively, while the spectra second from the bottom and at the bottom of each figure corresponds to Formula (I) in each indicated solvent system after 1 and 7 days at room temperature. In FIG. 66, the second spectrum from the top corresponds to Form E. These XRD data confirmed that Patterns C, K, and D were afforded in these solvents. The data are summarized in TABLE 12.

TABLE 12

| | Actual | | | | |
|---|---|---|---|---|---|
| Starting | wt | | Medium | XRD | XRD |
| Pattern | (mg) | Temp | (Volume mL) | (1 day) | (7 day) |
| A | 6.66 | Room | 1.0 | C | C |
| B | 4.72 | Temperature | (water) | | |
| D | 4.95 | | | | |
| E | 3.11 | | | | |
| F | 4.24 | | | | |
| G | 5.10 | | | | |
| I | 1.45 | | | | |
| A | 7.15 | Room | 1.0 | K | K |
| B | 5.43 | Temperature | (MTBE) | | |
| D | 5.82 | | | | |
| E | 3.42 | | | | |
| F | 3.93 | | | | |
| G | 3.16 | | | | |
| I | 1.25 | | | | |
| A | 6.75 | Room | 1.0 | E | D |
| B | 4.53 | Temperature | (n-heptane) | (minor D) | |
| D | 7.59 | | | | |
| E | 4.17 | | | | |
| F | 8.70 | | | | |
| G | 3.15 | | | | |
| I | 1.70 | | | | |

The competitive slurry experiments indicated that Pattern C (formed in water) was an unstable hydrate. The pattern K from MTBE slurry suggested that it is an MTBE solvate since it was only produced in the singe-solvent crystallization using MTBE as solvent by both slow and fast cooling process and residual MTBE was detected by NMR. Conversion from polymorph mixture to single Pattern D (an anhydrous form of Formula (I)) in n-heptane suggested that Pattern D was the most stable form at RT.

In order to further confirm the rank of stability order of the selected forms and extend the temperature range of stability relationship up to 70° C. covering potential API process conditions, binary form slurries were conducted with starting mixtures of (1) Forms A and D, and (2) Forms D and E. The resultant polymorphs were characterized with XRD after 1 and 7 days, the results of which analyses are shown in FIGURE S 67-68 and summarized in TABLE 13. Pattern D stood out from both binary mixture slurries of A & D and of D & E after seven-day stirring at the elevated temperature. Therefore, it is concluded that Form D was the most thermodynamically stable between room temperature and 70° C.

TABLE 13

| Starting | Formula (I) | | Medium | XRD | XRD |
|---|---|---|---|---|---|
| Pattern | (mg) | Temp | (Volume, mL) | (1 day) | (7 day) |
| A | 20 | 70° C. | 1.5 | D | D |
| D | 26 | | (n-heptane) | | |
| D | 26 | 70° C. | 1.5 | D | D |
| E | 27 | | (n-heptane) | | |

Additional single form slurry experiments were conducted on Forms A, D, and E in either water or heptane at room temperature. The resultant polymorphs were analyzed with XRD following 1 and 7 days, the results of which are provided in FIGS. 69-72. In FIG. 69, the top two spectra correspond to the Formula (I) starting material (Form A) and product form E, respectively, while the bottom two spectra correspond to XRD spectra of Formula (I) following land 7 days in n-heptane, respectively. In FIG. 70, the top spectrum corresponds to the Formula (I) starting material (Form A), the middle spectrum corresponds to Formula (I) following 1 day in n-heptane, and the bottom spectrum corresponds to Formula (I) following 7 days in n-heptane. In FIG. 71, the top spectrum corresponds to the Formula (I) starting material (Form E), the middle spectrum corresponds to Formula (I) following 1 day in n-heptane, and the bottom spectrum corresponds to Formula (I) following 7 days in n-heptane. In FIG. 72, the top spectrum corresponds to the product (Form C) XRD pattern, the second from the top spectrum corresponds to the Formula (I) starting material (Form E), the second spectrum from the bottom corresponds to Formula (I) following 1 day in n-heptane, and the bottom spectrum corresponds to Formula (I) following 7 days in n-heptane. The competitive slurry experiments and resultant polymorphs are summarized in TABLE 14. In agreement with prior competitive slurry experiments, Form D was stable at room temperature in n-heptane. Pattern E was obtained from slurries starting with Forms A and E in n-heptane. As expected, Form C was formed from the Pattern E slurry in water.

TABLE 14

| Starting | Formula (I) | | Medium | XRD | XRD |
|---|---|---|---|---|---|
| Pattern | (mg) | Temp | (Volume, mL) | (1 day) | (7 day) |
| A | 30 | Room | 1.0 | E | E |
| | | Temperature | (n-heptane) | | |
| D | 31 | Room | 1.0 | D | D |
| | | Temperature | (n-heptane) | | |
| E | 31 | Room | 1.0 | E | E |
| | | Temperature | (n-heptane) | | |
| E | 29 | Room | 1.0 | C | C |
| | | Temperature | (water) | (minor E) | |

### Example 8

### Thermal Stress Studies

This example covers thermal stabilities of multiple polymorphs of Formula (I). Seven forms of Formula (I) were incubated at elevated temperature to investigate polymorph stabilities and heat-mediated polymorph transformations. Briefly, 20 mg of solid Form A, B, C, D, E, F, G, and I were weighed into 7 mL glass vials. The vials were covered with tissue and stored in ovens set to 60° C. and ambient pressure. Following 1 and 7 days incubation, solid was collected for XRD analysis.

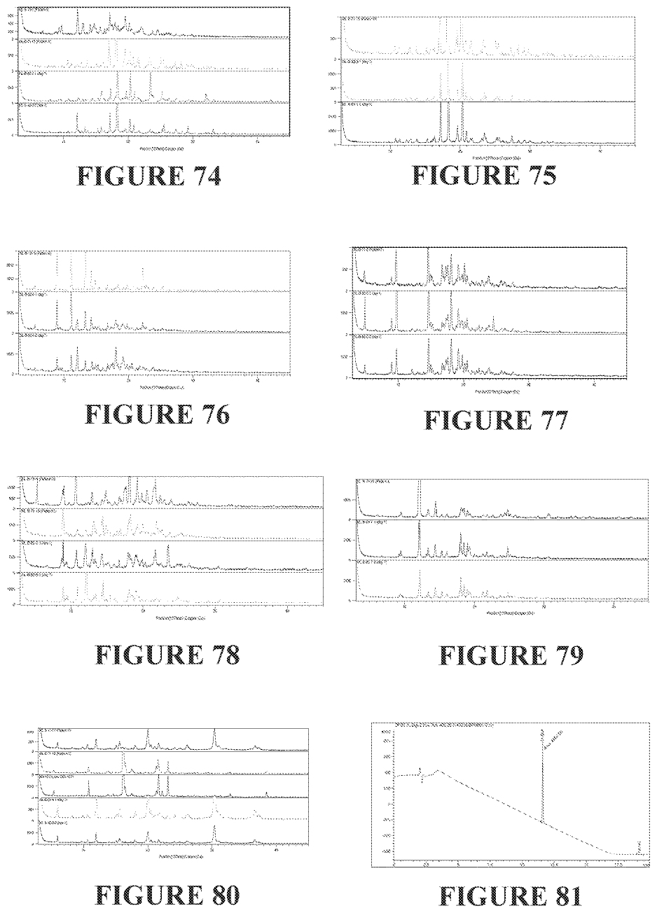

Results of the thermal stress analyses are summarized in TABLE 15. XRD data of the pre- and post-thermal stress polymorphs are presented in FIGS. 73-80, with the top spectrum in each figure corresponding to pre-thermal stress XRD, the middle spectrum in each figure providing XRD following 1 day of thermal stress, and the bottom spectrum in each figure providing XRD after 7 days of thermal stress at the temperature indicated in TABLE 15, and FIGS. 73-80 providing thermal stress progressions for starting Formula (I) Forms A, D, D, E, F, G, I, and A, respectively. As shown in these figures, Forms B and C converted to Forms D and A, respectively, while the other forms remained unchanged.

TABLE 15

| Formula | | | | | | | |
|---|---|---|---|---|---|---|---|
| (I) | Temper- | Initial | | Day 1 | | Day 7 | |
| (mg) | ature | XRD | % AUC | XRD | % AUC | XRD | % AUC |
| 26.66 | 60° C. | A | 100% | A | 100% | A | 100% |
| 21.15 | 60° C. | D | 100% | D | 100% | D | 100% |
| 24.46 | 60° C. | D | 100% | D | 100% | D | 100% |
| 15.30 | 60° C. | F | 100% | F | 100% | E | 100% |
| 24.32 | 60° C. | F | 100% | F | 100% | F | 100% |
| 23.70 | 60° C. | G | 100% | G | 100% | G/L | 100% |
| 10.27 | 60° C. | I | 100% | I | 100% | I | 100% |
| 27.23 | 60° C. | A | 100% | A | 100% | A | 100% |

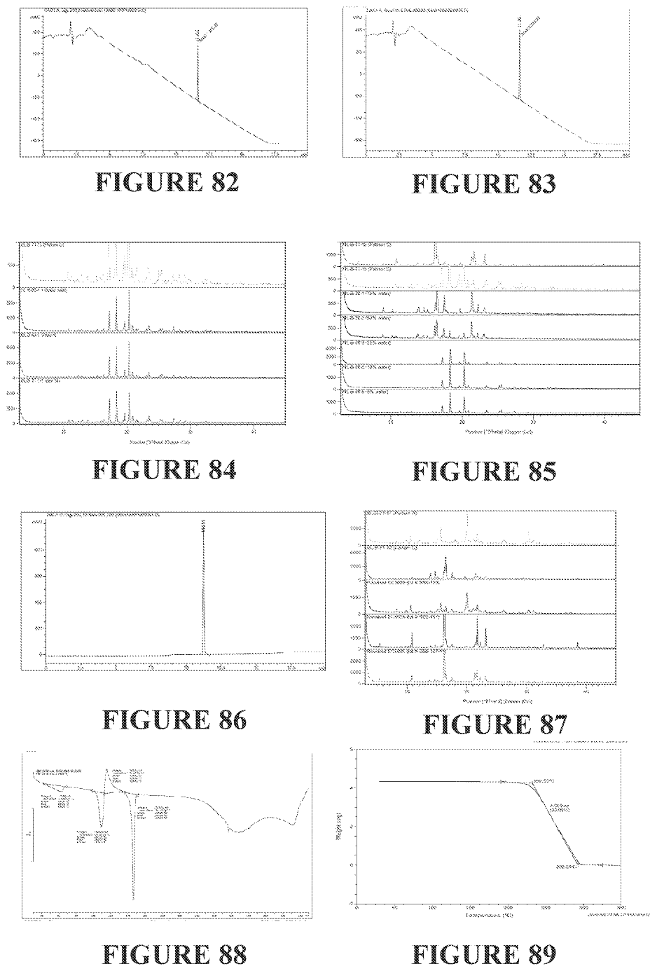

The starting materials, intermediates (1 day of thermal stress), and products (7 days of thermal stress) were analyzed by H PLC. Representative chromatograms from the Form D thermal stress analyses are shown in FIGS. 81-83 (starting material, 1 day, and 7 day samples, respectively). These data suggest that no impurity was observed for all of the samples during seven-day thermal stress at 60° C.

### Example 9

### Hygroscopic Analyses

To further evaluate physical stability, Form D was tested for stability in humid conditions. Form D was placed in an open vial and equilibrated at 75% relative humidity and 40° C. FIG. 84 summarizes XRD analyses used to monitor the polymorph over the 14-day experiment. In this figure, the top panel provides a representative XRD spectrum of Form D, the second from the top panel is an XRD spectrum of the starting material, the third from the top panel is an XRD spectrum of the starting material after 4 days of humidity exposure, and the bottom panel is an XRD spectrum of the starting material after 14 days of humidity exposure. The XRD data show that Form D was unchanged after 4 and 14 days equilibration, and suggest that Form D has extended air and humidity-stabilities.

### Example 10

### PEG-Stability Analyses

As Formula (I) may be formulated in PEG (e.g., as a suspension or solution), the stability of various Formula (I) forms were evaluated in PEG300. Of particular interest was whether Form D would convert to a hydrate (e.g., Form C) in the presence of water. To investigate the critical water concentration for phase interconversion between Pattern C and D, a series experiments were designed (as summarized in TABLE 16). A 1:1 mixture of Forms C and D was prepared and slurried in PEG 300 with different levels of water concentrations. Phase transformations were monitored with XRD, and are summarized in FIG. 85, which (from top to bottom) provides XRD of Form C, Form D, the mixture following incubation in PEG300 containing 75% water, the mixture following incubation in PEG300 containing 50% water, the mixture following incubation in PEG300 containing 25% water, the mixture following incubation in PEG300 containing 10% water, and the mixture following incubation in PEG300 containing 5% water. The data showed a critical water concentration at 50%. Form D is the stable form in PEG300 with less than 50% water. However, in PEG300 solutions with 50% or higher water content, Form C became more stable and conversion to Form C was observed resulting in either a mixture of Form C and Form D or pure Form C.

TABLE 16

| | Starting XRD | XRD Pattern at |
|---|---|---|
| Medium | pattern | Equilibrium |
| H₂O:PEG 300 = 75%:25% | C (50%) + D (50%) | C |
| H₂O:PEG 300 = 50%:50% | C (50%) + D (50%) | C (major) + D (minor) |
| H₂O:PEG 300 = 25%:75% | C (50%) + D (50%) | D |
| H₂O:PEG 300 = 10%:90% | C (50%) + D (50%) | D |
| H₂O:PEG 300 = 5%:95% | C (50%) + D (50%) | D |

### Example 11

### Chemical Stability Analyses

The chemical stability study of Form D of Formula (I) was conducted in various pH media and selected excipients at both RT and 50° C. for up to 1 week. For the pH stability analyses, approximately 2-3 mg Form D was incubated in 1.0 mL of aqueous pH 1.0 (0.1 M HCl), pH 6.8 (water adjusted using 0.1 M NaOH and 0.1 M HCl), and pH 10.0 solutions. No impurities were detectable following 4 or 7 days of incubation at room temperature or 50° C. at any of the pH s tested.

For the excipient stability analyses, approximately 10-20 mg Form D was incubated in PEG400, propylene glycol, D-a-Tocopherol polyethylene glycol 1000 succinate (Vitamin E TPGS), Gelucire® 44/14, hydrogenated castor oil, glyceryl caprylate/caprate(Capmul® MCM), ethoxylated solubilizers (Kolliphor® HS 15, Kolliphor® EL), nonionic surfactant (Labrasol), and diethylene glycol monoethyl ether (Transcutol® HP). Impurities resulting from the incubations were monitored by H PLC chromatography, and are summarized in TABLES 17-18. A representative HPLC chromatogram is shown in FIG. 86. No impurities were observed after either 4 or 7 days of incubation at room temperature or 50° C. These data suggest that Form D of Formula (I) is chemically compatible with a wide range of pH media and pharmaceutically acceptable excipients.

TABLE 17

| | | Starting Form of | Impurity, % | Impurity, % |
|---|---|---|---|---|
| Condition | pH | Formula (I) | (Day 4) | (Day 7) |
| Room | 1.0 | Form D | None Detected | None Detected |
| Temperature | 6.8 | Form D | None Detected | None Detected |
| | 10.0 | Form D | None Detected | None Detected |
| 50° C. | 1.0 | Form D | None Detected | None Detected |
| | 6.8 | Form D | None Detected | None Detected |
| | 10.0 | Form D | None Detected | None Detected |

TABLE 18

| | | Starting Form of | Impurity, % | Impurity, % |
|---|---|---|---|---|
| Condition | Excipient | Formula (I) | (Day 1) | (Day 3) |
| Room | PEG 400 | Form D | None | — |
| Temperature | | | Detected | |
| | Propylene glycol | Form D | None | None |
| | | | Detected | Detected |
| | Vitamin E TPGS | Form D | None | None |
| | | | Detected | Detected |
| | Gelucire 44/14 | Form D | None | None |
| | | | Detected | Detected |
| | Castor Oil, | Form D | None | None |
| | hydrogenated* | | Detected | Detected |
| | Capmul MCM, NF | Form D | None | None |
| | | | Detected | Detected |
| | Capmul MCM, EP | Form D | None | None |
| | | | Detected | Detected |
| | Kolliphor HS-15 | Form D | None | None |
| | | | Detected | Detected |
| | Kolliphor EL | Form D | None | None |
| | | | Detected | Detected |
| | Labrasol | Form D | None | None |
| | | | Detected | Detected |
| | Transcutol | Form D | None | None |
| | | | Detected | Detected |
| 50° C. | PEG 400 | Form D | None | — |
| | | | Detected | |
| | Propylene glycol | Form D | None | None |
| | | | Detected | Detected |
| | Vitamin E TPGS | Form D | None | None |
| | | | Detected | Detected |
| | Gelucire 44/14 | Form D | None | None |
| | | | Detected | Detected |
| | Castor Oil, | Form D | None | None |
| | hydrogenated | | Detected | Detected |
| | Capmul MCM, NF | Form D | None | None |
| | | | Detected | Detected |
| | Capmul MCM, EP | Form D | None | None |
| | | | Detected | Detected |
| | Kolliphor HS-15 | Form D | None | None |
| | | | Detected | Detected |
| | Kolliphor EL | Form D | None | None |
| | | | Detected | Detected |
| | Labrasol | Form D | None | None |
| | | | Detected | Detected |
| | Transcutol | Form D | None | None |
| | | | Detected | Detected |

### Example 12

### Characterizations of Formula (I) Starting Materials

Three batches of Formula (I) were analyzed for purity prior to crystallization and polymorph analyses presented in previous examples. Each batch included either Form A or Form C. Baseline characterizations of the batches included DSC, TGA, microscopic analysis, and vapor sorption, the results of which are summarized in TABLE 19. FIG. 87 provides XRD of representative Form A and Form C lots (top and second from top, respectively) above XRD of the three batches of Formula (I). Lot 1 primarily contained Form A, while lots 2 & 3 primarily contained Form C.

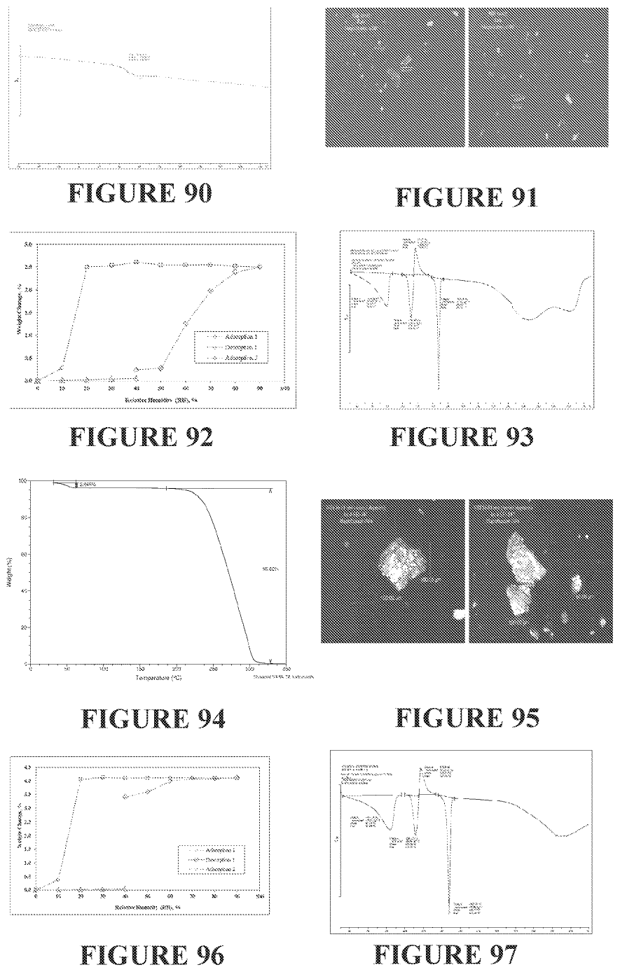

FIGS. 88-92 provide physical analyses of lot 1. Briefly, FIG. 88, a DSC thermogram of lot 1, and includes peaks corresponding to Forms A and B. FIG. 89 provides a TGA thermogram of lot 1, with 0% weight change below 100° C. and 99% weight change at 233° C. indicating high purity and low water content. FIG. 90 provides a modulated DSC thermogram of lot 1, and identifies a glass transition temperature midpoint of around 48.5° C. FIG. 91 provides two polarized-light microscopic images of lot 1, and shows that Formula (I) is primarily microcrystalline with about 1-50 μm crystals. FIG. 92 provides dynamic vapor sorption plots of lot 1, indicating 2.49 weight percent water vapor adsorption at 90% relative humidity, 1.25 weight percent water vapor adsorption at 60% relative humidity, and close to complete water vapor desorption at 0% relative humidity.

FIGS. 93-96 provide physical analyses of lot B. FIG. 93 provides a DSC thermogram of lot 2, exhibiting a low temperature peak corresponding to the desorption of bound water (from Form C), as well as higher temperature peaks corresponding to melting of forms A and B. FIG. 94 provides a TGA thermogram of lot 2, showing a 2.9% weight change below 100° C. corresponding to water desorption and a 96% weight change at 233° C. corresponding to melting of Formula (I), confirming the presence of Form C and indicating high purity of Formula (I). FIG. 95 provides polarized-light microscope images of lot 2 at 200× magnification, with 50 and 100 μm scales shown. The images show that lot 2 contained a mixture of microcrystals spanning individual microns to multiple hundreds of microns in size. FIG. 96 provides dynamic vapor sorption plots of lot 2, indicating 3.99 weight percent water vapor adsorption at 60% relative humidity, 4.12 weight percent water vapor adsorption at 90% relative humidity, and close to complete water vapor desorption at 0% relative humidity.

FIG. 97 provides a DSC thermogram of lot 3, and includes a large endothermic peak at 84° C. corresponding to water desorption, as well as melting and crystallization peaks of Forms A and B, indicating that lot 3 includes of mixture of Forms A, B, and C.

TABLE 19

| Lot/ | | | Impurity, % | Impurity, % |
|---|---|---|---|---|
| XRD | Excipient | Lot # | (Day 1) | (Day 3) |
| Lot 1/ | Endotherm; | 0% wt change | Irregular plate; | 60% RH 1.25 wt % |
| Pattern | 63° C. | below 100° C. | Size range; 5-80 μm; | 90% RH 2.49 wt % |
| A | (likely dehydration of bound | 99% wt change | Birefringent | |
| | water), | at 233° C. | | |
| | Exotherm; | | | |
| | 115° C. (crystallization of | | | |
| | Form B) | | | |
| Lot 2/ | Endotherm; | 2.9% wt change | Irregular plate; | 60% RH 3.99 wt % |
| Pattern | 79° C. (likely dehydration of | below 100° C. | Size range; 5-200 μm; | 90% RH 4.12 wt % |
| C | bound water), | 96% wt change | Birefringent | |
| | 111° C. (melting of Form A), | at 233° C. | | |
| | Exotherm; | | | |
| | 117° C. (crystallization of | | | |
| | Form B) | | | |
| Lot 3/ | Endotherm; | NA | NA | NA |
| Pattern | 84° C. | | | |
| C | (likely dehydration of bound | | | |
| | water), | | | |
| | 111° C. (melting of Form A), | | | |
| | Exotherm; | | | |
| | 117° C. (crystallization of | | | |
| | Form B) | | | |

### Example 13

### Characterizations of Formula (I) Starting Materials

This example covers the thermal transformation of Form B to Form D as a scalable route for Form D manufacture. In EXAMPLE 4, Form D was produced by evaporation, which, for some crystallizations, is not as readily scalable as other alternative crystallization methods. Results of the thermal stress study outlined in EXAMPLE 8 suggested that Form D can be obtained by solid-to-solid transformation from pattern B through incubation at elevated temperatures.

Several scaled up crystallization conditions are summarized in TABLE 20, each of which utilized Form C as a starting material. In one crystallization, Form B was obtained in a fast cooling crystallization in ACN. Therefore, preparation of Form B was further attempted in fast cooling ACN. However, as shown in TABLE 20 fast cooling crystallization in ACN gave Form D directly.

TABLE 20

| | For- | Primary | Anti- | | | | |
|---|---|---|---|---|---|---|---|
| | mula | Solvent | solvent | | | Pre- | |
| | (I) | (Vol, | (Vol, | Temp | | cipitation/ | |
| Prep. | (mg) | mL) | mL) | (° C.) | Cooling | Isolation | XRD |
| 1 | 313 | ACN (0.4) | — | 70 | Fast | ppt/filter | D |
| | | | | | (4° C.) | | |
| 2 | 318 | ACN (0.4) | c-Hexane | 70 | Slow | ppt/filter | H |
| | | | (5) | | (20° C./ | | |
| | | | | | h) | | |
| 3 | 299 | Dioxane | c-Hexane | 70 | Slow | ppt/filter | G |
| | | | (8) | | (20° C./ | | |
| | | | | | h) | | |
| 4 | 313 | ACN (0.4) | — | 70 | Fast | ppt/filter | D |
| | | | | | (4° C.) | | |
| 5 | 270 | ACN (0.4) | — | 70 | Fast | ppt/filter | D |
| | | | | | (4° C.) | | |
| 6 | 290 | ACN (0.3) | — | 70 | Slow | ppt/filter | D |
| | | | | | (20° C./ | | |
| | | | | | h) | | |

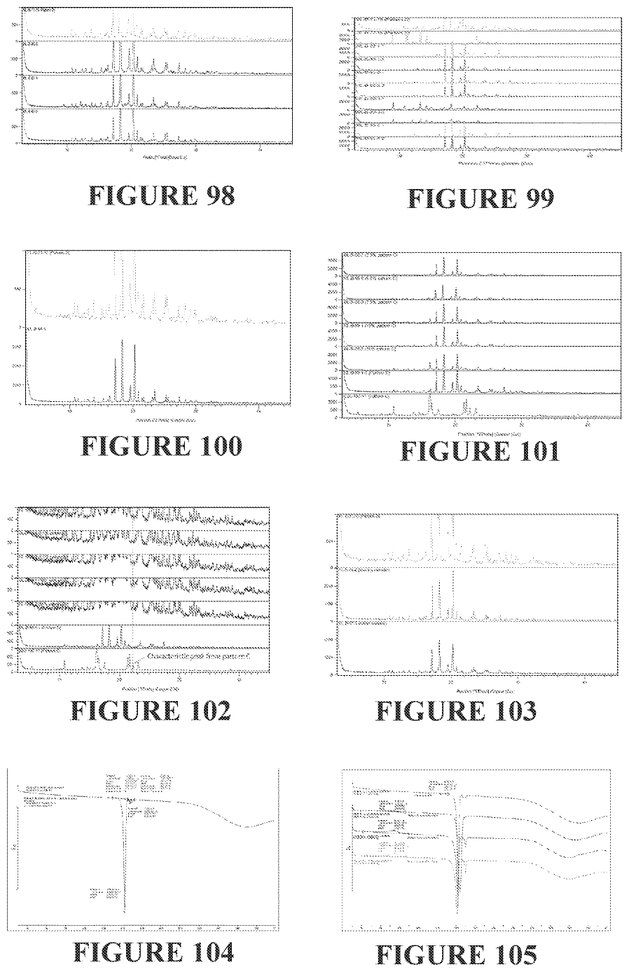

Because fast cooling can be difficult to achieve on scale, crystallization in ACN was optimized using slow cooling. Results of multiple ACN crystallizations, summarized in TABLE 24 and the XRD spectra of FIG. 98 (from top to bottom showing XRD spectra of Form D, TABLE 20 preparation 4, TABLE 20 preparation 5, and TABLE 20 preparation 6), showed that Form D can be produced from ACN solvent crystallization by both fast and slow cooling. However, from process development point of view, the ACN process had two drawbacks. One is that solvent to API volume ratio is often low, which can lead to a very concentrated process and poor material transfer and recovery. The other is that ACN is an ICH Class II solvent with low residual solvent limit. Accordingly, use of ICH Class III solvents in place of ACN may be preferred in some pharmaceutical preparations.

Several additional crystallization conditions using seeding and binary solvent systems were tested. As summarized in TABLE 21, these crystallizations utilized ethanol, isopropyl alcohol, methylethylketone, and ethyl acetate as primary solvents, n-heptane as an antisolvent, initial temperatures of 70° C., and cooling rates of 20° C./hour. Two replicates were performed with each condition. XRD of the resultant polymorphs are shown in FIG. 99, which from top-to-bottom provides XRD of Form D, Form E, the two replicates of the ethanol recrystallizations, the two replicates of the isopropyl alcohol recrystallizations, the two replicates of the two methylethylketone recrystallizations, and the two replicates of the ethyl acetate recrystallizations, respectively. Form D solids were successfully produced from systems with primary solvent using EtOH, IPA, and EtOAc, and n-heptane as antisolvent. Form E was produced using methylethylketone as primary solvent and n-heptane as secondary solvent.

TABLE 21

| Formula | | | | | | XRD Pattern |
|---|---|---|---|---|---|---|
| (I) | | | | | | Following |
| Starting | Primary | Anti- | Temp | Cooling | XRD | Extra n-heptane |
| Form | Solvent | solvent | (° C.) | Rate | Pattern | Addition |
| C | EtOH | n-heptane | 70 | 20° C./h | D | D |
| | IPA | n-heptane | 70 | 20° C./h | D | D |
| | MEK | n-heptane | 70 | 20° C./h | E | E |
| | EtOAc | n-heptane | 70 | 20° C./h | D | D |

The solubility of Form C of Formula (I) in the binary solvent systems of TABLE 21 are summarized in TABLE 22. When ethanol was used as a primary solvent, Formula (I) solubility decreased with increasing n-heptane solvent content. For isopropyl alcohol, methylethylketone, and ethyl acetate, Formula (I) solubility increased with increasing n-heptane solvent content.

TABLE 22

| | 1^{st} test point | | 2^{nd} test point | | 3^{rd} test point | |
|---|---|---|---|---|---|---|
| | Ratio | | Ratio | | Ratio | |
| | of n- | | of n- | Solu- | of n- | Solu- |
| Primary | heptane | | heptane | bility | heptane | bility |
| solvent in | to | Solubility | to | (mg/ | to | (mg/ |
| Experiment | primary | (mg/mL) | primary | mL) | primary | mL) |
| EtOH | 7.7 | 38 | 13.2 | 10 | n/a | n/a |
| IPA | 13.3 | 30 | 13.9 | 10 | 21.3 | 5 |
| MEK | 2.0 | 11 | 3.0 | 5 | 3.4 | 4 |
| EtoAC | 1.4 | 7 | 2.0 | 4 | 2.2 | 3 |

### Example 14

### Gram-Scale Polymorph Stallization

In order to have sufficient amount of Form D for solubility testing and micronization, seeding crystallizations were performed at gram scale. Specifically, 27 g of Form C was dissolved in EtOH (15 mL) at 70° C. in a 500 mL flask equipped with stir bar. n-Heptane was added in 20 mL aliquots until the solution turned slightly turbid (220 mL n-heptane added in total). The mixture was stirred at 70° C. to form a clear solution. The batch was cooled to room temperature at 20° C. per hour down to a final temperature of 40° C. Simultaneously, 2-3 milligrams of Form D seed crystal were added in five minute intervals until precipitation was observed. The solid was isolated by filtration and dried at RT in vacuum oven, yielding 18.17 g of solids (67% yield). FIG. 100 provides XRD of Form D (top) and the crystals obtained from the scaled up seeding crystallization (bottom). As shown in this figure, XRD of the resultant crystals was consistent with Form D.

### Example 15

### X-Ray Powder Diffraction Detection Limits

The detection limit of Form C in Form D with XRD was evaluated to determine the minimum purities of Form D generated in the scale-up crystallizations. To perform these analyses, a series of blends with different levels of Pattern C from 2.5% to 15% were analyzed by XRD. The characteristic peak of Pattern C at 22.2 degree of 2-theta was used to quantify Form C in each mixture. XRD of the Form C, Form D mixtures are provided in FIGS. 101 and 102. In each figure, from top to bottom, the XRD spectra correspond to 2.5% Form C and 97.5% Form D (top), 5% Form C and 95% Form D (second from top), 7.5% Form C and 92.5% Form D (third from top), 10% Form C and 90% Form D (fourth from top), 15% Form C and 85% Form D (fifth from top), pure Form D (second from bottom), and pure Form C (bottom). FIG. 102 provides a zoomed-in version of the XRD spectra provided in FIG. 101. As the 22.2 degree 2-theta peak (corresponding to Form C) is discernible from mixtures with 7.5% and higher proportions of Form C, additional physical characterization techniques (e.g., detection of low temperature water desorption in differential scanning calorimetry) may be needed to quantify low levels of Form C in Form D preparations.

### Example 16

### Solubility Enhancement-Screening

This example covers the solubility of Formula (I) polymorphs in various solvent systems. As Formula (I) often has limited aqueous solubility, strategies for increasing its solubility could be used to enhance its dissolution and bioavailability upon administration. On this basis, several experiments were conducted, including particle size reduction and amorphous formulation, to assess various solubility enhancement approaches for optimized drug delivery.

### Equilibrium-Solubility Screening

Formula (I) equilibrium solubility was tested in a range of solvent and excipient systems. For these analyses, Form D or Form C was mixed with 0.5 or 1.0 mL of solvents and equilibrated for approximately 24 hours. The mixture was filtered through 0.45 μm centrifuge filters. Solid Formula (I) collected during filtration analyzed by XRD to determine the remaining solid polymorphic forms, while the filtrate was collected for analysis by H PLC to determine solubility in each solvent.

The equilibrium solubility data are summarized in TABLE 23. As expected, the compound was not easily ionized at physiologically relevant pH. Nonetheless, a number of excipients with greater than 20 mg/mL Formula (I) solubilities were identified, including PEG300, PEG400, propylene glycol, vitamin E TPGS, Gelucire 44/14, castor oil, hydrogenated, Capmul MCM, NF, Capmul MCM, EP, Kolliphor HS-15, Kolliphor EL, Labrasol, and Transcutol HP. In addition, these excipients included surfactants, co-solvents, and lipids which are amenable for use in self-emulsifying drug delivery systems (SEDDS). The XRD pattern of the starting material was maintained for each sample after equilibrium.

TABLE 23

| Formula (I) | | Equilibrium | XRD Pattern At |
|---|---|---|---|
| Starting Form | Medium | Solubility (mg/mL) | Equilibrium |
| Form D | DI water | <0.1 | Form C (minor |
| | | | Form D) |
| Form D | pH 1.0 HCl | <0.1 | Forms C & D |
| Form D | pH 7.4 | <0.1 | Form D |
| Form D | pH 10.0 NaOH | <0.1 | Forms C & D |
| Form D | PEG 300, 50% | 0.4 | Form D |
| Form D | PEG 300, 100% | 3.6 | Form D |
| Form D | PEG 400, 50% | 0.4 | Form D |
| Form D | PEG 400, 100% | 20.1 | Form D |
| Form D | PEG 300, 50% | 0.5 | Form D |
| Form D | PEG 300, 100% | 16.9 | Form D |
| Form D | PEG 400, 50% | 0.9 | Form D |
| Form D | PEG 400, 100% | 17.0 | Form D |
| Form C | PEG 300, 50% | 0.8 | Form C |
| Form C | PEG 300, 100% | 16.7 | Form D |
| Form C | PEG 400, 50% | 0.4 | Form C |
| Form C | PEG 400, 100% | 17.9 | Form D |
| Form D | FaSSIF | <0.1 | Form D |
| Form D | FaSSIF | 0.2 | Form D |
| Form D | Tween 80, 100x | <0.1 | Form C |
| Form D | Tween 80, 10x | <0.1 | Form C |
| Form D | Tween 80, 5x | <0.1 | Form C |
| Form D | Tween 80, 1x | <0.1 | Form C |
| Form D | SLS, 100x CMC | 2.5 | Form C |
| Form D | SLS, 10x CMC | 1.5 | Form C |
| Form D | SLS, 5x CMC | 1.2 | Form C |
| Form D | SLS, 1x CMC | <0.1 | Form C |
| Form D | Propylene | 24.1 | Form D |
| Form D | Sulfobutylether | 7.9 | Form D |
| Form D | Vitamin E | 40.6** | Form D |
| Form D | Gelucire 44/14 | 37.7** | Form D |
| Form D | Captex 355, | 2.0 | Form D |
| Form D | Castor Oil | 5.5 | Form D |
| Form D | Castor Oil, | 42.6** | Form D |
| Form D | Soybean Oil | <0.1 | Form D |
| Form D | Corn Oil | <0.1 | Form D |
| Form D | Canola Oil | <0.1 | Form D |
| Form D | Miglyol 810 | 2.0 | Form D |
| Form D | Miglyol 812 | 1.9 | Form D |
| Form D | Capmul MCM, | 24.5 | Form D |
| Form D | Campul MCM | 56.2 | Form D |
| Form D | Kolliphor | 1.4 | Form D |
| Form D | Kolliphor HS- | 37.3** | Form D |
| Form D | Kolliphor EL | 20.4 | Form D |
| Form D | Oleic Acid | 9.0 | Form D |
| Form D | Plurol Oleique | 8.7 | Form D |
| Form D | Peceol | 12.9 | Form D |
| Form D | Labrasol | 21.8 | Form D |
| Form D | Labrafil | 9.6 | Form D |
| Form D | Triacetin | 6.4 | Form D |
| Form D | Transcutol HP | 38.1 | Form D |
| Form D | Glycerol | <0.1 | Form D |
| Form D | Intralipid | 0.1 | Form D |

### Supersaturation Monitoring in PEG 300

The supersaturated solubility of both Formula (I) Form D and Pattern C were prepared at 1100 mg/mL and 200 mg/mL in PEG 300, a select solvent in which Formula (I) exhibited high solubility. The experimental details and results of these analyses are summarized in TABLE 24. 100 mg/mL Formula (I) was stable in PEG300 did not exhibit visual precipitation at room temperature until 9 days of incubation, indicating long-term stability in this condition. When prepared at 200 mg/mL in PEG300, Formula (I) readily precipitated overnight, suggesting that Formula (I) is not stable at this higher concentration in PEG 300.

TABLE 24

| Formula (I) | | |
|---|---|---|
| Form, Mass, | Medium | |
| Nominal Conc. | (Volume) | Step → Observation |
| Pattern D, | PEG 300 | 1. RT stirring for 1 hr → suspension |
| 50.2 mg, | (0.5 mL) | 2. 40 C. heating for 0.5 hr → clear solution |
| 100 mg/mL | | 3. RT storage for ~8 days → clear solution |
| | | 4. RT storage for ~9 days → precipitation |
| Pattern D, | PEG 300 | 1. RT stirring for 1 hr →suspension |
| 100.1 mg, | (0.5 mL) | 2. 40 C. heating for 48 hr →suspension |
| 200 mg/mL | | 3. 55 C. heating for <16 hr →clear solution |
| | | 4. RT storage for <3.5 days →precipitation |
| | | 5. 55 C. reheating for overnight →clear solution |
| | | 6. RT storage for overnight →precipitation |
| Pattern C, | PEG 300 | 1. RT stirring for 1 hr →suspension |
| 50.2 mg, | (0.5 mL) | 2. 40° C. heating for 0.5 hr →clear solution |
| 100 mg/mL | | 3. RT storage for ~8 days →clear solution |
| | | 4. RT storage for ~9 days →precipitation |
| Pattern C, | PEG 300 | 1. RT stirring for 1 hr →suspension |
| 50.2 mg, | (0.5 mL) | 2. 40° C. heating for 48 hr →suspension |
| 200 mg/mL | | 3. 55 C. heating for <16 hr →clear solution |
| | | 4. RT storage for <3.5 days →precipitation |
| | | 5. 55 C. reheating for overnight →clear solution |
| | | 6. RT storage for overnight →precipitation |

### Particle Size Analysis

To probe the impact of particle size reduction of Formula (I) on apparent solubility/dissolution for sake of facilitating formulation development, the solubility of Form D of Formula (I) was tested following grinding to smaller particle size. Before milling, Form D was screened through a 30-micron mesh sieve. Then, Form D micronized using a 2-inch jet mill 70 psig feed pressure, 40 psig grinding pressure, and a 4.3 g/min powder feed rate. The micronization process provided a 69% yield and 95.6% crystallinity relative to the unmilled material. The bulk and tapped densities of Form D (including unmilled and milled) was measured before and after tapping by tap density tester, and showed a reduction of density upon micronization. The unmilled form exhibited a bulk density of 0.54 g/mL and a tapped density of 0.61 g/mL, while the micronixed form exhibited a bulk density of 0.20 g/mL and a tapped density of 0.22 g/mL.

XRD and DSC was performed on the micronized Formula (I) to confirm that compound was unchanged during micronization. FIG. 103 provides the results of the XRD analyses, with the top spectrum corresponding to XRD of pure Form D, the middle spectrum corresponding to the starting material, and the bottom spectrum providing an XRD of the micronized product. FIG. 104 provides a DSC thermogram of the micronized material. No changes were detected from either XRD or DSC following micronization.

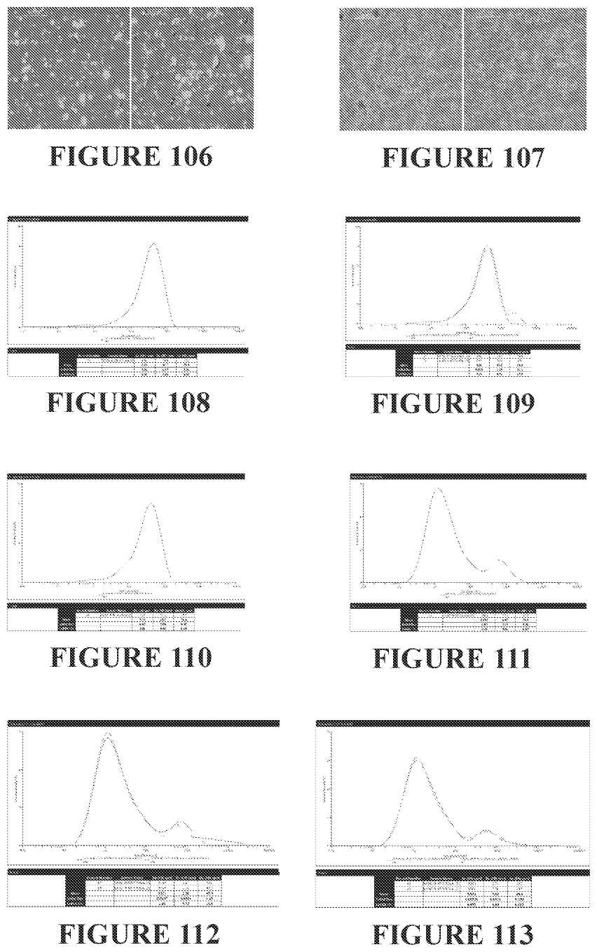

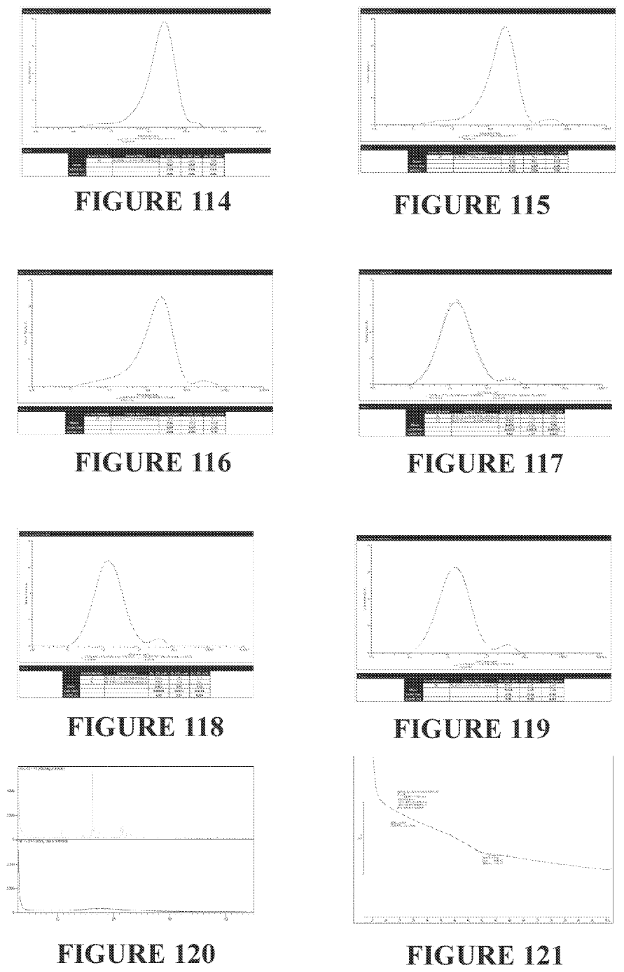

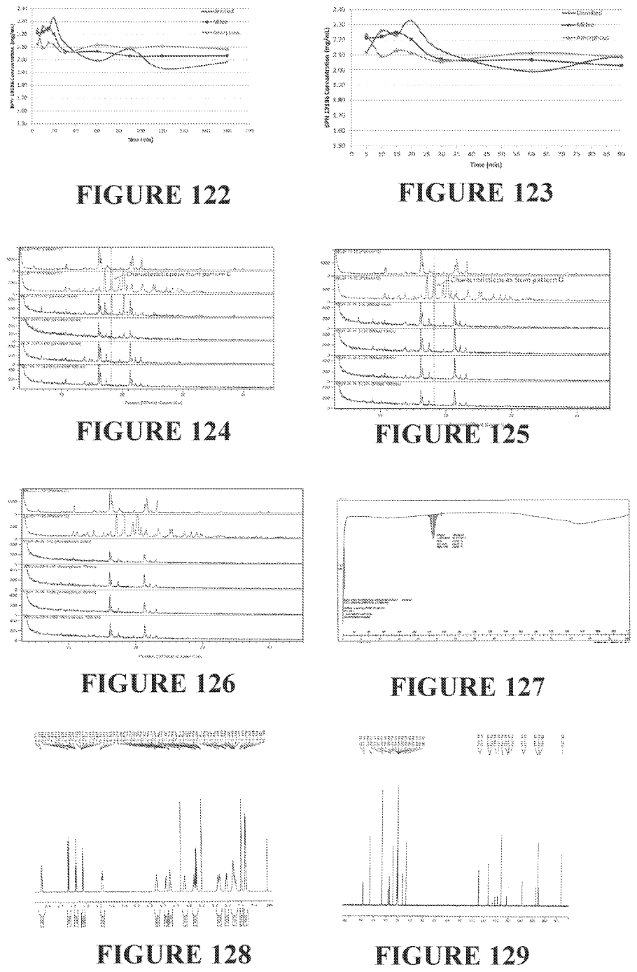

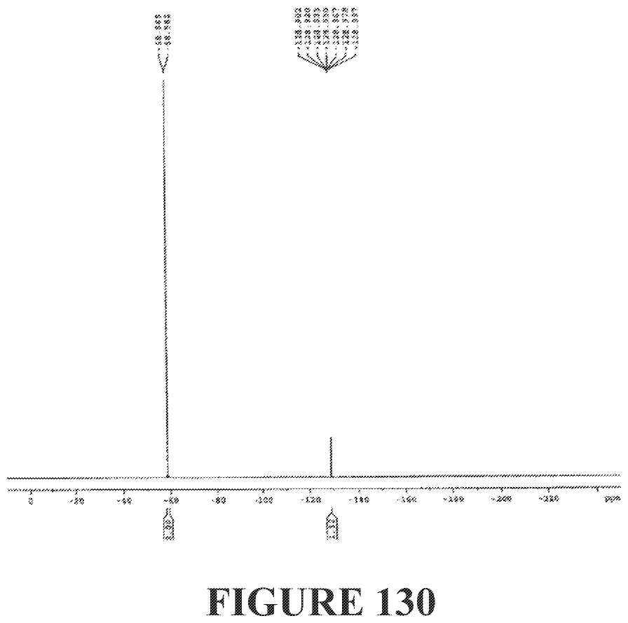

However, small amounts of two new endotherms (at 147° C. and 150° C.) were detected by DSC in the micronized material. The appearance of the two endotherms was likely due to melting of crystalline phases during the DSC scan. To verify the hypothesis of amorphous content conversion (into two endotherms) on DSC, Form D material was ground using mortar and pestle in various degree to generate different levels of amorphous content and analyzed by DSC. FIG. 105 provides the results of these analyses, with the top thermogram corresponding to micronized Form D, the second from the top thermogram corresponding to Form D ground for 5 minutes (in a mortar and pestle), the second thermogram from the bottom corresponding to Form D ground for 10 minutes, and the bottom thermogram corresponding to Form D ground for 15 minutes. DSC endotherms of the ground Form D exhibited similar endotherms at 150° C. The intensity of this endotherm correlated with grinding time, supporting its assignment as melted crystalline phase.

Particle size was first analyzed with optical microscopy. Representative optical microscope images with 200× magnification are shown in FIG. 106 for unmilled Form D and in FIG. 107 for micronized Form D. As can be seen from these images, micronization effectively diminished particle size within the samples.

Particle size was also analyzed with a dry-method using a Malvern 300 particle sizer equipped with a standard energy venturi and utilizing 2.5 bar, 3.0 bar, and 3.5 bar air pressure. The results of these analyses are provided in FIGS. 108-113. FIGS. 108-110 provide results obtained at 2.5, 3.0, and 3.5 bar, respectively, for unmilled Form D, while FIGS. 111-113 provide results obtained at 2.5, 3.0, and 3.5 bar, respectively, for micronized Form D. 10^{th}, 50^{th}, and 90^{th} percentile particle sizes (D₁₀, D₅₀, and D₉₀, respectively) determined at each pressure are summarized for unmilled and micronized Form D in TABLE 25. Notably, the sizing data obtained with the particle sizer was not consistent with the particle sizes determined with optical microscopy. One possible explanation is that the insufficient energy was provided to break up particle agglomerations. Significant variation of D₉₀ for micronized Form D from three air pressure titrations as well as the bimodal profile of the particle size results supports this model, and suggests that Form D particles form relatively strong clusters.

TABLE 25

| Formula | Material | Air | D₁₀ | D₅₀ | D₉₀ | PSD |
|---|---|---|---|---|---|---|
| (I) | Category | Pressure | (μm) | (μm) | (μm) | Modal |
| Form D | Unmilled | 2.5 bar | 8.66 | 34.5 | 79.3 | Unimodal |
| | | 3.0 bar | 8.26 | 35.0 | 91.2 | Unimodal |
| | | 3.5 bar | 7.37 | 33.1 | 78.6 | Unimodal |
| Form D | Milled | 2.5 bar | 0.56 | 1.85 | 45.9 | Bimodal |
| | | 3.0 bar | 0.55 | 1.80 | 47.4 | Bimodal |
| | | 3.5 bar | 0.54 | 1.69 | 29.4 | Bimodal |

Based on these findings, particle size analysis was repeated at 2.5, 3.0, and 3.5 bar using a higher energy venturi to ensure the break-up of the potential agglomerates. The detailed particle sizing data using high-energy venturi are summarized in TABLE 26 and in FIGS. 114-119, with FIGS. 114-116 corresponding to 2.5, 3.0, and 3.5 bar analyses of unmilled Form D and FIGS. 117-119 corresponding to 2.5, 3.0, and 3.5 bar analyses of micronized Form D, respectively. While the data remained bimodal, indicating incomplete break-up of particle agglomerates, the D₉₀ of micronized material measured below 6 μm, in closer agreement with the optical analyses.

TABLE 26

| Formula | Material | Air | D₁₀ | D₅₀ | D₉₀ | PSD |
|---|---|---|---|---|---|---|
| (I) | Category | Pressure | (μm) | (μm) | (μm) | Modal |
| Form D | Unmilled | 2.5 bar | 5.24 | 22.0 | 55.8 | Bimodal |
| | | 3.0 bar | 4.47 | 20.1 | 54.8 | Bimodal |
| | | 3.5 bar | 2.84 | 17.2 | 51.1 | Bimodal |
| Form D | Milled | 2.5 bar | 0.46 | 1.52 | 5.80 | Bimodal |
| | | 3.0 bar | 0.46 | 1.47 | 5.42 | Bimodal |
| | | 3.5 bar | 0.44 | 1.51 | 5.44 | Bimodal |

### Example 17

### Analysis of Amorphous Phase Transitions

This example covers the generation of amorphous forms of Formula (I) under various temperatures and conditions. A s previously presented, the glass transition temperature (T g) of in situ generated amorphous Formula (I) in DSC is 48° C. (FIG. 90 and TABLE 19), suggesting that thermal generation of amorphous Formula (I) is feasible.

As an alternate approach to amorphous Formula (I) production, the bulk amorphous material was reformed through spray drying. 2% Form C (solid concentration) in ethanol was spray dried with a 115-120° C. inlet temperature, a 39-35° C. out temperature, a 90-95% aspirator rate, and a 50-54% pump rate. XRD of the resultant material is provided FIG. 120, with the top spectrum corresponding to primarily Form C starting material, and the bottom spectrum providing spray-dried material, with the lack of peaks in the bottom spectrum indicating that the spray dried material was in amorphous state. DSC of the spray dried material (FIG. 121) indicated a glass transition temperature (Tg) of around 43° C., which was comparable to the Tg of the amorphous material generated in situ in DSC.

### Example 18

### Kinetic Solubility Analyses

The kinetic solubility of Formula (I) provided as unmilled Form D, micronized Form D, and in amorphous form was measured in sodium dodecyl sulfate (SLS) at 60 mM (7× critical micelle concentration) at nominal concentrations of approximately 4 mg/mL (non-sink conditions; see TABLE 23 for Formula (I) SLS solubilities). The mixture was stirred at room temperature with astir bar. The samples (n=3) were collected at each predetermined time points (5, 10, 15, 20, 30, 60, 90, 120, and 180 minutes) and centrifuged. The filtrate was analyzed by H PLC to determine solubility, and the remaining solid was analyzed by XRD as appropriate. Three replicates were performed for each condition.

FIGS. 122-123 summarize SLS kinetic solubility data over 180 and 90 minutes, with each trace corresponding to the average of three replicates. Milled, unmilled, and amorphous Formula (I) exhibited similar kinetic solubilities in SLS. The advantages of achieving higher degree of supersaturation from milled material (by providing larger surface area) and amorphous material (by providing higher solubility) were offset by phase transformation from Form D to Pattern C (i.e. hydrate form).

Conversion of Form D and amorphous Formula (I) during the kinetic solubility analyses were monitored with XRD. FIGS. 124-126 provide XRD spectra of unmilled Form D, milled Form D, and amorphous Formula (I), respectively, over 180 minute solubilizations. In each figure, the top spectrum corresponds to pure Form C, the second figure from the top of the figure corresponds to pure Form D, the third spectrum from the top of the figure corresponds to solid collected following 5 minutes of solubilization, the fourth spectrum from the top corresponds to solid collected following 60 minutes of solubilization, the fifth spectrum from the top corresponds to solid collected following 90 minutes of solubilization, and the bottom spectrum corresponds to solid collected following 180 minutes of solubilization. As indicated by these XRD data, the milled and amorphous materials were converted into Pattern C within 5 min after exposure to the dissolution medium. Possibility due to relatively larger particle size of the unmilled material, the unmilled material exhibited a slower rate of conversion to Form C, fully transitioning to the hydrate form following 60 minutes of solubilization. This might explain the slightly longer maintenance of supersaturation from unmilled material. As a mitigation formulation strategy, the amorphous material could be stabilized in dissolution medium by solid dispersion if needed in future formulation work. The advantage of milled and amorphous approaches to reach high degree of supersaturation was offset by phase transition to hydrate form in kinetic solubility study. In future work, if amorphous form is desired for solubility enhancement, amorphous state could be maintained via amorphous solid dispersion by selecting optimal polymer.

Although the invention has been described with reference to the presently preferred embodiment, it should be understood that various modifications can be made without departing from the spirit of the invention. Accordingly, the invention is limited only by the following claims.

1-6. (canceled)

7. A composition comprising a crystalline form of Formula (I): 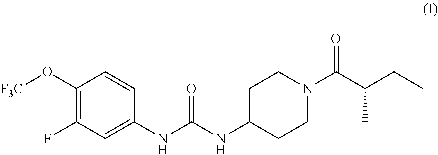 [SMILES: CC[C@H](C)C(=O)N1CCC(NC(=O)Nc2ccc(OC(F)(F)F)c(F)c2)CC1]
or a pharmaceutically acceptable salt thereof,
wherein the crystalline form of Formula (I) comprises Form D as characterized by an X-ray powder diffraction substantially as set forth in Panel D of FIG. 1.

8. The composition of claim 7, wherein at least 80% of Formula (I) of the composition is Form D, at least 85% of Formula (I) of the composition is Form D, at least 90% of Formula (I) of the composition is Form D, at least 95% of Formula (I) of the composition is Form D, at least 98% of Formula (I) of the composition is Form D, or at least 99% of Formula (I) of the composition is Form D.

9-12. (canceled)

13. A composition comprising a crystalline form of Formula (I): 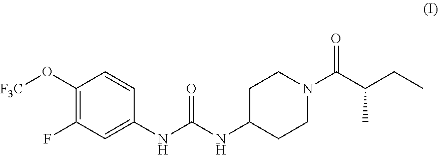 [SMILES: CC[C@H](C)C(=O)N1CCC(NC(=O)Nc2ccc(OC(F)(F)F)c(F)c2)CC1]
or a pharmaceutically acceptable salt thereof,
wherein the crystalline form of Formula (I) comprises Form C as characterized by an X-ray powder diffraction pattern substantially as set forth in Panel C of FIG. 1.

14. The composition of claim 13, wherein at least 80% of Formula (I) of the composition is Form C, at least 85% of Formula (I) of the composition is Form C, at least 90% of Formula (I) of the composition is Form C, at least 95% of Formula (I) of the composition is Form C, at least 98% of Formula (I) of the composition is Form C, or at least 99% of Formula (I) of the composition is Form C.

15-74. (canceled)

75. A method of generating a crystalline form of Formula (I) of claim 7, comprising:
(i) dissolving Formula (I) in a solvent system comprising a heptane and a solvent selected from the group consisting of dimethyl sulfoxide and n-methyl-2-pyrrolidone, and cooling the Formula (I) to a temperature of between −20° C. and 30° C. at a rate of between 0.1° C./hour and 600° C./hour;
(ii) dissolving Formula (I) to a concentration of at most 0.25 mg/ml in a solvent system comprising water and dimethyl formamide, and cooling to a temperature of between 0° C. and 30° C. at a rate of between 0.1° C./hour and 40° C./hour;
(iii) dissolving Formula (I) in a solvent system comprising a heptane and a solvent selected from the group consisting of ethanol, isopropyl alcohol, and ethyl acetate, and cooling to a temperature of between −20° C. and 30° C. at a rate of between 0.1° C./hour and 40° C./hour;
(iv) dissolving Formula (I) in a solvent system comprising a hexane and dimethyl sulfoxide, and cooling to a temperature of between −20° C. and 30° C. at a rate of between 0.1° C./hour and 40° C./hour;
(v) dissolving Formula (I) to a concentration of at least 0.4 mg/ml in a solvent system comprising a heptane and isopropyl alcohol, and cooling the solvent system to a temperature of between −20° C. and 30° C. at a rate of between 0.1° C./hour and 40° C./hour;
(vi) incubating Formula (I) in a heptane for at least 1 day; or
a combination thereof.

76-138. (canceled)

139. A method of treating a soluble epoxide hydrolase (sEH) mediated disorder or disease in a subject, comprising administering to the subject the composition of claim 13, thereby treating the disorder or disease in the subject.

140. The method of claim 139, wherein the sEH mediated disorder or disease is selected from the group consisting of pain, a seizure disorder, epilepsy, Parkinson's disease, Alzheimer's disease, depression, spinal cord injury, peripheral nerve injury, stroke, multiple sclerosis, cognitive disfunction, nephropathy, cardiomyopathy, wound healing and inflammation.

141. The method of claim 140, wherein the pain is neuropathic pain.

142. The method of claim 141, wherein the neuropathic pain is associated with a nerve injury.

143. The method of claim 142, wherein the nerve injury results from diabetes or other disease.

144. The method of claim 140, wherein the pain is diabetic neuropathic pain.

145. The method of claim 140, wherein the pain is inflammatory pain.

146. The method of claim 140, wherein the seizure disorder is epilepsy.

147-152. (canceled)

153. A composition comprising a crystalline form of Formula (I): 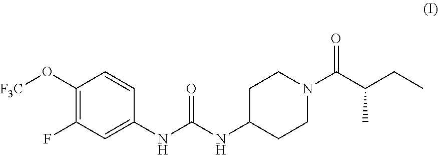 [SMILES: CC[C@H](C)C(=O)N1CCC(NC(=O)Nc2ccc(OC(F)(F)F)c(F)c2)CC1]
or a pharmaceutically acceptable salt thereof, wherein the crystalline form of Formula (I) comprises Form C as characterized by an X-ray powder diffraction pattern comprising peaks at 16.3±0.3°2θ, 16.1±0.3°2θ, and 3.2±0.3°2θ.

154. The composition of claim 153, wherein the X-ray powder diffraction pattern further comprises at least one, at least two, or at least three peaks selected from 21.6±0.3°2θ, 23.2±0.3°2θ, and 21.7±0.3°2θ.

155. The composition of claim 154, wherein the X-ray powder diffraction pattern further comprises at least one, at least two, or at least three peaks selected from 16.5±0.3°2θ, 21.4±0.3°2θ, and 10.7±0.3°2θ.

156. A composition comprising a crystalline form of Formula (I): 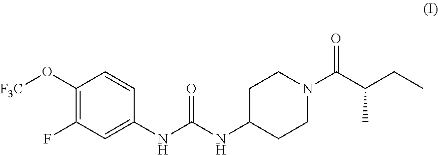 [SMILES: CC[C@H](C)C(=O)N1CCC(NC(=O)Nc2ccc(OC(F)(F)F)c(F)c2)CC1]
or a pharmaceutically acceptable salt thereof, wherein the crystalline form of Formula (I) comprises Form D as characterized by an X-ray powder diffraction pattern comprising peaks at 20.1±0.3°2θ, 18.3±0.3°2θ, and 18.1±0.3°2θ.

157. The composition of claim 156, wherein the X-ray powder diffraction pattern further comprises at least one or at least two peaks selected from 20.3±0.3°2θ and 17.1±0.3°2θ.

158. The composition of claim 157, wherein the X-ray powder diffraction pattern further comprises at least one, at least two, at least three, or at least four peaks selected from 3.4±0.3°2θ, 19.6±0.3°2θ, 23.4±0.3°2θ, and 25.1±0.3°2θ.

159-191. (canceled)

192. A method for generating a crystalline form of Formula (I), comprising:
(i) dissolving Formula (I) in a solvent system comprising water and methanol, and cooling the solvent system to a temperature of between 0° C. and 30° C. at a rate of between 60° C./hour and 600° C./hour;
(ii) incubating Formula (I) in water for at least one hour;
(iii) incubating Formula (I) in a polyethylene glycol (PEG) water mixture comprising at least 50% water by volume for at least one hour; or
(iv) a combination thereof.

193. The method of claim 192, wherein (i) comprises cooling the solvent system to a temperature of between 0° C. and 15° C. at a rate of between 60° C./hour and 150° C./hour.

194. The method of claim 192, wherein (ii) comprises incubating Formula (I) in water for at least one day.

195. The method of claim 192, wherein (iii) comprises incubating Formula (I) in a polyethylene glycol (PEG) water mixture comprising at least 50% water by volume for at least one day.

196. The method of claim 192, wherein (ii) and (iii) comprise incubating solid Formula (I).

197. The method of claim 192, wherein the crystalline form is at least 80% Form C, at least 85% Form C, at least 90% Form C, at least 95% Form C, at least 98% Form C, or at least 99% Form C.
